(19) United States
(12) Patent Application Publication
(10) Pub. No.: US 2005/0250716 A1
(43) Pub. Date: Nov. 10, 2005

(54) Immunostimulatory oligodeoxynucleotides

(75) Inventors: Walter Schmidt, Vienna (AT); Carola Schellack, Vienna (AT); Alena Egyed, Vienna (AT); Karen Lingnau, Vienna (AT)

Correspondence Address: FULBRIGHT & JAWORSKI L.L.P., 600 CONGRESS AVE., SUITE 2400, AUSTIN, TX, 78701, US

(73) Assignee: INTERCELL AG

(21) Appl. No.: 10/497,591

(22) PCT Filed: Dec. 5, 2002
(86) PCT No.: PCT/EP02/13791

§ 371 Date: Nov. 22, 2004

(30) Foreign Application Priority Data
Dec. 7, 2001 (AT) A 1924/2001

(51) Int. Cl. A61K 48/00

(52) U.S. Cl. 514/44

(57) ABSTRACT

The invention relates to the use of inununostimulatory deoxyinosine/deoxyuridine containing oligodeoxynucleotides for pharmaceutical application, such as treating and preventing chronic infectious diseases, acute decrements in air flow, parasitic infections and the like.

The present invention relates to new uses for oligodeoxynucleotides (ODNs) containing deoxyinosine and/or deoxyuridine residues.

ODNs containing deoxyinosine and/or deoxyuridine residues are disclosed in the Austrian patent applications A 1973/2000 and A 805/2001 (incorporated herein by reference).

Pharmaceutical uses of ODNS, especially palindromic ODNs or CpG containing ODNs are disclosed in EP .0 468 520 A2, WO96/02555, WO98/18810, WO98/37919, WO98/40100, WO99/51259 and WO99/56755, all incorporated herein by reference).

The object of the present invention is to provide further (medical) uses and methods for ODNs as defined above.

This object is solved by the use of an immunostimulatory oligodeoxynucleic acid molecule (ODN) having the structure according to the formula (I)

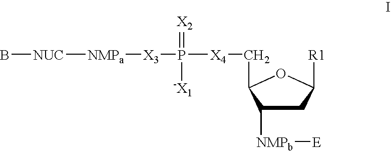

wherein

R1 is selected from hypoxanthine and uracile, any X is O or S,

any NMP is a 2′ deoxynucleoside monophosphate or monothiophosphate, selected from the group consisting of deoxyadenosine-, deoxyguanosine-, deoxyinosine-, deoxycytosine-, deoxyuridine-, deoxythymidine-, 2-methyl-deoxyinosine-, 5-methyl-deoxycytosine-, deoxypseudouridine-, deoxyribosepurine-, 2-amino-deoxyribosepurine-, 6-S-deoxyguanine-, 2-dimethyl-deoxyguanosine- or N-isopentenyl-deoxyadenosine-monophosphate or -monothiophosphat, NUC is a 2′ deoxynucleoside, selected from the group consisting of deoxyadenosine-, deoxyguanosine-, deoxyinosine-, deoxycytosine-, deoxyinosine-, deoxythymidine-, 2-methyl-deoxyuridine-, 5-methyl-deoxycytosine-, deoxypseudouridine-, deoxyribosepurine-, 2-amino-deoxyribosepurine-, 6-S-deoxyguanine-, 2-dimethyl-deoxyguanosine- or N-isopentenyl-deoxyadenosine,

a and b are integers from 0 to 100 with the proviso that a+b is between 4 and 150,

B and E are common groups for 5′ or 3′ ends of nucleic acid molecules

for the preparation of a pharmaceutical preparation, preferably with the proviso that said preparation is not a vaccine.

Such ODNs and their use in vaccination have been described in the Austrian patent applications A 1973/2000 and A 805/2001. It has now surprisingly turned out that these dI and/or dU containing ODNs may be used in all instances wherein palindromic ODNs or CpG containing ODNs (palindromic or not) have been used or proposed. The ODNs to be used in the present invention often show less side effects and improved properties over the “classical” ODNs (comprising only A, T, C and G).

For example, ODNs according to the present invention do not induce the systemic production of pro-inflammatory cytokines, such as TNF-α and IL-6, thus reducing the induction of potential harmful side reactions.

Whereas certain immunostimulatory effects had been described for inosine containing RNA molecules, such as poly-IC or the molecules mentioned in WO98/16247, it surprisingly turned out that short deoxynucleic acid molecules containing deoxyuridine and/or deoxyinosine residues, may be good immunostimulating ODNs.

In addition, the dU/dI containing ODNs according to the present invention are—in contrast to ODNs based on the specific CpG motif—not dependent on a specific motif or a palindromic sequence as described for the CpG oligonucleotides.

Therefore, one group of dU/dI-ODNs according to the present invention may preferably contain a C(dI/dU) motif (and therefore ODNs described in these incorporated references, wherein one or more guanosine residues are replaced with deoxy(uridine/inosine) residues are preferred embodiments of the present ODNs). However, such a motif is not necessary for its principle immunostimulatory property, since dU/dI-ODNs with an (uridine/inosine) not placed in a C(dI/dU) or (dI/dU)C context exhibit immunostimulatory properties as well.

The dU/dI-ODN according to the present invention is therefore a DNA molecule containing a deoxy(uridine/inosine) residue which is preferably provided in single stranded form.

The dU/dI-ODN according to the present invention may be isolated through recombinant methods or chemically synthesized. In the latter case, the dU/dI-ODN according to the present invention may also contain modified oligonucleotides which may be synthesized using standard chemical transformations, such as methylphosphonates or other phosphorous based modified oligonucleotides, such as phosphotriesters, phosphoamidates and phosphorodithiorates. Other non-phosphorous based modified oligonucleotides can also be used, however, monophosphates or monothiophosphates being the preferred 2′deoxynucleoside monophosphate to be used in the present invention.

The NMPs of the dU/dI-ODNs according to the present invention are preferably selected from the group consisting of deoxyadenosine-, deoxyguanosine-, deoxyinosine-, deoxycytosine-, deoxyinosine-, deoxythymidine-, 2-methyl-deoxyuridine-, 5-methyl-deoxycytosine-monophosphate or -monothiophosphate (as usual, the phosphate or thiophosphate group is 5′ of the deoxyribose). Whereas it is essential for the ODNs based on the CpG motif that this motif is unmethylated, this is surprisingly not the case for the ODNs according to the present invention, wherein e.g. 2-methyl-deoxyinosine or 5-methyl-deoxycytosine residues have no general negative effect on immunostimulatory properties of the ODNs according to the present invention. Alternatively, instead of the 2-deoxy- forms of the NMPs, also other, especially inert, groups may be present at the 2-site of the ribose group, such as e.g. —F, —NH₂, —CH₃, especially —CH₃. Of course, —OH and SH groups are excluded for the dU/dI-ODNs according to the present invention to be present on the 2′-site of the ribose, especially the ribose residue for the (uridine/inosine) NMP.

The length of the ODNs according to the present invention is in the range of the standard ODNs used according to the prior art. Therefore molecules with a total length under 4 and above 150 show gradually decreasing immunostimulatory potential. Preferred ODNs contain between 10 and 60, especially between 15 and 40 bases (nucleosides), implying that a+b in formula I is between 10 and 60, preferably between 15 and 40 in these preferred embodiments.

Whereas the ribonucleic acid molecules containing inosine and cytidine described to be immunostimulatory in the prior art have been large and relatively undefined polynucleic acids with molecular weights far above 200,000 (a commercially available polyinosinic-polycytidylic acid from Sigma Chemicals has a molecular weight ranging from 220,000 to 460,000 (at least 500-1000 C+I residues). The molecules according to the present invention are DNA molecules of much shorter length with a well defined length and composition, being highly reproducible in products.

It is further preferred that the deoxy(uridine/inosine) containing NMP of the dU/dI-ODNs according to formula I is a monothiophosphate with one to four sulfur atoms and that also further NMPs, especially all further NMPs, are present as nucleoside monothiophosphates, because such ODNs display higher nuclease resistance (it is clear for the present invention that the “mono” in the “monothiophosphates” relates to the phosphate, i.e. that one phosphate group (one phosphor atom) is present in each NMP). Preferably, at least one of X₁ and X₂ is S and at least one of X₃ and X₄ is O in the NMPs according to the present invention. Preferably, X₃ and X₄ are O. (X₃ may be (due to synthesis of the NMP) derived e.g. from the phosphate group or from the 3′-group of the NMP-ribose).

Preferably the ODNs according to the present invention contain the sequence

| | | | | | | | |
|---|---|---|---|---|---|---|---|
| tcc | atg | acu | ttc | ctg | ctg | atg | ct |
| nhh | hhh | wdu | dhh | hhh | hhh | wn | |
| hhh | wdu | dhh | h | | | | |

wherein

any n is a 2′-deoxynucleoside monophosphate or monothiophosphate, selected from the group consisting of deoxyadenosine-, deoxyguanosine-, deoxycytosine- or deoxythymidine-monophosphate or -monothiophosphate,

any h is a 2′-deoxynucleoside monophosphate or monothiophosphate, selected from the group consisting of deoxyadenosine-, deoxycytosine- or deoxythymidine-monophosphate or -monothiophosphate

u is deoxyuridine-monophosphate or -monothiophosphate, any w is a 2′-deoxynucleoside monophosphate or monothiophosphate, selected from the group consisting of deoxyadenosine- or deoxythymidine-monophosphate or -monothiophosphate, and any d is a 2′-deoxynucleoside monophosphate or monothiophosphate, selected from the group consisting of deoxyadenosine-, deoxyguanosine- or deoxythymidine-monophosphate or -monothiophosphate.

Further preferred ODNs according to the present invention contain the sequence

wdu, wdud, wdudn
or
wdudud,

wherein w, d, u and n are defined as above.

Preferably the ODNs according to the present invention contain the sequence

| | | | | | | |
|---|---|---|---|---|---|---|
| hhh | wdi | dhh | h | | | |
| nhh | hhh | wdi | nhh | hhh | hhh | wn, |
| nhh | wdi | din | hhh | hdi | ndi | nh, |
| nhh | hhh | wdi | dhh | hhh | hhh | wn or |
| nhh | wdi | did | hhh | hdi | ddi | dh, |

wherein

any n is a 2′-deoxynucleoside monophosphate or monothiophosphate, selected from the group consisting of deoxyadenosine-, deoxyguanosine-, deoxycytosine- or deoxythymidine-monophosphate or -monothiophosphate,

any h is a 2′-deoxynucleoside monophosphate or monothiophosphate, selected from the group consisting of deoxyadenosine-, deoxycytosine- or deoxythymidine-monophosphate or -monothiophosphate

i is deoxyinosine-monophosphate or -monothiophosphate, any w is a 2′-deoxynucleoside monophosphate or monothiophosphate, selected from the group consisting of deoxyadenosine- or deoxythymidine-monophosphate or -monothiophosphate, and any d is a 2′-deoxynucleoside monophosphate or monothiophosphate, selected from the group consisting of deoxyadenosine-, deoxyguanosine- or deoxythymidine-monophosphate or -monothiophosphate.

Preferred ODNs according to the present invention contain one or more of the sequence

gacitt,
iacitt,
gaictt,
iaictt,

wherein

a is deoxyadenosine-monophosphate or -monothiophosphate,

g is deoxyguanosine-monophosphate or -monothiophosphate,

i is deoxyinosine-monophosphate or -monothiophosphate,

c is deoxycytosine-monophosphate or -monothiophosphate and

t is deoxythymidine-monophosphate or -monothiophosphate.

As outlined above, a specific motif (such as CpG or a palindrome) is not necessary for the dU/dI-ODNs according to the present invention.

However, ODNs containing a C(dI/dU) motif are preferred so that in a preferred embodiment the ODN according to formula I contains at least one 2′deoxycytosine-monophosphate or -monothiophosphate 3′-adjacent to a 2′-deoxyuridine-monophosphate or -monothiophosphate and/or

at least one 2′deoxycytosine-monophosphate or -monothiophosphate 3′-adjacent to a 2′-deoxyinosine-monophosphate or -monothiophosphate.

Preferred ODNs according to the present invention contain one or more of the sequence

gacutt,
uacutt,
gauctt,
uauctt,

wherein

a is deoxyadenosine-monophosphate or -monothiophosphate,

g is deoxyguanosine-monophosphate or -monothiophosphate,

u is deoxyuridine-monophosphate or -monothiophosphate,

c is deoxycytosine-monophosphate or -monothiophosphate and

t is deoxythymidine-monophosphate or -monothiophosphate.

The dU/dI-ODNs according to the present invention are especially suitable for application in the pharmaceutical field, e.g, to be applied as a medicine to an animal or to humans.

The present ODNs have immunopharmacological activity and are efficacious against malignant tumors as reported for synthetic RNAs. Unlike the synthetic RNAs, these synthetic DNAs may presumably be useful remedies because of their minimized side effects, such as fever, as well as solving the following problems associated with synthetic RNA.

(1) The molecular weight must be high (e.g. 30000 Da or more ) to ensure a satisfactory pharmacological activity, and this requires enzymatic synthesis. The products thus obtained, when used as a drug, can contain enzymes left unremoved and are very unsatisfactory in terms of safety.

(2) It is difficult by enzymatic synthesis to accurately control the molecular-weight distribution of the products, and hence the molecular-weight distribution is generally different among production lots. This is unfavorable in terms of specification setting for drugs.

Double-stranded, linear DNA is a double helical complex composed of a single-stranded, linear DNA as described above [DNA(A)] and a second single-stranded, linear DNA [DNA(B)] with base sequence which are partially or completely complementary with those of DNA(A). Either DNA(A), DNA(B) or the both must contain at least one sequence represented by the general formula (I). Such double-stranded, linear DNAs alone have the same immunostimulatory activity as single-stranded, linear DNAs do.

Mixtures of a single-stranded linear DNA and a double-stranded, linear DNA are also included in this invention.

These DNAs may also be used in the form of medicinally approved salts. For example, sodium salts can be obtained by adding sodium hydroxide to an aqueous solution of DNA of this invention to adjust the pH to 7, followed by lyophilization. These DNAs may also be used as a complex with a polycationic compound, such as poly-L-lysine (hereinafter abbreviated as PLL). Such complex can be prepared, for example, by mixing an aqueous solution of DNA of this invention with an aqueous solution of PLL so that the DNA-PLL weight ratio will be about 4:3.

The pharmaceutical preparations of this invention may be used alone or in combination with other therapeutic means against such diseases the outbreak of which can be suppressed, or the progress of which can be arrested or delayed, by the functions of the immune system. As examples of such diseases, may be mentioned, among others, malignant tumors, autoimmune diseases, immunodeficiency diseases and infectious diseases. Malignant tumors are diseases such as gastric cancer, colorectal cancer, breast cancer, skin cancer, liver cancer, uterine cancer, reticulosarcomas, lymphosarcomas, leukemias, lymphomas and like diseases. Autoimmune diseases are the diseases which are considered to result from impaired self-recognizing function of the immune system, such as rheumatoid arthritis, SLE, juvenile onset diabetes, multiple sclerosis, autoimmune hemolytic anemia and myasthenia gravis, which are considered to be effectively cured by drugs having immunopharmacological activity. Infectious diseases are the diseases caused by infection with bateria, viruses or protozoans, and are considered to be effectively cured by drugs having immunopharmacological activity (such as interferon). As described later, DNAs of this invention are capable of effectively cure infectious diseases, especially viral diseases. Immunodeficiency diseases are the diseases in which the functions of immune system are suppressed or lost, such as agammaglobulinemia and acquired immunodeficiency syndromes. Among the patients of these diseases, the morbidity of infectious diseases and malignant tumors is high, thus adversely affecting recuperation. DNAs of this invention, which are efficacious against malignant tumors and are also capable of inducing interferon, are expected to encourage the recuperation of the patients suffering immunodeficiency diseases by curing the malignant tumors and infectious diseases which are likely to concur in these patients.

Single- and double-stranded, linear DNAs of this invention may be administerd to animal and human bodies subcutaneously, intravenously, intramuscularly, intratumorally, orally or into the rectum, and the suitable administration route should be selected case by case depending on the type of disease and the conditions of the patient. For example, intratumoral or subcutaneous administration is preferable in the case of malignant tumors. The proper dose to humans is e.g. 1 to 1000 mg/day when administered into the rectum or orally, and 0.01 to 100 mg/day when administered subcutaneously, intravenously, intratumorally or intramuscularly. Administration should be repeated once or twice per one to seven days, preferably once per one or two days, and the frequency of administration may be varied and the period of administration may be further prolonged, as required.

When administering single- or double-stranded, linear DNAs of this invention to animal and human bodies subcutaneously, intravenously, intramuscularly or intratumorally, it is preferable to appply it in the form of an injection prepared by dissolving the DNA in an aqueous solution which is nearly neutral (pH 5 to 8) with a physiological osmotic pressure. As examples of such an aqueous solution, may be mentioned the isotonic sodium chloride solution specified in Pharmacopoeia of Japan, and aqueous solutions containing salts, compounds, additives or diluents medicinally approved. The single- and double-stranded, linear DNAs of this invention may be used as an injection either in the form of an aqueous solution as described above or in the form of solid obtained by lyophylizing the same.

The single- and double-stranded, linear DNAs of this invention, when orally administered to animal and human bodies, may be used in the form of capsules, granules, pills, fine granules, tablets or syrup, as in the case of common drugs.

According to one further aspect of the present invention lymphocytes can either be obtained from a subject and stimulated ex vivo upon contact with an appropriate oligonucleotide; or a (non-methylated) dI/dU containing oligonucleotide can be administered to a subject to facilitate in vivo activation of a subject's lymphocytes. Activated lymphocytes, stimulated by the methods described herein (e.g. either ex vivo or in vivo), can boost a subject's immune response. The immunostimulatory oligonucleotides can therefore be used to treat, prevent or ameliorate an immune system deficiency (e.g., a tumor or cancer or a viral, fungal, bacterial or parasitic infection in a subject. In addition, immunostimulatory oligonucleotides can also be administered as a vaccine adjuvant, to stimulate a subject's response to a vaccine. Further, the ability of immunostimulatory cells to induce leukemic cells to enter the cell cycle, suggests a utility for treating leukemia by increasing the sensitivity of chronic leukemia cells and then administering conventional ablative chemotherapy.

Moreover, in vivo administration of ODNs according to the present invention should prove useful for treating diseases such as systemic lupus erythematosus, sepsis and autoimmune diseases. In addition, methylation dI/dU containing antisense oligonucleotides or oligonucleotide probes would not initiate an immune reaction when administered to a subject in vivo and therefore would be safer than corresponding unmethylated oligonucleotides.

Preferably, the ODN according to the present invention is an oligonucleotide that is relatively resistant to in vivo degradation (e.g. via an exo- or endo-nuclease). Preferred stabilized oligonucleotides of the instant invention have a modified phosphate backbone. Especially preferred oligonucleotides have a phosphorothioate modified phosphate backbone (i.e. at least one of the phosphate oxygens is replaced by sulfur). Other stabilized oligonucleotides include: nonionic DNA analogs, such as alkyl- and aryl- phosphonates (in which the charged phosphonate oxygen is replaced by an alkyl or aryl group), phosphodiester and alkylphosphotriesters, in which the charged oxygen moiety is alkylated. Oligonucleotides which contain a diol, such as tetraethyleneglycol or hexaethyleneglycol, at either or both termini have also been shown to be substantially resistant to nuclease degradation.

Examples of oligonucleotide delivery complexes include oligonucleotides associated with: a sterol (e.g. cholesterol), a lipid (e.g. a cationic lipid, virosome or liposome), or a target cell specific binding agent (e.g. a ligand recognized by target cell specific receptor). Preferred complexes must be sufficiently stable in vivo to prevent significant uncoupling prior to internalization by the target cell. However, the complex should be cleavable under appropriate conditions within the cell so that the oligonucleotide is released in a functional form.

An “immune system deficiency” (for which the present ODNs may be applied) shall mean a disease or disorder in which the subject's immune system is not functioning in normal capacity or in which it would be useful to boost a subject's immune response for example to eliminate a tumor or cancer (e.g. tumors of the brain, lung (e.g. small cell and non-small cell), ovary, breast, prostate, colon, as well as other carcinomas and sarcomas) or a viral (e.g. HIV, herpes), fungal (e.g. Candida sp.), bacterial or parasitic (e.g. Leishmania, Toxoplasma) infection in a subject.

A “disease associated with immune system activation” shall mean a disease or condition caused or exacerbated by activation of the subject's immune system. Examples include systemic lupus erythematosus, sepsis and autoimmune diseases such as rheumatoid arthritis and multiple sclerosis.

A “subject” shall mean a-human or vertebrate animal including a dog, cat, horse, cow, pig, sheep, goat, chicken, monkey, rat, mouse, etc.

The ODNs according to the present invention mediate B cell activation and IgM secretion. Similar stimulation may be seen using B cells from C3H/HeJ mice, eliminating the possibility that lipopolysaccharide (LPS) contamination could account for the results.

A CpdI/dU motif may be an important element present in ODNs that activate B cells.

The bases flanking a given CpdI/dU dinucleotide may play an important role in determining the B cell activation induced by an ODN. The optimal stimulatory motif was determined to consist of a CpdI/dU flanked by two 5′ purines (preferably a GpA dinucleotide) and two 3′ pyrimidines (preferably a TpT or TpC dinucleotide). Mutations of ODN to bring the CpdI/dU motif closer to this ideal improved stimulation while mutations that disturbed the motif reduced stimulation. On the other hand, mutations outside the CpdI/dU motif did not reduce stimulation.

ODNs shorter than 8 bases may be non-stimulatory. ODNs containing Gs at both ends may show increased stimulation, particularly if the the ODN are rendered nuclease resistant by phosphorothioate modification of the terminal internucleotide linkages.

Other octamer ODNs containing a 6 base palindrome with a TpC dinucleotide at the 5′ end may also be active if they were close to the optimal motif.

A marked induction of NK activity among spleen cells cultured with CpdI/dU ODN may be observed. In contrast, there may be relatively no induction in effectors that had been treated with non-CpdI/dU control ODN.

Teleologically, it appears likely that lymphocyte activation by the CpdI/dU motif represents an immune defense mechanism that can thereby distinguish bacterial from host DNA. Host DNA would induce little or no lymphocyte activation due to it CpdI/dU suppression and methylation. Bacterial DNA would cause selective lymphocyte activation in infected tissues. Since the CpdI/dU pathway synergizes with B cell activation through the antigen receptor, B cells bearing antigen receptor specific for bacterial antigens would receive. one activation signal through cell membrane Ig and a second signal from bacterial DNA, and would therefore tend to be preferentially activated. The interrelationship of this pathway with other pathways of B cell activation provide a physiologic mechanism employing a polyclonal antigen to induce-specific responses.

For use in the instant invention, oligonucleotides can be synthesized de novo using any of a number of procedures well known in the art. For example, the ss-cyanoethyl phosphoramidite method. These chemistries can be performed by a variety of automated oligonucleotide synthesizers available in the market. Alternatively, oligonucleotides can be prepared from existing nucleic acid sequences (e.g. genomic or cDNA) using known techniques, such as those employing restriction enzymes, exonucleases or endonucleases.

For use in vivo, oligonucleotides are preferably relatively resistant to degradation (e.g. via endo- and exo- nucleases). Oligonucleotide stabilization can be accomplished via phosphate backbone modifications. A preferred stabilized oligonucleotide has a phosphorothioate modified backbone. The pharmacokinetics of phosphorothioate ODN show that they have a systemic half-life of forty-eight hours in rodents and suggest that they may be useful for in vivo applications. Phosphorothioates may be synthesized using automated techniques employing either phosphoramidate or H phosphonate chemistries. Aryl- and alkyl- phosphonates can be made; and alkylphosphotriesters (in which the charged oxygen moiety is alkylated) can be prepared by automated solid phase synthesis using commercially available reagents. Methods for making other DNA backbone modifications and substitutions have been described.

For administration in vivo, oligonucleotides may be associated with a molecule that results in higher affinity binding to target cell (e.g. B-cell and natural killer (NK) cell) surfaces and/or increased cellular uptake by target cells to form an “oligonucleotide delivery complex”. Oligonucleotides can be tonically, or covalently associated with appropriate molecules using techniques which are well known in the art. A variety of coupling or crosslinking agents can be used e.g. protein A, carbodiimide, and N succinimidyl-3-(2-pyridyldithio) propionate (SPDP). Oligonucleotides can alternatively be encapsulated in liposomes or virosomes using well-known techniques.

Based on their immunostimulatory properties, dI/dU-containing ODNs, especially oligonucleotides containing at least one unmethylated CpdI/dU dinucleotide, can be administered to a subject in vivo to treat an immune system deficiency”. Alternatively, such oligonucleotides can be contacted with lymphocytes (e.g. B cells or NK cells) obtained from a subject having an immune system deficiency ex vivo and activated lymphocytes can then be reimplanted in the subject.

Immunostimulatory oligonucleotides can also be administered to a subject in conjunction with a vaccine, as an adjuvant, to boost a subject's immune system to effect better response from the vaccine. Preferably the dI/dU ODN is administered slightly before or at the same time as the vaccine.

Preceding chemotherapy with an immunostimulatory oligonucleotide should prove useful for increasing the responsiveness of the malignant cells to subsequent chemotherapy. DI/dU-ODNs, especially CpdI/dU ODNs, also increased natural killer cell activity in both human and murine cells. Induction of NK activity may likewise be beneficial in cancer immunotherapy.

ODNs according to the present invention (containing dI and/or dU residues) that are complementary to certain target sequences can be synthesized and administered to a subject in vivo. For example, antisense oligonucleotides hybridize to complementary mRNA, thereby preventing expression of a specific target gene.

The sequence-specific effects of antisense oligonucleotides have made them useful research tools for the investigation of protein function.

In addition, oligonucleotide probes (i.e. oligonucleotides with a detectable label) can be administered to a subject to detect the presence of a complementary sequence based on detection of bound label. In vivo administration and detection of oligonucleotide probes may be useful for diagnosing certain diseases that are caused or exacerbated by certain DNA sequences (e.g. systemic lupus erythematosus, sepsis and autoimmune diseases).

Antisense oligonucleotides or oligonucleotide probes in which any or all dI/dU dinucleotide is methylated, would not produce an immune reaction when administered to a subject in vivo and therefore would be safer than the corresponding non-methylated dI/dU containing oligonucleotide.

For use in therapy, an effective amount of an appropriate oligonucleotide alone or formulated as an oligonucleotide delivery complex can be administered to a subject by any mode allowing the oligonucleotide to be taken up by the appropriate target cells (e.g. B-cells and NK cells). Preferred routes of administration include oral and transdermal (e.g. via a patch). Examples of other routes of administration include injection (subcutaneous, intravenous, parenteral, intraperitoneal, intrathecal, etc.). The injection can be in a bolus or a continuous infusion.

An oligonucleotide alone or as an oligonucleotide delivery complex can be administered in conjunction with a pharmaceutically acceptable carrier. As used herein, the phrase “pharmaceutically acceptable carrier” is intended to include substances that can be coadministered with an oligonucleotide or an oligonucleotide delivery complex and allows the oligonucleotide to perform its intended function. Examples of such carriers include solutions, solvents, dispersion media, delay agents, emulsions and the like. The use of such media for pharmaceutically active substances are well known in the art. Any other conventional carrier suitable for use with the oligonucleotides falls within the scope of the instant invention.

The language “effective amount” of an oligonucleotide refers to that amount necessary or sufficient to realize a desired biologic effect. For example, an effective amount of a dI/dU-ODN, especially an oligonucleotide containing at least one methylated CpdI/dU, for treating an immune system deficiency could be that amount necessary to eliminate a tumor, cancer, or bacterial, viral or fungal infection. An effective amount for use as a vaccine adjuvant could be that amount useful for boosting a subject's immune response to a vaccine. An “effective amount” of an oligonucleotide lacking a non-methylated dI/dU for use in treating a disease associated with immune system activation, could be that amount necessary to outcompete non-methylated dI/du containing nucleotide sequences. The effective amount for any particular application can vary depending on such factors as the disease or condition being treated, the particular oligonucleotide being administered, the size of the subject, or the severity of the disease or condition. One of ordinary skill in the art can empirically determine the effective amount of a particular oligonucleotide without necessitating undue experimentation.

ODNs according to the present invention, especially unmethylated CpdI/dU containing oligonucleotides, are directly mitogenic for lymphocytes (e.g. B cells and NK cells). However, it is likely that B cell activation would not be totally nonspecific. B cells bearing antigen receptors specific for bacterial products could receive one activation signal through cell membrane Ig, and a second from bacterial DNA, thereby more vigorously triggering antigen specific immune responses.

As with other immune defense mechanisms, the response to bacterial DNA could have undesirable consequences in some settings. For example, autoimmune responses to self antigens would also tend to be preferentially triggered by bacterial infections, since autoantigens could also provide a second activation signal to autoreactive B cells triggered by bacterial DNA. Indeed the induction of autoimmunity by bacterial infections is a common clinical observance. For example, the autoimmune disease systemic lupus erythematosus, which is: i) characterized by the production of anti-DNA antibodies; ii) induced by drugs which inhibit DNA methyltransferase; and iii) associated with reduced DNA methylation, is likely triggered at least in part by activation of DNA-specific B cells.

Further, sepsis, which is characterized by high morbidity and mortality due to massive and nonspecific activation of the immune system may be initiated by bacterial DNA and other products released from dying bacteria that reach concentrations sufficient to directly activate many lymphocytes.

Lupus, sepsis and other “diseases associated with immune system activation” may be treated, prevented or ameliorated by administering to a subject ODNs according to the present invention, especially oligonucleotides lacking an unmethylated CpdI/dU dinucleotide (e.g. oligonucleotides that do not include a CpdI/dU motif or oligonucleotides in which the CpdI/dU motif is methylated) to block the binding of unmethylated CpdI/dU containing nucleic acid sequences. oligonucleotides lacking an unmethylated CpdI/dU motif can be administered alone or in conjunction with compositions that block an immune cell's reponse to other mitogenic bacterial products (e.g. LPS).

Lupus is commonly thought to be triggered by bacterial or viral infections. Such infections have been reported to stimulate the production of nonpathogenic antibodies to single stranded DNA. These antibodies likely recognize primarily bacterial sequences. As disease develops in lupus, the anti-DNA antibodies shift to pathogenic antibodies that are specific for double-stranded DNA. These antibodies would have increased binding for nucleic acid sequences and their production would result from a breakdown of tolerance in lupus. Alternatively, lupus may result when a patient's DNA becomes hypomethylated, thus allowing anti-DNA antibodies specific for unmethylated ODNs to bind to self DNA and trigger more widespread autoimmunity through the process referred to as “epitope spreading”.

In either case, it may be possible to restore tolerance in lupus patients by coupling antigenic oligonucleotides to a protein carrier such as gamma globulin (IgG). Calfthymus DNA complexed to gamma globulin has been reported to reduce anti-DNA antibody formation.

Further, the ability of the nucleic acid sequences of the invention described herein to induce leukemic cells to enter the cell cycle supports their use in treating leukemia by increasing the sensitivity of chronic leukemia cells followed by conventional ablative chemotherapy, or by combining the nucleic acid sequences with other immunotherapies.

The nucleic acid sequences of the invention are also useful for stimulating natural killer cell (NK) lytic acitivity in a subject such as a human. The nucleic acid sequences of the invention are also useful for stimulating B cell proliferation in a subject such as a human. In another aspect, the nucleic acid sequences of the invention are useful as an adjuvant for use during antibody production in a mammal. Furthermore, the present nucleic acid sequences can be administered to treat or prevent the symptoms of an asthmatic disorder by redirecting a subject's immune response from Th2 to Th1.

The present invention is further based on the finding that nucleic acids containing at least one dI/dU residue, especially containing unmethylated cytosine-deoxyinosine/deoxyuridine (CpdI/dU) dinucleotides, affect the immune response in a subject by activating natural killer cells (NK) or redirecting a subject's immune response from a Th2 to a Th1 response by inducing monocytic and other cells to produce Th1 cytokines. These ODNs according to the present invention, especially the nucleic acids containing at least one unmethylated CpdI/dU can be used to treat pulmonary disorders having an immunologic component, such as asthma or environmentally induced airway disease. Therefore also a method of treating a subject having or at risk of having an acute decrement in air flow is provided comprising administering a therapeutically effective amount of nucleic acids containing at least one ODN according to the present invention, especially unmethylated CpdI/dU.

In another embodiment, a method of treating a subject having or at risk of-having an inflammatory response to lipopolysaccharide by administering a therapeutically effective amount of an ODN according to the present invention, especially nucleic acids containing at least one unmethylated CpdI/dU, is also provided. The invention also provides a method of modifying the level of a cytokine in a subject having or at risk of having inhaled lipopolysaccharide by administering a therapeutically effective dI/dU containing ODN, especially nucleic acid containing at least one unmethylated CpdI/dU.

The term “acute” refers to a condition having a short and relatively severe course. A “decrement in air flow” is a decreas terms “lung function” and “pulmonary function” are used interchangeably and shall be interpreted to mean physically measurable operations of a lung including but not limited to inspiratory flow rate, expiratory flow rate, and lung volume. Methods of quantitatively determining pulmonary function.are used to measure lung function.

Methods of measuring pulmonary function most commonly employed in clinical practice involve timed measurement of inspiratory and expiratory maneuvers to measure specific parameters. For example, forced vital capacity (FVC) measures the total volume in liters exhaled by a patient forcefully from a deep initial inspiration. This parameter, when evaluated in conjunction with the forced expired volume in one second (FEV,), allows bronchoconstriction to be quantitatively evaluated. A problem with forced vital capacity determination is that the forced vital capacity maneuver (i.e., forced exhalation from maximum inspiration to maximum expiration) is largely technique dependent. In other words, a given patient may produce different FVC values during a sequence of consecutive FVC maneuvers. The FEF 25-75 or forced expiratory flow determined over the midportion of a forced exhalation maneuver tends to be less technique dependent than the FVC. Similarly, the FEV1 tends to be less technique dependent than FVC. In addition to measuring volumes of exhaled air as indices of pulmonary function, the flow in liters per minute measured over differing portions of the expiratory cycle can be useful in determining the status of a patient's pulmonary function. In particular, the peak expiratory flow, taken as the highest air flow rate in liters per minute during a forced maximal exhalation, is well correlated with overall pulmonary function in a patient with asthma and other respiratory diseases.

By “therapeutically effective amount” is meant the quantity of a compound according to the invention necessary to prevent, to cure or at least partially arrest symptoms in a subject. A subject is any mammal, preferably a human. Amounts effective for therapeutic use will, of course, depend on the severity of the disease and the weight and general state of the subject. Typically, dosages used in vitro may provide useful guidance in the amounts useful for in situ administration of the pharmaceutical composition, and animal models may be used to determine effective dosages for treatment of particular disorders.

In another embodiment, the invention further provides a method of treating a subject having or at risk of having an inflammatory response to LPS by administering to the subject a therapeutically effective amount of a dI/dU containing ODN, especially a nucleic acid sequence containing at least one unmethylated CpdI/dU.

Examples of diseases which can be associated with Gram-negative bacterial infections or endotoxemia include bacterial meningitis, neonatal sepsis, cystic fibrosis, inflammatory bowel disease and liver cirrhosis, Gram-negative pneumonia, Gram-negative abdominal abscess, hemorrhagic shock and disseminated intravascular coagulation. Subjects who are leukopenic or neutropenic, including subjects treated with chemotherapy or immunocompromised subjects (for example with AIDS), are particularly susceptible to bacterial infection and the subsequent effects of endotoxin.

By “lipopolysaccharide” or “LPS” is meant a compound composed of a heteropolysaccharide (which contains somatic 0 antigen) covalently bound to a phospholipid moiety (lipid a). LPS is a major component of the cell wall. of Gram-negative bacteria. By “endotoxin” is meant a heat-stable toxin associated with the outer membranes of certain Gram-negative bacteria, including the enterobacteria, brucellae, neisseriae, and vibrios. Endotoxin; normally released upon disruption of the bacterial cells, is composed of lipopolysaccharide molecules (LPS) and any associated proteins. The phospholipid moiety of LPS, lipid a, is associated with LPS toxicity.

When injected in large quantities endotoxin produces hemorrhagic shock and severe diarrhea; smaller amounts cause fever, altered resistance to bacterial infection, leukopenia followed by leukocytosis, and numerous other biologic effects. Endotoxin is a type of “bacterial pyrogen,” which is any fever-raising bacterial product. The terms “endotoxin,” “LPS,” and “lipopolysaccharide” as used herein are essentially synonymous.

The invention further provides a method of treating a subject having or at risk of having an inflammatory response to LPS. It is known that LPS produces an inflammatory response in normal and asthmatic patients. By “inflammatory response” is meant an accumulation of white blood cells, either systemically or locally at the site of inflammation. The inflammatory response may be measured by many methods well known in the art, such as the number of white blood cells (WBC), the number of polymorphonuclear neutophils (PMN), a measure of the degree of PMN activation, such as luminal enhanced-chemiluminescence, or a measure of the amount of cytokines present. The term “cytokine” is used as a generic name for a diverse group of soluble proteins and peptides which act as humoral regulators at nano- to picomolar concentrations and which, either under normal or pathological conditions, modulate the functional activities of individual cells and tissues. These proteins also mediate interactions between cells directly and regulate processes taking place in the extracellular environment.

The invention may be used to treat individuals who are “at risk” of developing a acute decrement in airflow or who are at risk of LPS exposure. These individuals may be identified by any diagnostic means, or by epidemiological evidence such as exposure data. These individuals may be treated by a method of the invention prior to, at the time of, or after the actual onset of the clinical appearance. The “clinical appearance” can be any sign or symptom of the disorder.

This invention further provides administering to a subject having or at risk of having an inflammatory response to inhaled LPS, a therapeutically effective dose of a pharmaceutical composition containing the compounds of the present invention and a pharmaceutically acceptable carrier. “Administering” the pharmaceutical composition of the present invention may be accomplished by any means known to the skilled artisan.

The pharmaceutical compositions according to the invention are in general administered topically, intravenously, orally, parenterally or as implants, and even rectal use is possible in principle. Suitable solid or liquid pharmaceutical preparation forms are, for example, granules, powders, tablets, coated tablets, (micro)capsules, suppositories, syrups, emulsions, suspensions, creams, aerosols, drops or injectable solution in ampule form and also preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems.

The pharmaceutical compositions are preferably prepared and administered in dose units. Solid dose units are tablets, capsules and suppositories. For treatment of a patient, depending on activity of the compound, manner of administration, nature and severity of the disorder, age and body weight of the patient, different daily doses are necessary. Under certain circumstances, however, higher or lower daily doses may be appropriate. The administration of the daily dose can be carried out both by single administration in the form of an individual dose unit or else several smaller dose units and also by multiple administration of subdivided doses at specific intervals. The pharmaceutical compositions according to the invention may be administered locally or systemically. By “therapeutically effective dose” is meant the quantity of a compound according to the invention necessary to prevent, to cure or at least partially arrest the symptoms of the disorder and its complications. Amounts effective for this use will, of course, depend on the severity of the disease and the weight and general state of the patient. Typically, dosages used in vitro may provide useful guidance in the amounts useful for in situ administration of the pharmaceutical composition, and animal models may be used to determine effective dosages for treatment of particular disorders.

The following examples are intended to illustrate but not to limit the invention in any manner, shape, or form, either explicitly or implicitly. While they are typical of those that might be used, other procedures, methodologies, or techniques known to those skilled in the art may alternatively be used.

These nucleic acids can be used as an adjuvant, specifically to induce an immune response against an antigenic protein.

In one embodiment, the invention provides a method of inducing an immune response in a subject by administering to the subject a therapeutically effective amount of such a nucleic acid encoding an antigenic protein and a therapeutically effective amount of an oligonucleotide containing at least one ODN according to the present invention.

In another embodiment, the invention provides a method for treating ing a subject having or at risk of having a virally mediated disorder by administering to the subject a therapeutically effective amount of a nucleic acid encoding an antigenic protein and an effective amount of an ODN according to the present invention, especially an oligonucleotide containing at least one unmethylated CpdI/dU dinucleotide.

In further embodiment, the invention provides a method for treating a subject having or at risk of having a chronic viral infection by administering to the subject an effective amount of an antigenic polypeptide and an effective amount of an ODN according to the present invention (containing a dI/dU residue), especially an oligonucleotide containing at least one unmethylated CpdI/dU dinucleotide.

In another embodiment, a pharmaceutical composition containing an ODN according to the present invention and a nucleic acid encoding an antigenic protein in a pharmaceutically acceptable carrier is provided.

The invention utilizes polynucleotides encoding the antigenic polypeptides. These polynucleotides include DNA, cDNA and RNA sequences which encode an antigenic polypeptide. Such polynucleotides include naturally occurring, synthetic, and intentionally manipulated polynucleotides. For example, polynucleotide enocing an antigenic polypeptide may be subjected to site-directed mutagenesis, so long as the polypeptide remains antigenic.

The term “polynucleotide” or “nucleic acid sequence” may refer to a polymeric form of nucleotides at least 10 bases in length. By “isolated polynucleotide” is meant a polynucleotide that is not immediately contiguous with both of the coding sequences with which it is immediately contiguous (one on the 5′ end and one on the 3′ end) in the naturally occurring genome of the organism from which it is derived. The term therefore includes, for example, a recombinant DNA which is incorporated into a vector; into an autonomously replicating plasmid or virus; or into the genomic DNA of a prokaryote or eukaryote, or which exists as a separate molecule (e.g. a cDNA) independent of other sequences. The nucleotides of the invention can be ribonucleotides, deoxyribonucleotides, or modified forms of either nucleotide. The term includes single and double forms of DNA.

In the present invention, the polynucleotide sequences encoding an antigenic polypeptide may be inserted into an expression vector. The term “expression vector” refers to a plasmid, virus or other vehicle known in the art that has been manipulated by insertion or incorporation of the genetic sequences encoding the antigenic polypeptide.

Polynucleotide sequence which encode the antigenic polypeptide can be operatively linked to expression control sequences. “Operatively linked” refers to a juxtaposition wherein the components so described are in a relationship permitting them to function in their intended manner. An expression control sequenc maintenance of the correct reading frame of that gene to permit proper translation of MRNA, and stop codons. The term “control sequences” is intended to included, at a minimum, components whose presence can influence expression, and can also include additional components whose presence is advantageous, for example, leader sequences and fusion partner sequences. Expression control sequences can include a promoter.

By “promoter” is meant minimal sequence sufficient to direct transcription. Also included in the invention are those promoter elements which are sufficient to render promoter-dependent gene expression controllable for cell-type specific, tissue-specific, or inducible by external signals or agents; such elements may be located in the 5′ or 3′ regions ofthe gene. Both constitutive and inducible promoters, are included in the invention. Promoters derived from the genome of mammalian cells (e.g., metallothionein promoter) or from mammalian viruses (e.g., the retrovirus long terminal repeat; the adenovirus late promoter; the vaccinia virus 7.5K promoter) may be used. Promoters produced by recombinant DNA or synthetic techniques may also be used to provide for transcription of the nucleic acid sequences of the invention.

The present invention further relates to methods and products for inducing a synergistic immune response using. a combination of an ODN according to the present invention (containing dI/dU residue), especially a CpdI/dU oligonucleotide, and a cytokine. In one aspect the invention is a method for stimulating an immune response in a subject. The method includes the steps of administering to a subject exposed to an antigen an effective amount for inducing a synergistic antigen specific immune response of an immunopotentiating cytokine and an ODN according to the present invention, especially ODNs having a sequence including at least the following formula: 5′X1C(dI/dU)X2 3′ wherein the oligonucleotide includes at least 8 nucleotides wherein C and dI/dU are unmethylated and wherein X1 and X2 are nucleotides.

The cytokine may, for instance be GM-CSF, IL-3, IL-5, IL-12, or interferon-y. The immunopotentiating cytokine may also be an antigen-cytokine fusion protein. In a preferred embodiment the antigen-cytokine fusion protein is an antigen-GM-CSF fusion protein.

The antigen may be any type of antigen known in the art. In one embodiment the antigen is a selected from the group consisting of a tumor-antigen, a microbial antigen, and an allergen. Preferably the antigen is a tumor antigen. In this embodiment the subject may have a neoplastic disorder. In other embodiments the. antigen is a viral antigen and the subject has or is at risk of having a viral infection.

In some embodiments the antigen is administered to the subject in conjunction with the ODN and the immunopotentiating cytokine. In other embodiments the subject is passively exposed to the antigen.

In other aspects the invention is a composition of an effective amount for synergistically activating a dendritic cell of an immunostimulatory ODN according to the present invention, especially an ODN having a sequence including at least the following formula: 5′X1 C(dI/dU)X2 3′ wherein the oligonucleotide includes at least 8 nucleotides wherein C and dI/dU are unmethylated and wherein X1 and X2 are nucleotides; and a cytokine selected from the group consisting of GM-CSF, IL-4, TNFa, Flt3 ligand, and IL-3. Preferably the cytokine is GM-CSF.

The composition may also include an antigen. In some embodiments the antigen is selected from the group consisting of a cancer antigen, a microbial antigen, and an allergen.

A method for activating a dendritic cell is provided according to another aspect of the invention. The method includes the step of contacting a dendritic cell exposed to an antigen with an effective amount for synergistically activating a dendritic cell of an immunopotentiating cytokine and a dI/dU containing ODN, especially an immunostimulatory CpdI/dU oligonucleotide having a sequence including at least the following formula: 5′X1 C(dI/dU)X2 3, wherein the oligonucleotide includes at least 8 nucleotides wherein C and dI/dU are unmethylated and wherein X1 and X2 are nucleotides.

The cytokine may, for instance be GM-CSF, IL-3, IL-5, IL-12, or interferon-gamma. The immunopotentiating cytokine may also be an antigen-cytokine fusion protein. In a preferred embodiment the antigen-cytokine fusion protein is an antigen-GM-CSF fusion protein.

The antigen may be any type of antigen known in the att. In one embodiment the antigen is a selected from the group consisting of a tumor antigen, a microbial antigen, and an allergen. Preferably the antigen is a tumor antigen. In this embodiment the subject may have a neoplastic disorder. In other embodiments the antigen is a viral antigen and the subject has or is at risk of having a viral infection.

According to another aspect the invention is a method for treating a subject having a neoplastic disorder. The method includes the step of administering to the tumor of a subject having a neoplastic disorder an ODN according to the present invention, especially an immunostimulatory CpdI/dU oligonucleotide having a sequence including at least the following formula: 5′ X1 C(dI/dU)X2 3′ wherein the oligonucleotide includes at least 8 nucleotides wherein C and dI/dU are unmethylated and wherein X1 and X2 are nucleotides, and an immunopotentiating cytokine in an amount effective for synergistically increasing survival time of the subject with respect to a subject administered the ODN, especially the immunostimulatory CpdI/dU oligonucleotide, or the immunopotentiating cytokine alone.

Preferably the tumor is selected from the group consisting of a tumor of the brain, lung, ovary, breast, prostate, colon, skin, and blood. In one embodiment the ODN, especially the immunostimulatory CpdI/dU oligonucleotide, and the immunopotentiating cytokine are injected directly into the tumor.

A contraceptive method is provided in another aspect of the invention. The method involves the step of administering to a subject an antigen, an immunopotentiating cytokine and an ODN according to the present invention (containing dI/dU), especially an immunostimulatory CpdI/dU oligonucleotide having a sequence including at least the following formula: 5×X1 C(dI/dU)X2 3′ wherein the oligonucleotide includes at least 8 nucleotides wherein C and dI/dU are unmethylated and wherein X1 and X2 are nucleotides, wherein the antigen is an antigen selected from the group consisting of a gonadal cell antigen and an antigen from a cytokine or hormone required for the maintenance of a gonadal cell.

An “antigen” as used herein is a molecule capable of provoking an immune response. Antigens include but are not limited to cells, cell extracts, polysaccharides, polysaccharide conjugates, lipids, glycolipids, carbohydrate, peptides, proteins, viruses, and viral extracts.

The term antigen broadly includes any type of molecule which is recognized by a host immune system as being foreign. Antigens include but are not limited to cancer antigens, microbial antigens, and allergens.

The methods of the invention are useful for treating cancer by stimulating an antigen specific immune response against a cancer antigen. A “cancer antigen” as used herein is a compound, such as a peptide, associated with a tumor or cancer cell surface and which is capable of provoking an immune response when expressed on the surface of an antigen presenting cell in the context of an MHC molecule. Cancer antigens can be prepared from cancer cells either by preparing crude extracts of cancer cells by partially purifying the antigens, by recombinant technology, or by de novo synthesis of known antigens. Cancer antigens include antigens that are immunogenic portions of or are a whole tumor or cancer. Such antigens can be isolated or prepared recombinately or by any other means known in the art. Cancers or tumors include but are not limited to biliary tract cancer; brain cancer; breast cancer; cervical cancer; choriocarcinoma; colon cancer; endometrial cancer; esophageal cancer; gastric cancer; intraepithelial neoplasms; lymphomas; liver cancer; lung cancer (e. g. small cell and non-small cell); melanoma; neuroblastomas; oral cancer; ovarian cancer; pancreas cancer; prostate cancer; rectal cancer; sarcomas; skin cancer; testicular cancer; thyroid cancer; and renal cancer, as well as other carcinomas and sarcomas.

Tumors are antigenic and can be sensitive to immunological destruction. The term “tumor” is usually equated with neoplasm, which literally means “new growth” and is used interchangeably with “cancer. A “neoplastic disorder” is any disorder associated with cell proliferation, specifically with a neoplasm. A “neoplasm” is an abnormal mass of tissue that persists and proliferates after withdrawal of the carcinogenic factor that initiated its appearance. There are two types of neoplasms, benign and malignant. Nearly all benign tumors are encapsulated and are noninvasive; in contrast, malignant tumors are almost never encapsulated but invade adjacent tissue by infiltrative destructive growth. This infiltrative growth can be followed by tumor cells implanting at sites discontinuous with the original tumor. The method of the invention can be used to treat neoplastic disorders in humans, including but not limited to: sarcoma, carcinoma, fibroma, lymphoma, melanoma, neuroblastoma, retinoblastoma, and glioma as well as each of the other tumors described herein.

The invention can also be used to treat cancer and tumors in non human subjects. Cancer is one of the leading causes of death in companion animals (i. e., cats and dogs).

Cancer usually strikes older animals which, in the case of house pets, have become integrated into the family. Forty-five % of dogs older than 10 years of age, are likely to succomb to the disease. The most common treatment options include surgery, chemotherapy and radiation therapy. Others treatment modalities which have been used with some success are laser therapy, cryotherapy, hyperthermia and immunotherapy. The choice of treatment depends on type of cancer and degree of dissemination. Unless the malignant growth is confined to a discrete area in the body, it is difficult to remove only malignant tissue without also affecting normal cells.

Malignant disorders commonly diagnosed in dogs and cats include but are not limited to lymphosarcoma, osteosarcoma, mammary tumors, mastocytoma, brain tumor, melanoma, adenosquamous carcinoma, carcinoid lung tumor, bronchial gland tumor, bronchiolar adenocarcinoma, fibroma, myxochondroma, pulmonary sarcoma, neurosarcoma, osteoma, papilloma, retinoblastoma, Ewing's sarcoma, Wilms tumor, Burkitt's lymphoma, microglioma, neuroblastoma, osteoclastoma, oral neoplasia, fibrosarcoma, osteosarcoma and rhabdomyosarcoma. Other neoplasias in dogs include genital squamous cell carcinoma, transmissable veneral tumor, testicular tumor, seminoma, Sertoli cell tumor, hemangiopericytoma, histiocytoma, chloroma (granulocytic sarcoma), corneal papilloma, corneal squamous cell carcinoma, hemangiosarcoma, pleural mesothelioma, basal cell tumor, thymoma, stomach tumor, adrenal gland carcinoma, oral papillomatosis, hemangioendothelioma and cystadenoma. Additional malignancies diagnosed in cats include follicular lymphoma, intestinal lymphosarcoma, fibrosarcoma and pulmonary squamous cell carcinoma. The ferret, an ever-more popular house pet is known to develop insulinoma, lymphoma, sarcoma, neuroma, pancreatic islet cell tumor, gastric MALT lymphoma and gastric adenocarcinoma.

Neoplasias affecting agricultural livestock include leukemia, hemangiopericytoma and bovine ocular neoplasia (in cattle); preputial fibrosarcoma, ulcerative squamous cell carcinoma, preputial carcinoma, connective tissue neoplasia and mastocytoma (in horses); hepatocellular carcinoma (in swine); lymphoma and pulmonary adenomatosis (in sheep); pulmonary sarcoma, lymphoma, Rous sarcoma, reticulendotheliosis, fibrosarcoma, nephroblastoma, B-cell lymphoma and lymphoid leukosis (in avian species); retinoblastoma, hepatic neoplasia, lymphosarcoma (lymphoblastic lymphoma), plasmacytoid leukemia and swimbladder sarcoma (in fish), caseous lumphadenitis (CLA): chronic, infectious, contagious disease of sheep and goats caused by the bacterium Corynebacterium pseudotuberculosis, and contagious lung tumor of sheep caused by jaagsiekte.

In the method of the invention, dI/dU containing oligonucleotides are used with an immunopotentiating cytokine. “Immunopotentiating cytokines” are those molecules and compounds which stimulate the humoral and/or cellular immune response. The term “cytokine” is used as a generic name for a diverse group of soluble proteins and peptides which act as humoral regulators at nano-to picomolar concentrations and which, either under normal or pathological conditions, modulate the functional activities of individual cells and tissues. These proteins also mediate interactions between cells directly and regulate processes taking place in the extracellular environment. Examples of cytokines include, but are not limited to IL-1, IL-2, IL-4, IL-5, IL-6, IL-7, IL-10, IL-12, IL-15, granulocyte-macrophage colony stimulating factor (G-MCSF), granulocyte colony stimulating factor (GCSF), interferon-y (y-INF), tumor necrosis factor (TNF), TGF- , FLT-3 ligand, and CD40 ligand.

FLT3 ligand is a class of compounds described in EP0627487A2 and W094/28391. A human FLT3 ligand cDNA was deposited with the American Tissue Type Culture Collection, Rockville, Md., and assigned accession number ATCC 69382. Interleukins (Ils) have been described extensively in the art. GM-CSF is commercially available as sargramostine, leukine (Immunex).

Cytokines play a role in directing the T cell response. Helper (CD4+) T cells orchestrate the immune response of mammals through production of soluble factors that act on other immune system cells, including other T cells. Most mature CD4+ T helper cells express one of two cytokine profiles: Th1 or Th2. Thl cells express IL-3, IL-4, IL-5, IL-6, IL-9, IL-10, IL-13, GM-CSF and low levels of TNF-a. The TH1 subset promotes delayedtype hypersensitivity, cell-mediated immunity, and immunoglobulin class switching to IgG2a.

The Th2 subset induces humoral immunity by activating B cells, promoting antibody production, and inducing class switching to IgG, and IgE.

Tumors can express “tumor-specific antigens” which are antigens that can potentially stimulate apparently tumor-specific immune responses. These antigens can be encoded by normal genes and fall into several categories (1) normally silent genes, (2) differentiation antigens (3) embryonic and fetal antigens, and (4) clonal antigens, which are expressed only on a few normal cells such as the cells from which the tumor originated. Tumor-specific antigens can be encoded by mutant cellular genes, such as oncogenes (e. g., activated ras oncogene), suppressor genes (e. g., mutant p53), fusion proteins resulting from internal deletions or chromosomal translocations. Tumor-specific antigens can also be encoded by viral genes, such as RNA or DNA tumor viruses.

In the treatment of lymphoma, the idiotype of the secreted immunoglobulin serves as a highly specific tumor associated antigen. By “idiotype” is meant the collection of V-region determinants specific to a specific antibody or a limited set of antibodies. In one embodiment, the immunopotentiating cytokine is a protein (a fusion protein) consisting of a specific antigen idiotype secreted by a lymphoma fused to the immunopotentiating cytokine. Methods of producing antigen-cytokine fusion proteins are well known in the art. In one embodiment, the fusion protein is an antigen-GM-CSF fusion protein.

The methods of the invention are also useful for treating infectious diseases. An infectious disease, as used herein, is a disease arising from the presence of a foreign microorganism in the body. dI/dU containing ODNs and immunopotentiating cytokines are used to stimulate an antigen specific immune response which can activate a T or B cell response against an antigen of the microorganism. The methods are accomplished in the same way as described above for the tumor except that the antigen is specific for a microorganism using a microbial antigen. A “microbial antigen” as used herein is an antigen of a microorganism and includes but is not limited to infectious virus, infectious bacteria, and infectious fungi. Such antigens include the intact microorganism as well as natural isolates and fragments or derivatives thereof and also synthetic compounds which are identical to or similar to natural microorganism antigens and induce an immune response specific for that microorganism. A compound is similar to a natural microorganism antigen if it induces an immune response (humoral and/or cellular) to a natural microorganism antigen. Such antigens are used routinely in the art and are well known to those of ordinary skill in the art.

Examples of infectious virus that have been found in humans include but are not limited to: Retroviridae (e. g. human immunodeficiency viruses, such as HIV-1 (also referred to as HTLV-III, LAV or HTLV-III/LAV, or HIV-III; and other isolates, such as HIV-LP; Picornaviridae (e. g. polio viruses, hepatitis A virus; enteroviruses, human Coxsackie viruses, rhinoviruses, echoviruses); Calciviridae (e. g. strains that cause gastroenteritis); Togaviridae (e. g. equine encephalitis viruses, rubella viruses); Flaviridae (e. g. dengue viruses, encephalitis viruses, yellow fever viruses); Coronoviridae (e. g. coronaviruses); Rhabdoviradae (e. g. vesicular stomatitis viruses, rabies viruses); Coronaviridae (e. g. Coronaviruses); Rhabdoviridae (e. g. vesicular stomatitis viruses, rabies viruses); Filoviridae (e. g. ebola viruses); Paramyxoviridae (e. g. parainfluenza viruses, mumps virus, measles virus, respiratory syncytial virus); Orthomyxoviridae (e. g. influenza viruses); Bungaviridae (e. g. Hantaan viruses, bunga viruses, phleboviruses and Nairo viruses); Arena viridae (hemorrhagic fever viruses); Reoviridae (e. g. reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxviridae (variola viruses, vaccinia viruses, pox viruses); and Iridoviridae (e. g. African swine fever virus); and unclassified viruses (e. g. the etiological agents of Spongiform encephalopathies, the agent of delta hepatitis (thought to be a defective satellite of hepatitis B virus), the agents of non-A, non-B hepatitis (class 1=internally transmitted; class 2=parenterally transmitted (i. e. Hepatitis C); Norwalk and related viruses, and astroviruses).

Both gram negative and gram positive bacteria serve as antigens in vertebrate animals. Such gram positive bacteria include, but are not limited to Pasteurella species, Staphylococci species, and Streptococcus species. Gram negative bacteria include, but are not limited to, Escherichia coli, Pseudomonas species, and Salmonella species. Specific examples of infectious bacteria include but are not limited to: Helicobacter pyloris, Borelia burgdorferi, Legionella pneumophilia, Mycobacteria sps (e. g. M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae. Bacillus antracis, corynebacterium diphtheriae, corynebacterium sp., Erysipelothrix rhusiopathiae, Clostridium perfringers, Clostridium tetani, Enterobacter aerogenes. Klebsiella pneumoniae, Pasturella multocida, Bacteroides sp., Fusobacterium nucleatum. Streptobacillus moniliformis, Treponema pallidium, Treponema pertenue, Leptospira, Rickettsia, and Actinomyces israelli.

Examples of infectious fungi include: Cryptococcus neoformans, Histoplasma capsulatum, Coccidioides immitis, Blastomyces dermatitidis, Chlamydia trachomatis, Candida albicans. Other infectious organisms (i. e., protists) include: Plasmodium such as Plasmodium falciparum, Plasmodium malariae, Plasmodium ovale, and Plasmodium vivax and Toxoplasma gondii.

The methods of the invention are also useful for treating allergic diseases. The methods are accomplished in the same way as described above for the tumor immunotherapy and treatment of infectious diseases except that the antigen is specific for an allergen.

Currently, allergic diseases are generally treated by the injection of small doses of antigen followed by subsequent increasing dosage of antigen. It is believed that this procedure. produces a memory immune response to prevent further allergic reactions. These methods, however, are associated with the risk of side effects such as an allergic response. The methods of the invention avoid these problems.

“Asthma”—refers to a disorder of the respiratory system characterized by inflammation, narrowing of the airways and increased reactivity of the airways to inhaled agents. Asthma is frequently, although not exclusively associated with atopic or allergic symptoms.

An “allergen” refers to a substance (antigen) that can induce an allergic or asthmatic response in a susceptible subject. The list of allergens is enormous and can include pollens, insect venoms, animal dander dust, fungal spores and drugs (e. g. penicillin). Examples of natural, animal and plant allergens include but are not limited to proteins specific to the following genuses: Canine (Canis familiaris); Dermatophagoides (e. g. Dermatophagoides farinae); Felis (Felis domesticus); Ambrosia (Ambrosia artemiisfolia; Lolium (e. g. Lolium perenne or Lolium multiflorum); Cryptomeria (Cryptomeria japonica); Alternaria (Alternaria alternata); Alder; Alnus (Alnus gultinoasa); Betula (Betula verrucosa); Quercus (Quercus alba); Olea (Olea europa); Artemisia (Artemisia vulgaris); Plantago (e. g. Plantago lanceolata); Parietaria (e. g. Parietaria officinalis or Parietaria judaica); Blattella (e. g. Blattella germanica); Apis (e. g. Apis multiflorum); Cupressus (e. g. Cupressus sempervirens, Cupressus arizonica and Cupressus macrocarpa); Juniperus (e. g. Juniperus sabinoides, Juniperus virginiana, Juniperus communis and Juniperus ashei); Thuya (e. g. Thuya orientalis); Chamaecyparis (e. g. Chamaecyparis obtusa); Periplaneta (e. g. Periplaneta americana); Agropyron (e. g. Agropyron repens); Secale (e. g. Secale cereale); Triticum (e. g. Triticum aestivum); Dactylis (e. g. Dactylis glomerata); Festuca (e. g. Festuca elatior); Poa (e. g. Poa pratensis or Poa compressa); Avena (e. g. Avena sativa); Holcus (e. g. Holcus lanatus); Anthoxanthum (e. g. Anthoxanthum odoratum); Arrhenatherum (e. g. Arrhenatherum elatius); Agrostis (e. g. Agrostis alba); Phleum (e. g. Phleum pratense); Phalaris (e. g. Phalaris arundinacea); Paspalum (e. g. Paspalum notatum); Sorghum (e. g. Sorghum halepensis); and Bromus (e. g. Bromus inermis).

An “allergy” refers to acquired hypersensitivity to a substance (allergen). Allergic conditions include but are not limited to eczema, allergic rhinitis or coryza, hay fever, bronchial asthma, urticaria (hives) and food allergies, and other atopic conditions. A subject having an allergic reaction is a subject that has or is at risk of developing an allergy. Allergies are generally caused by IgE antibody generation against harmless allergens.

The cytokines: that are induced by the dI/dU oligonucleotides are predominantly of a class called “Th1” which is most marked by a cellular immune response and is associated with IL-12 and IFN-gamma and production of IgG2a antibody. The other major type of immune response is termed as Th2 immune response, which is associated with more of an IgG I antibody immune response and with the production of IL-4, IL-5 and IL-10. In general, it appears that allergic diseases are mediated by Th2 type immune responses and autoimmune diseases by Th1 immune response. Based on the ability of the combination of dI/dU oligonucleotides especially CpdI/dU oligonucleotides, and immunopotentiating cytokine to shift the immune response in a subject from a Th2 (which is associated with production of IgE antibodies and allergy and is produced in response to GM-CSF alone) to a Th1 response (which is protective against allergic reactions), an effective dose of a dI/dU oligonucleotide and immunopotentiating cytokine can be administered to a subject to treat or prevent an allergy.

dI/dU oligonucleotides, especially CpdI/dU oligonucleotides, combined with immunopotentiating cytokines may also have significant therapeutic utility in the treatment of asthma. Th2 cytokines, especially IL-4 and IL-5 are elevated in the airways of asthmatic subjects. These cytokines promote important aspects of the asthmatic inflammatory response, including IgE isotope switching, eosinophil chemotaxis and activation and mast cell growth. Th1 cytokines, especially IFN-y and IL-12, can suppress the formation of Th2 clones and production of Th2 cytokines. “Asthma” refers to a disorder of the respiratory system characterized by inflammation, narrowing of the airways and increased reactivity of the airways to inhaled agents. Asthma is frequently, although not exclusively associated with atopic or allergic symptoms.

Thus the present invention contemplates the use of dI/du containing oligonucleotides and immunopotentiating cytokines to induce an antigen specific immune response in human and non-human animals. As discussed above, antigens include infectious microbes such as virus, bacteria and fungi and fragments thereof, derived from natural sources or synthetically.

Infectious virus of both human and non-human vertebrates, include retroviruses, RNA viruses and DNA viruses. This group of retroviruses includes both simple retroviruses and complex retroviruses. The simple retroviruses include the subgroups of B-type retroviruses, C-type retroviruses and D-type retroviruses. An example of a B-type retrovirus is mouse mammary tumor virus (MMTV). The C-type retroviruses include subgroups C-type group A (including Rous sarcoma virus (RSV), avian leukemia virus (ALV), and avian myeloblastosis virus (AMV)) and C-type group B (including murine leukemia virus (MLV), feline leukemia virus (FeLV), murine sarcoma virus (MSV), gibbon ape leukemia virus (GALV), spleen necrosis virus (SNV), reticuloendotheliosis virus (RV) and simian sarcoma virus (SSV)) . The D-type retroviruses include Mason-Pfizer monkey virus (MPMV) and simian retrovirus type 1 (SRV-1). The complex retroviruses include the subgroups of lentiviruses, T-cell leukemia viruses and the foamy viruses. Lentiviruses include HIV-1, but also include HIV-2, SIV, Visna virus, feline immunodeficiency virus (FIV), and equine infectious anemia virus (EIAV). The T-cell leukemia viruses include HTLV-1, HTLV-II, simian T-cell leukemia virus (STLV), and bovine leukemia virus (BLV). The foamy viruses include human foamy virus (HFV), simian foamy virus (SFV) and bovine foamy virus (BFV).

Examples of other RNA viruses that are antigens in vertebrate animals include, but are not limited to, the following: members of the family Reoviridae, including the genus Orthoreovirus (multiple serotypes of both mammalian and avian retroviruses), the genus Orbivirus (Bluetongue virus, Eugenangee virus, Kemerovo virus, African horse sickness virus, and Colorado Tick Fever virus), the genus Rotavirus (human rotavirus, Nebraska calf diarrhea virus, murine rotavirus, simian rotavirus, bovine or ovine rotavirus, avian rotavirus); the family Picornaviridae, including the genus Enterovirus (poliovirus, Coxsackie virus A and B, enteric cytopathic human orphan (ECHO) viruses, hepatitis A virus, Simian enteroviruses, Murine encephalomyelitis (ME) viruses, Poliovirus muris, Bovine enteroviruses. Porcine enteroviruses, the genus Cardiovirus (Encephalomyocarditis virus (EMC), Mengovirus), the genus Rhinovirus (Human rhinoviruses including at least 113 subtypes; other rhinoviruses), the genus Apthovirus (Foot and Mouth disease (FMDV); the family Calciviridae, including Vesicular exanthema of swine virus, San Miguel sea lion virus, Feline picornavirus and Norwalk virus; the family Togaviridae, including the genus Alphavirus (Eastern equine encephalitis virus, Semliki forest virus, Sindbis virus, Chikungunya Virus, O'Nyong-Nyong virus, Ross river virus, Venezuelan equine encephalitis virus, Western equine encephalitis virus), the genus Flavirius (Mosquito borne yellow fever virus, Dengue virus, Japanese encephalitis virus, St. Louis encephalitis virus, Murray Valley encephalitis virus, West Nile virus, Kunjin virus, Central European tick borne virus, Far Eastern tick borne virus, Kyasanur forest virus, Louping III virus, Powassan virus, Omsk hemorrhagic fever virus), the genus Rubivirus (Rubella virus), the genus Pestivirus (Mucosal disease virus, Hog cholera virus, Border disease virus); the family Bunyaviridae, including the genus Bunyvirus (Bunyamwera and related viruses, California encephalitis group viruses), the genus Phlebovirus (Sandfly fever Sicilian virus, Rift Valley fever virus), the genus Nairovirus (Crimean-Congo hemorrhagic fever virus, Nairobi sheep disease virus), and the genus Uukuvirus (Uukuniemi and related viruses); the family Orthomyxoviridae, including the genus Influenza virus (Influenza virus type A, many human subtypes); Swine influenza virus, and Avian and Equine Influenza viruses; influenza type B (many human subtypes), and influenza type C (possible separate genus); the family paramyxoviridae, including the genus Paramyxovirus (Parainfluenza virus type 1, Sendai virus, Hemadsorption virus, Parainfluenza viruses types 2 to 5, Newcastle Disease Virus, Mumps virus), the genus Morbillivirus (Measles virus, subacute sclerosing panencephalitis virus, distemper virus, Rinderpest virus), the genus Pneumovirus (respiratory syncytial virus (RSV), Bovine respiratory syncytial virus and Pneumonia virus of mice); forest virus, Sindbis virus, Chikungunya virus, O'Nyong-Nyong virus, Ross river virus, Venezuelan equine encephalitis virus, Western equine encephalitis virus), the genus Flavirius (Mosquito borne yellow fever virus, Dengue virus, Japanese encephalitis virus, St. Louis encephalitis virus, Murray Valley encephalitis virus, West Nile virus, Kunjin virus, Central European tick borne virus, Far Eastern tick borne virus, Kyasanur forest virus, Louping III virus, Powassan virus, Omsk hemorrhagic fever virus), the genus Rubivirus (Rubella virus), the genus Pestivirus (Mucosal disease virus, Hog cholera virus, Border disease virus); the family Bunyaviridae, including the genus Bunyvirus (Bunyamwera and related viruses, California encephalitis group viruses), the genus Phlebovirus (Sandfly fever Sicilian virus, Rift Valley fever virus), the genus Nairovirus (Crimean-Congo hemorrhagic fever virus, Nairobi sheep disease virus), and the genus Uukuvirus (Uukuniemi and related viruses); the family Orthomyxoviridae, including the genus Influenza virus (Influenza virus type A, many human subtypes); Swine influenza virus, and Avian and Equine Influenza viruses; influenza type B (many human subtypes), and influenza type C (possible separate genus); the family paramyxoviridae, including the genus Paramyxovirus (Parainfluenza virus type 1, Sendai virus, Hemadsorption virus, Parainfluenza viruses types 2 to 5, Newcastle Disease Virus, Mumps virus), the genus Morbillivirus (Measles virus, subacute sclerosing panencephalitis virus, distemper virus, Rinderpest virus), the genus Pneumovirus (respiratory syncytial virus (RSV), Bovine respiratory syncytial virus and Pneumonia virus of mice); the family Rhabdoviridae, including the genus Vesiculovirus (VSV), Chandipura virus, Flanders-Hart Park virus), the genus Lyssavirus (Rabies virus), fish Rhabdoviruses, and two probable Rhabdoviruses (Marburg virus and Ebola virus) ; the family Arenaviridae, including Lymphocytic choriomeningitis virus (LCM), Tacaribe virus complex, and Lassa virus; the family Coronoaviridae, including Infectious Bronchitis Virus (IBV), Mouse Hepatitis virus, Human enteric corona virus, and Feline infectious peritonitis (Feline coronavirus).

Illustrative DNA viruses that are antigens in vertebrate animals include, but are not limited to: the family Poxviridae, including the genus Orthopoxvirus (Variola major, Variola minor, Monkey pox Vaccinia, Cowpox, Buffalopox, Rabbitpox, Ectromelia), the genus Leporipoxvirus (Myxoma, Fibroma), the genus Avipoxvirus (Fowlpox, other avian poxvirus), the genus Capripoxvirus (sheeppox, goatpox), the genus Suipoxvirus (Swinepox), the genus Parapoxvirus (contagious postular dermatitis virus, pseudocowpox, bovine papular stomatitis virus); the family Iridoviridae (African swine fever virus, Frog viruses 2 and 3, Lymphocystis virus of fish); the family Herpesviridae, including the alpha-Herpesviruses (Herpes Simplex Types 1 and 2, Varicella-Zoster, Equine abortion virus, Equine herpes virus 2 and 3, pseudorabies virus, infectious bovine keratoconjunctivitis virus, infectious bovine rhinotracheitis virus, feline rhinotracheitis virus, infectious laryngotracheitis virus) the Beta-herpesviruses (Human cytomegalovirus and cytomegaloviruses of swine, monkeys and rodents); the gamma-herpesviruses (Epstein-Barr virus (EBV), Marek's disease virus, Herpes saimiri, Herpesvirus ateles, Herpesvirus sylvilagus, guinea pig herpes virus, Lucke tumor virus); the family Adenoviridae, including the genus Mastadenovirus (Human subgroups A, B, C, D, E and ungrouped; simian adenoviruses (at least 23 serotypes), infectious canine hepatitis, and adenoviruses of cattle, pigs, sheep, frogs and many other species, the genus Aviadenovirus (Avian adenoviruses); and non-cultivatable adenoviruses; the family Papoviridae, including the genus Papillomavirus (Human papilloma viruses, bovine papilloma viruses, Shope rabbit papilloma virus, and various pathogenic papilloma viruses of other species), the genus Polyomavirus (polyomavirus, Simian vacuolating agent (SV-40), Rabbit vacuolating agent (RKV), K virus, BK virus, JC virus, and other primate polyoma viruses such as Lymphotrophic papilloma virus); the family Parvoviridae including the genus Adeno-associated viruses, the genus Parvovirus (Feline panleukopenia virus, bovine parvovirus, canine parvovirus, Aleutian mink disease virus, etc). Finally, DNA viruses may include viruses which do not fit into the above families such as Kuru and Creutzfeldt-Jacob disease viruses and chronic infectious neuropathic agents (CHINA virus).

In addition to the use of the combination of dI/dU oligonucleotides and immunopotentiating cytokines to induce an antigen specific immune response in humans, the methods of the preferred embodiments are particularly well suited for treatment of birds such as hens, chickens, turkeys, ducks, geese, quail, and pheasant. Birds are prime targets for many types of infections. Hatching birds are exposed to pathogenic microorganisms shortly after birth. Although these birds are initially protected against pathogens by maternal derived antibodies, this protection is only temporary, and the bird's own immature immune system must begin to protect the bird against the pathogens. It is often desirable to prevent infection in young birds when they are most susceptible. It is also desirable to prevent against infection in older birds, especially when the birds are housed in closed quarters, leading to the rapid spread of disease.

Thus, it is desirable to administer the dI/dU oligonucleotide and the immunopotentiating cytokine of the invention to birds to enhance an antigen-specific immune response when antigen is present.

An example of a common infection in chickens is chicken infectious anemia virus (CIAV). CIAV was first isolated in Japan in 1979 during an investigation of a Marek's disease vaccination break. Since that time, CIAV has been detected in commercial poultry in all major poultry producing countries. CIAV infection results in a clinical disease, characterized by anemia, hemorrhage and immunosuppression, in young susceptible chickens. Atrophy of the thymus and of the bone marrow and consistent lesions of CIAV-infected chickens are also characteristic of CIAV infection. Lymphocyte depletion in the thymus, and occasionally in the bursa of Fabricius, results in immunosuppression and increased susceptibility to secondary viral, bacterial, or fungal infections which then complicate the course of the disease. The immunosuppression may cause aggravated disease after infection with one or more of Marek's disease virus (MDV), infectious bursal disease virus, reticuloendotheliosis virus, adenovirus, or reovirus. It has been reported that pathogenesis of MDV is enhanced by CIAV. Further, it has been reported that CIAV aggravates the signs of infectious bursal disease. Chickens develop an age resistance to experimentally induced disease due to CAA. This is essentially complete by the age of 2 weeks, but older birds are still susceptible to infection. However, if chickens are dually infected with CAA and an immunosuppressive agent (IBDV, MDV etc.) age resistance against the disease is delayed. Characteristics of CIAV that may potentiate disease transmission include high resistance to environmental inactivation and some common disinfectants. The economic impact of CIAV infection on the poultry industry is clear from the fact that 10% to 30% of infected birds in disease outbreaks die.

Vaccination of birds, like other vertebrate animals can be performed at any age. Normally, vaccinations are performed at up to 12 weeks of age for a live microorganism and between 14-18 weeks for an inactivated microorganism or other type of vaccine. For in ovo vaccination, vaccination can be performed in the last quarter of embryo development. The composition may be administered subcutaneously, by spray, orally, intraocularly, intratracheally, nasally, in ovo or by other methods described herein. Thus, the CpdI/dU oligonucleotide and immunopotentiating cytokine of the invention can be administered to birds and other non human vertebrates using routine vaccination schedules and the antigen is administered after an appropriate time period as described herein.

Cattle and livestock are also susceptible to infection. Disease which affect these animals can produce severe economic losses, especially amongst cattle. The methods of the invention can be used to protect against infection in livestock, such as cows, horses, pigs, sheep, and goats.

Cows can be infected by bovine viruses. Bovine viral diarrhea virus (BVDV) is a small enveloped positive-stranded RNA virus and is classified, along with hog cholera virus (HOCV) and sheep border disease virus (BDV), in the pestivirus genus. Although, Pestiviruses were previously classified in the Togaviridae family, some studies have suggested their reclassification within the Flaviviridae family along with the flavivirus and hepatitis C virus (HCV) groups.

A subject at risk of developing a cancer can also be treated according to the methods of the invention, by passive or active exposure to antigen following dI/dU and immunopotentiating cytokine. A subject at risk of developing a cancer is one who is who has a high probability of developing cancer. These subjects include, for instance, subjects having a genetic abnormality, the presence of which has been demonstrated to have a correlative relation to a higher likelihood of developing a cancer and subjects exposed to cancer causing agents such as tobacco. asbestos, or other chemical toxins. When a subject at risk of developing a cancer is treated with dI/dU and immunopotentiating cytokine on a regular basis, such as monthly, the subject will be able to recognize and produce an antigen specific immune response. If a tumor begins to form in the subject, the subject will develop a specific immune response against one or more of the tumor antigens. This aspect of the invention is particularly advantageous when the antigen to which the subject will be exposed is unknown. For instance, in soldiers at risk of exposure to biowarfare, it is generally not known what biological weapon to which the soldier might be exposed.

The antigen may be delivered to the immune system of a subject alone or with a carrier.

For instance, colloidal dispersion systems may be used to deliver antigen to the subject. As used herein, a “colloidal dispersion system” refers to a natural or synthetic molecule, other than those derived from bacteriological or viral sources, capable of delivering to and releasing the antigen in a subject. Colloidal dispersion systems include macromolecular complexes, nanocapsules, microspheres, beads, and lipid-based systems including oil-in-water emulsions, micelles, mixed micelles, and liposomes. A preferred colloidal system of the invention is a liposome. Liposomes are artificial membrane vessels which are useful as a delivery vector in vivo or in vitro. It has been shown that large unilamellar vessels (LUV), which range in size from u can encapsulate large macromolecules within the aqueous interior and these macromolecules can be delivered to cells in a biologically active form.

Lipid formulations for transfection are commercially available from QIAGEN, for example as EFFECTENETM (a non-liposomal lipid with a special DNA condensing enhancer) and SUPER-FECTTM (a novel acting dendrimeric technology) as well as Gibco BRL, for example, as LIPOFECTINTM and LIPOFECTACETM, which are formed of cationic lipids such as N-[1-(2,3dioleyloxy)-propyl]-N,N,N-trimeth-ylammonium chloride (DOTMA) and dimethyl dioctadecylammonium bromide (DDAB). Methods for making liposomes are well known in the art and have been described in many publications.

It is envisioned that the antigen may be delivered to the subject in a nucleic acid molecule which encodes for the antigen such that the antigen must be expressed in vivo. In these embodiments of the invention the nucleic acids molecule may also include a dI/dU, especially a CpdI/dU, dinucleotide within the sequence of the nucleic acid. But in this case the nucleic acid molecule does not take the place of the dI/dU oligonucleotide. The antigen must be administered in conjunction with a dI/dU oligonucleotide that is separate from the nucleic acid molecule. The nucleic acid encoding the antigen is operatively linked to a gene expression sequence which directs the expression of the antigen nucleic acid within a eukaryotic cell. The “gene expression sequence” is any regulatory nucleotide sequence, such as a promoter sequence or promoter-enhancer combination, which facilitates the efficient transcription and translation of the antigen nucleic acid to which. it is operatively linked. The gene expression sequence may, for example, be a mammalian or viral promoter, such as a constitutive or inducible promoter. Constitutive mammalian promoters include, but are not limited to, the promoters for the following genes: hypoxanthine phosphoribosyl transferase (HPTR), adenosine deaminase, pyruvate kinase, p-actin promoter and other constitutive promoters. Exemplary viral promoters which function constitutively in eukaryotic cells include, for example, promoters from the simian virus, papilloma virus, adenovirus, human immunodeficiency virus (HIV), rous sarcoma virus, cytomegalovirus, the long terminal repeats (LTR) of moloney leukemia virus and other retroviruses, and the thymidine kinase promoter of herpes simplex virus. Other constitutive promoters are known to those of ordinary skill in the art. The promoters useful as gene expression sequences of the invention also include inducible promoters. Inducible promoters are expressed in the presence of an inducing agent. For example, the metallothionein promoter is induced to promote transcription and translation in the presence of certain metal ions. Other inducible promoters are known to those of ordinary skill in the art.

In general, the gene expression sequence shall include, as necessary, 5′non-transcribing and 5′non-translating sequences involved with the initiation of transcription and translation, respectively, such as a TATA box, capping sequence, CAAT sequence, and the like. Especially, such 5′non-transcribing sequences will include a promoter region which includes a promoter sequence for transcriptional control of the operably joined antigen nucleic acid. The gene expression sequences optionally include enhancer sequences or upstream activator sequences as desired.

The antigen nucleic acid is operatively linked to the gene expression sequence. As used herein, the antigen nucleic acid sequence and the gene expression sequence are said to be “operably linked” when they are covalently linked in such a way as to place the expression or transcription and/or translation of the antigen coding sequence under the influence or control of the gene expression sequence. Two DNA sequences are said to be operably linked if induction of a promoter in the 5′gene expression sequence results in the transcription of the antigen sequence and if the nature of the linkage between the two DNA sequences does not (1) result in the introduction of a frame-shift mutation, (2) interfere with the ability of the promoter region to direct the transcription of the antigen sequence, or (3) interfere with the ability of the corresponding RNA transcript to be translated into a protein. Thus, a gene expression sequence would be operably linked to an antigen nucleic acid sequence if the gene expression sequence were capable of effecting transcription of that antigen nucleic acid sequence such that the resulting transcript is translated into the desired protein or polypeptide.

The antigen nucleic acid of the invention may be delivered to the immune system alone or in association with a vector. In its broadest sense, a “vector” is any vehicle capable of facilitating the transfer of the antigen nucleic acid to the cells of the immune system and preferably APCs so that the antigen can be expressed and presented on the surface of an APC.

Preferably, the vector transports the nucleic acid to the immune cells with reduced degradation relative to the extent of degradation that would result in the absence of the vector. The vector optionally includes the above-described gene expression sequence to enhance expression of the antigen nucleic acid in APCs. In general, the vectors useful in the invention include, but are not limited to, plasmids, phagemids, viruses, other vehicles derived from viral or bacterial sources that have been manipulated by the insertion or incorporation of the antigen nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rouse sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

Preferred viral vectors are based on non-cytopathic eukaryotic viruses in which nonessential genes have been replaced with the gene of interest. Non-cytopathic viruses include retroviruses, the life cycle of which involves reverse transcription of genomic. viral RNA into DNA with subsequent proviral integration into host cellular DNA. Retroviruses have been approved for human gene therapy trials. Most useful are those retroviruses that are replication deficient (i. e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in the art.

A preferred virus for certain applications is the adeno-associated virus, a double-stranded DNA virus. The adeno-associated virus can be engineered to be replication-deficient and is capable of infecting a wide range of cell types and species. It further has advantages such as, heat and lipid solvent stability; high transduction frequencies in cells of diverse lineages, including hemopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Other vectors include plasmid vectors. Plasmid vectors have been extensively described in the art and are well-known to those of skill in the art. In the last few years, plasmid vectors have been found to be particularly advantageous for delivering genes to cells in vivo because of their inability to replicate within and integrate into a host genome. These plasmids, however, having a promoter compatible with the host cell, can express a peptide from a gene operatively encoded within the plasmid. Some commonly used plasmids include pBR322, pUC18, pUC19, pRC/CMV, SV40, and pBlueScript. Other plasmids are well-known to those of ordinary skill in the art. Additionally, plasmids may be custom designed using restriction enzymes and ligation reactions to remove and add specific fragments of DNA.

It has recently been discovered that gene carrying plasmids can be delivered to the immune system using bacteria. Modified forms of bacteria such as Salmonella can be transfected with the plasmid and used as delivery vehicles. The bacterial delivery vehicles can be administered to a host subject orally or by other administration means. The bacteria deliver the plasmid to immune cells, e. g. dendritic cells, probably by passing through the gut barrier. High levels of immune protection have been established using this methodology.

Thus, the invention contemplates scheduled administration of dI/dU oligonucleotides and immunopotentiating cytokine. The oligonucleotides may be administered to a subject on a weekly or monthly basis. When a subject is at risk of exposure to an antigen or antigens the dI/dU and immunopotentiating cytokine may be administered on a regular basis to recognize the antigen immediately upon exposure and produce an antigen specific immune response. A subject at risk of exposure to an antigen is any subject who has a high probability of being exposed to an antigen and of developing an immune response to the antigen. If the antigen is an allergen and the subject develops allergic responses to that particular antigen and the subject is exposed to the antigen, i. e., during pollen season, then that subject is at risk of exposure to the antigen.

The CpdI/dU oligonucleotides of the invention are nucleic acid molecules which contain an unmethylated cytosine-deoxyinosine/deoxyuridine dinucleotide sequence (i. e. “CpdI/dU DNA” or DNA containing a 5′ cytosine followed by 3′guanosine and linked by a phosphate bond) and activate the immune system. The CpdI/dU oligonucleotides can be double-stranded or single-stranded. Generally, doublestranded molecules are more stable in vivo, while single-stranded molecules have increased immune activity.

Another use for the ODNs according to the present invention in combination with an immunopotentiating cytokine is the production of a contraceptive method for use in a subject. In this particular embodiment, the subject is preferably mammalian, and preferably nonhuman. The testes and ovaries are “immune privileged,” that is they are separated anatomically from the immune system. In addition, cells in the testes and the ovaries can express fas ligand, which induces apoptosis in activated T cells. The physical separation and the expression of fas ligand both prevent an immune response against the cells in the testes and ovaries. The dI/dU oligonucleotide used in conjunction with an immunopotentiating cytokine can be used to eliminate or substantially reduce the cells in the testes and the ovaries by breaking the immune privilege of these cells, thereby providing a contraceptive means. dI/dU oligonucleotide can be used in conjunction with an immunopotentiating cytokine to break the immune privilege of the cells of the testes and ovaries.

The method is accomplished by administering to a subject an antigen, an immunopotentiating cytokine and an immunostimulatory dI/dU oligonucleotide, wherein the antigen is an antigen selected from the group consisting of a gonadal cell antigen and an antigen from a cytokine or hormone required for the maintenance of a gonadal cell. A “gonadal cell antigen” as used herein is an antigen on the surface of a gonadal cell, e. g., testis or ovary cell. Such antigens are well known to those of skill in the art. Antigens from a cytokine or hormone required for the maintenance of a gonadal cell are also well known in the art. These antigens will cause an immune response against the cytokine or hormone thus causing a loss of gonadal cells.

The dI/dU oligonucleotides are used in one aspect of the invention to induce activation of immune cells and preferably APCs. An APC has its ordinary meaning in the art and includes, for instance, dendritic cells such as immature dendritic cells and precursor and progenitor dendritic cells, as well as mature dendritic cells which are capable of taking up and expressing antigen. Such a population of APC or dendritic cells is referred to as a primed population of APCs or dendritic cells.

Dendritic cells form the link between the innate and the acquired immune system by presenting antigens as well as through their expression of pattern recognition receptors which detect microbial molecules like LPS in their local environment. The combination of immunopotentiating cytokine and dI/dU oligonucleotide showed induction of Th1 specific antibody when immunopotentiating cytokine alone only produced Th2 specific antibody.

Since dendritic cells form the link between the innate and the acquired immune system the ability to activate dendritic cells with dI/dU and immunopotentiating cytokine supports the use of combination dI/dU-immunopotentiating cytokine based strategies for immunotherapy against disorders such as cancer and allergic or infectious diseases. The combination of dI/dU and immunopotentiating cytokine shows synergistic activation of dendritic cells.

The invention relates in one aspect to methods and products for activating dendritic cells for in vitro, ex vivo and in vivo purposes. It was demonstrated according to the invention that the combination of immunopotentiating cytokine and dI/dU oligonucleotide is a potent activator of dendritic cells. Dendritic cells are believed to be essential for the initiation of primary immune responses in immune cells in vivo. It was discovered, according to the invention, that dI/dU oligonucleotides and immunopotentiating cytokine were capable of activating dendritic cells to initiate primary immune responses in T cells, similar to an adjuvant. It was also discovered that when the combination of the dI/dU oligonucleotide and immunopotentiating cytokine is used to activate dendritic cells the production of predominantly IgG2a and less IgG 1 is induced, indicating its propensity to augment the development of Th1 immune responses in vivo. These findings demonstrate the potent adjuvant activity of dI/dU and provide the basis for the use of dI/dU oligonucleotides as immunotherapeutics in the treatment of disorders such as cancer, infectious diseases, and allergy. In one aspect, the invention is a method for activating a dendritic cell by contacting the dendritic cell which is exposed to an antigen with an effective amount for synergistically activating a dendritic cell of an immunopotentiating cytokine and an immunostimulatory dI/dU oligonucleotide.

Dendritic cells efficiently internalize, process, and present soluble specific antigen to which it is exposed. The process of internalizing and presenting antigen causes rapid upregulation of the expression of major histocompatibility complex (MHC) and costimulatory molecules, the production of cytokines, and migration toward lymphatic organs where they are believed to be involved in the activation of T cells.

One specific use for the combination of dI/dU oligonucleotide and immunopotentiating cytokine of the invention is to activate dendritic cells for the purpose of enhancing a specific immune response against cancer antigens. The immune response may be enhanced using ex vivo or in vivo techniques. An “ex vivo” method as used herein is a method which involves isolation of a dendritic cell from a subject, manipulation of the cell outside of the body, and reimplantation of the manipulated cell into a subject. The ex vivo procedure may be used on autologous or heterologous cells, but is preferably used on autologous cells. In preferred embodiments, the dendritic cells are isolated from peripheral blood or bone marrow, but may be isolated from any source of dendritic cells. When the ex vivo procedure is performed to specifically produce dendritic cells active against a specific cancer or other type of antigen, the dendritic cells may be exposed to the antigen in addition to the dI/dU and immunopotentiating cytokine. In other cases the dendritic cell may have already been exposed to antigen but may not be expressing the antigen on the surface efficiently.

Alternatively the dendritic cell may be exposed to the immunopotentiating cytokine and exposed to the antigen, by either direct contact or exposure in the body and then the dendritic cell is returned to the body followed by administration of dI/dU directly to the subject, either systemically or locally. Activation will dramatically increase antigen processing. The activated dendritic cell then presents the cancer antigen on its surface. When returned to the subject, the activated dendritic cell expressing the cancer antigen activates T cells in vivo which are specific for the cancer antigen. Ex vivo manipulation of dendritic cells for the purposes of cancer immunotherapy have been described in several references in the art. The ex vivo activation of dendritic cells of the invention may be performed by routine ex vivo manipulation steps known in the art, but with the use of dI/dU and immunopotentiating cytokine as the activator.

The dendritic cells may also be contacted with dI/dU and immunopotentiating cytokine using in vivo methods. In order to accomplish this, dI/dU and immunopotentiating cytokine are administered directly to a subject in need of immunotherapy. The dI/dU and immunopotentiating cytokine may be administered in combination with an antigen or may be administered alone. In some embodiments, it is preferred that the dI/dU and immunopotentiating cytokine be administered in the local region of the tumor, which can be accomplished in any way known in the art, e. g., direct injection into the tumor, with implants that release the drug combination, etc.

Dendritic cells useful according to the invention may be isolated from any source as long as the cell is capable of being activated by dI/dU and cytokine to produce an active antigen expressing dendritic cell. Several in vivo sources of immature dendritic cells may be used according to the methods of the invention. For instance bone marrow dendritic cells and peripheral blood dendritic cells are both excellent sources of immature dendritic cells that are activated by dI/dU and cytokine. Other sources may easily be determined by those of skill in the art without requiring undue experimentation, by for instance, isolating a primary source of dendritic cells and testing activation by dI/dU in vitro. The invention also encompasses the use of any immature dendritic cells maintained in culture as a cell line as long as the cell is capable of being activated by dI/dU and cytokine. Such cell types may be routinely identified using standard assays known in the art.

Peripheral blood dendritic cells isolated by immunomagnetic cell sorting, which are activated by dI/dU and cytokine, represent a more physiologic cell population of dendritic cells than monocyte derived dendritic cells. Immature dendritic cells comprise approximately 1-3% of the cells in the bone marrow and approximately 10-100 fold less in the peripheral blood. Peripheral blood cells can be collected using devices well-known in the art, e. g., haemonetics model v. 50 apheresis device (Haemonetics, Brain-tree, MA). Red blood cells and neutrophils are removed from the blood by centrifugation. The mononuclear cells located at the interface are isolated. Methods for isolating CD4+ dendritic cells from peripheral blood have been described. In the presence of GM-CSF alone these cells differentiate to dendritic cells with characteristic cellular processes within two days. Differentiation is accompanied by an increase in cell size, granularity and MHC II expression, which can be easily followed using flow cytometry. Freshly isolated dendritic cells cultured in the absence of GM-CSF rapidly undergo apoptosis. Strikingly, in the presence of CpdI/dU oligonucleotides without addition of GM-CSF, both cell survival and differentiation is markedly improved compared to GM-CSF. In the presence of CpdI/dU, dendritic cells form cell clusters which when examined by ultrastructural techniques such as electron microscopy revealed characteristic dense multilamellar intracytoplasmic bodies and multi-vesicular structures, which were not present in dendritic cells incubated with GM-CSF.

The compositions of the invention may be combined, optionally, with a pharmaceutically-acceptable carrier. The term “pharmaceutically-acceptable carrier’ as used herein means one or more compatible solid or liquid filler, diluents or encapsulating substances which are suitable for administration into a human or other animal. The term “carrier” denotes an organic or inorganic ingredient, natural or synthetic, with which the active ingredient is combined to facilitate the application. The components of the pharmaceutical compositions also are capable of being co-mingled with the molecules of the present invention, and with each other, in a manner such that there is no interaction which would substantially impair the desired pharmaceutical efficacy.

The pharmaceutical compositions may contain suitable buffering agents, including: acetic acid in a salt; citric acid in a salt; boric acid in a salt; and phosphoric acid in a salt.

The pharmaceutical compositions also may contain, optionally, suitable preservatives, such as: benzalkonium chloride; chlorobutanol; parabens and thimerosal.

Compositions suitable for parenteral administration conveniently comprise a sterile aqueous preparation of the compositions of the invention, which is preferably isotonic with the blood of the recipient. This aqueous preparation may be formulated according to known methods using suitable dispersing or wetting agents and suspending agents. The sterile injectable preparation also may be a sterile injectable solution or suspension in a non-toxic parenterally-acceptable diluent or solvent, for example, as a solution in 1,3-butane diol.

Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono-or diglycerides. In addition, fatty acids such as oleic acid may be used in the preparation of injectables. Carrier formulation suitable for oral, subcutaneous, intravenous, intramuscular, etc. administrations can be found in Remington's Pharmaceutical Sciences, Mack Publishing Co., Easton, Pa.

A variety of administration routes are available. The particular mode selected will depend of course, upon the particular composition selected, the severity of the condition being treated and the dosage required for therapeutic efficacy. The methods of the invention, generally speaking, may be practiced using any mode of administration that is medically acceptable, meaning any mode that produces effective levels of the active compounds without causing clinically unacceptable adverse effects. Such modes of administration include oral, rectal, topical, nasal, interdermal, or parenteral routes. The term “parenterall”i ncludes subcutaneous, intravenous, intramuscular, or infusion. Intravenous or intramuscular routes are not particularly suitable for long-term therapy and prophylaxis. They could, however, be preferred in emergency situati polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono-di-and tri-glycerides; hydrogel release systems; sylastic systems; peptide based systems; wax coatings; compressed tablets using conventional binders and excipients; partially fused implants; and the like. Specific examples include, but are not limited to: (a) erosional systems in which the compositions of the invention is contained in a form within a matrix and (b) difusional systems in which an active component permeates at a controlled rate from a polymer. In addition, pump-based hardware delivery systems can be used, some of which are adapted for implantation.

Use of a long-term sustained release implant may be particularly suitable for treatment of chronic conditions. Long-term release, are used herein, means that the implant is constructed and arranged to delivery therapeutic levels of the active ingredient for at least 30 days, and preferably 60 days. Long-term sustained release implants are well-known to those of ordinary skill in the art and include some of the release systems described above.

According to another aspect the present invention relates to the use of immunostimulatory dI/dU oligonucleotides in the prevention and treatment of parasitic infection and disease.

Parasites are organisms which depend upon other organisms in order to survive and thus must enter, or infect, another organism to continue their life cycle. The infected organism, i. e., the host, provides both nutrition and habitat to the parasite. Although in its broadest sense the term parasite can include all infectious agents (i. e., bacteria, viruses, fungi, protozoa and helminths), generally speaking, the term herein is used to refer solely to protozoa, helminths, and ectoparasitic arthropods (e. g., ticks, mites, etc.). Protozoa are single celled organisms which can replicate both intracellularly and extracellularly, particularly in the blood, intestinal tract or the extracellular matrix of tissues. Helminths are multicellular organisms which almost always are extracellular (the exception being Trichinella spp.).

Helminths normally require exit from a primary host and transmission into a secondary host in order to replicate. In contrast to these aforementioned classes, ectoparasitic arthropods form a parasitic relationship with the external surface of the host body.

Rarely is the parasite-host relationship symbiotic, with both the parasite and the host benefiting from the interaction. Instead, parasitic infections, particularly helmintic infections, and the diseases to which they give rise, are chronic conditions, due to the initial asymptomatic presence of some parasites. In extreme instances the infection, and the related disease, are acute and, if left untreated, can be lethal. These latter instances represent a small proportion of total parasitic infections, most probably because the parasite is ultimately dependent upon a viable host in order to propagate.

Parasites are capable of infecting almost any tissue or cell type, however, depending on the particular parasite, they tend to preferentially target a subset of cells including, in humans, red cells, fibroblasts, muscle cells, macrophages and hepatocytes. For example, the protozoan Entamoeba histolytica which is found in the intestinal tract and propagated by contact with host feces, can migrate across the intestinal mucosal lining to infect other bodily tissues such as the liver eventually forming amoebic abscesses. Other parasites can be transmitted via intermediate hosts such as mosquitoes. Ectoparasitic arthropods are a nuisance for household pets (e. g., dogs, cats) and, more importantly, can contribute to wasting syndromes and act as a vehicle for the transmission of other infections (such as babesiosis and theileriasis) in agricultural livestock.

Malaria is the most prevalent parasitic disease in humans. It is estimated that malariacausing parasites such as Plasmodium falciparum, Plasmodium vivax, Plasmodium malariae, Plasmodium knowlesi and Plasmodium ovale result in an estimated 300-500 million new infections and 1.5 to 2.7 million deaths annually in less developed areas of the world (WHO, 1995). In addition, tens of millions of travelers from countries, where malaria is not endemic, visit countries where it is, and many of these travelers succumb to illness during their travels or after returning home. In the latter case, there is a particular risk of failure to rapidly diagnose and initiate treatment, owing to the lack of experience with the disease by local physicians.

Other parasitic infections in humans include schistosomiasis, filariasis, hookworm, ascariasis, leishmaniasis, trichinosis, Chagas'disease and African trypanosomiasis.

In addition to the human health risks, parasites also pose a considerable risk to agricultural livestock and domestic and wild animals. Agricultural livestock and in some cases zoo animals are ripe targets for widespread transmission of parasitic diseases for two major reasons. First, livestock usually live in such close quarters thereby facilitating the transmission of a parasite to an entire flock or herd. Second, because many enteric parasites eventually exit the body in feces which invariably litter a grazing field for animals, the likelihood of transmission and widespread infection is high. Thus the maintenance of a parasite free environment through prevention of parasitic infections would be highly desirable in these circumstances.

The elimination of parasites by the immune system is usually incomplete due in part to the complex and varied life cycles of parasites which consist of antigenically distinct developmental stages. The immune response to parasitic invasion is generally not humoral (i. e., antibody based) and thus immunological memory does not usually follow from an infection. As a result, infected individuals do not develop an immunity to the parasite and continue to be susceptible to future infections.

The treatment and prevention of parasitic infection has traditionally depended on the discovery of drugs targeted against the particular parasite or a carrier of the parasite, such as mosquitoes (e. g., insecticides). Although historically productive, many of the parasites, particularly those that cause malaria, have now developed resistance to such drugs and there are few new drug candidates on the horizon. Thus new and more effective methods to prevent and treat this widespread and serious disease are required. Considerable effort has been put into the development of vaccines designed to induce specific anti-parasite immune responses. While there has been substantial progress in this endeavor, no anti-malarial vaccine has ever been licensed.

The present invention therefore also relates to the use of dI/dU, especially CpdI/dU, oligonucleotides in the prevention and treatment of parasitic infections and related diseases.

In one aspect, the invention relates to a method for preventing a parasitic infection in a subject comprising administering to the subject at risk of being infected with a parasite an effective amount, for preventing a parasitic infection, of a dI7Du containing ODN, especially an oligonucleotide having a sequence including at least the following formula: 5′X1 C(dI/dU)X2 3 wherein the oligonucleotide includes at least 6 nucleotides wherein C and dI/dU are unmethylated and wherein Xland X2 are nucleotides prior to exposure to a parasite.

In some embodiments of the invention, the subject at risk of being infected with a parasite is a human. In still other embodiments the subject is non-human. In still further embodiments, the invention is directed towards a subject selected from the group consisting of a cat, dog, cow, pig, sheep, horse, chicken, duck, goose, fish, goat, mouse, rat, gerbil, rabbit and a zoo animal.

In one embodiment of the invention, the subject is at risk of infection with an intracellular parasite. In another embodiment, the parasite is an obligate intracellular parasite.

In still a further embodiment, the method of the invention is directed towards the prevention of infection by the following parasites: Plasmodium falciparum, Plasmodium ovale, Plasmodium malariae, Plasmdodium vivax, Plasmodium knowlesi, Babesia microti, Babesia divergens, Trypanosoma cruzi, Toxoplasma gondii, Trichinella spiralis, Leishmania major, Leishmania donovani, Leishmania braziliensis and Leishmania tropica. In another embodiment, the method is directed towards the prevention of infection by the following parasites: Trypanosoma gambiense, Trypanosmoma rhodesiense and Schistosoma mansoni.

In preferred embodiments, the method is directed towards the prevention of infection with parasites which cause malaria.

In one embodiment of the invention, the subject is also administered an effective amount of one or more pdI/dU oligonucleotide therapeutic agents. In preferred embodiments, the dI/dU oligonucleotide therapeutic agent is a parasiticide. In other preferred embodiments, the dI/dU oligonucleotide therapeutic agent is selected from the group consisting of IL-1, IL-6, IL-12, IL-15, IL-18, IFN-y, TNF-a, GM-CSF, CD40 ligand and Flt3 ligand. In some embodiments in which IL-12 and IFN-y are administered, IL-12 is administered prior to IFN administration.

In one embodiment of the invention, the oligonucleotide is administered more than once. In other embodiments, the oligonucleotide is administered at least 7 days prior to a parasite infection. In still other embodiments, the oligonucleotide is administered at least 2 days prior to a parasite infection. In still further embodiments, the oligonucleotide is administered at least 24 hours prior to a parasite infection.

In one aspect, the invention involves a method for preventing parasitic infection in a subject. Parasitic infection arises from exposure to parasites which can occur in a number of ways. Transmission is possible through contact with bodily fluids, tissues or waste products from infected individuals, or through contact with intermediary hosts such as insects (e. g., insect bites). Individuals who are infected with parasites can be identified based on physical symptoms and/or clinical findings including the observation of parasitic bodies or debris in samples of bodily fluids, tissues or waste.

In one aspect, the methods of the invention involve administering to a subject, at risk of being infected with a parasite, a dI/dU containing oligonucleotide in an amount effective to prevent a parasitic infection in the subject. As defined herein, an individual “at risk of being infected with a parasite” is one who has any risk of exposure to an infectious parasite such as conditions or environments in which parasite infections are common, including contact with an infected individual. A subject is at risk of parasitic infection if there is a possibility that the subject will be exposed or come in contact with another individual either known to be or later diagnosed as suffering from a parasitic infection. For example, an individual anticipating travel to a region in which parasitic infections are endemic is considered a person at risk of being infected with a parasite. The prevalence in some countries of parasites, and the diseases to which they give rise, increases the likelihood that travelers, workers and military personnel assigned to these regions will be at risk of parasitic exposure and subsequently, suffer from a parasitic infection.

In addition to the use of the dI/dU oligonucleotide for prophylactic treatment, the invention also encompasses the use of the dI/dU oligonucleotide for the treatment of a subject having a parasite infection. A “subject having a parasite infection” is a subject that has been exposed to an infectious parasite and has acute or chronic detectable levels of the pathogen in the body. The dI/dU oligonucleotide can be used to mount an innate immune response that is capable of reducing the level of or eradicating the infectious pathogen (i. e., parasite). The innate immune response does not involve an antigen and is thus useful against any type of pathogen. In addition to the innate immune response the dI/dU oligonucleotide may also enhance an antigen specific immune response if an antigen is administered with the dI/dU oligonucleotide. An antigen specific immune response, however, is not required for prophylactic or treatment purposes according to the invention. An infectious parasitic disease, as used herein, is a disease arising from the presence of a parasite in the body.

In preferred embodiments, the subject has been exposed to malaria causing Plasmodium spp. In other embodiments, the subject has been infected with Trypanosoma cruzi, Trichinella spiralis, Babesia spp. or Toxoplasma gondii. When used as a mode of treatment, the dI/dU oligonucleotides of the invention can be administered following suspected or confirmed parasite exposure. As will be discussed herein, a subject infected with a parasite often times exhibits a set of symptoms which can be used to identify the presence of the parasitic infection and in some instances, the particular parasite involved.

Parasitic infections which the compounds and methods of the invention seek to prevent and treat include those occurring in humans and non-human vertebrates. According to some embodiments, the methods of the invention are directed towards human subjects. In yet other embodiments, the methods of the invention are directed towards non-human vertebrates including agricultural live-stock and domesticated and wild animals, such as, for example, cattle, horses, swine, goats and sheep, poultry and other winged vertebrates, rabbits, dogs, cats, ferrets and fish. Non-human vertebrates which exist in close quarters and which are allowed to intermingle as in the case of zoo and research animals are also embraced as subjects for the methods of the invention. Zoo animals such as the felid species including for example lions, tigers, leopards, cheetahs, and cougars; elephants, giraffes, bears, deer, wolves, yaks, non-human primates, seals. dolphins and whales; and research animals such as mice, rats, hamsters and gerbils are all potential subjects for the methods of the invention.

The methods of the invention when used prophylactically embrace the prevention of infection from parasitic species to which the vertebrate subjects are vulnerable. Most parasites are host-specific or have a limited host range, i. e., they are able to infect a single or at most a few species. For example, P. yoelii is able to infect only rodents while P. falciparum and P. malariae are able to infect humans. The parasitic infection to be targeted by the methods and compounds of the invention will depend upon the host species receiving the prophylactic treatment and the conditions to which that host will become exposed.

Parasites can be classified based on whether they are intracellular or extracellular. An “intracellular parasite” as used herein is a parasite whose entire life cycle is intracellular.

Examples of human intracellular parasites include Leishmania spp., Plasmodium spp., Trypanosoma cruzi, Toxoplasma gondii, Babesia spp., and Trichinella spiralis. An “extracellular parasite” as used herein is a parasite whose entire life cycle is extracellular.

Extracellular parasites capable of infecting humans include Entamoeba histolytica, Giardia lamblia, Enterocytozoon bieneusi, Naegleria and Acanthamoeba as well as most helminths.

Yet another class of parasites is defined as being mainly extracellular but with an obligate intracellular existence at a critical stage in their life cycles. Such parasites are referred to herein as “obligate intracellular parasites”. These parasites may exist most of their lives or only a small portion of their lives in an extracellular environment, but they all have at lest one obligate intracellular stage in their life cycles. This latter category of parasites includes Trypanosoma rhodesiense and Trypanosoma gambiense, Isospora spp., Cryptosporidium spp, Eimeria spp., Neospora spp., Sarcocystis spp., and Schistosoma spp. In one aspect, the invention relates to the prevention and treatment of infection resulting from intracellular parasites and obligate intracellular parasites which have at least in one stage of their life cycle that is intracellular. In some embodiments, the invention is directed to the prevention of infection from obligate intracellular parasites which are predominantly intracellular. The methods of the invention are not expected to function in the prevention of infection by extracellular parasites, i. e., helminths. An exemplary and non-limiting list of parasites for some aspects of the invention is provided herein.

Blood-borne and/or tissues parasites include Plasmodium spp., Babesia microti, Babesia divergens, Leishmania tropica, Leishmania spp., Leishmania braziliensis, Leishmania donovani, Trypanosoma gambiense and Trypanosoma rhodesiense (African sleeping sickness), Trypanosoma cruzi (Chagas'disease), and Toxoplasma gondii.

Typical parasites infecting horses are Gasterophilus spp.; Eimeria leuckarti, Giardia spp.; Tritrichomonas equi; Babesia spp. (RBC's), Theileria equi; Trypanosoma spp.; Klossiella equi; Sarcocystis spp.

Typical parasites infecting swine include Eimeria bebliecki, Eimeria scabra, Isospora suis, Giardia spp.; Balantidium coli, Entamoeba histolytica; Toxoplasma gondii and Sarcocystis spp., and Trichinella spiralis.

The major parasites of dairy and beef cattle include Eimeria spp., Cryptosporidium sp., Giardia sp.; Toxoplasma gondii; Babesia bovis (RBC), Babesia bigemina (RBC), Trypanosoma spp. (plasma), Theileria spp. (RBC); Theileria parva (lymphocytes); Tritrichomonas foetus; and Sarcocystis spp.

The major parasites of raptors include Trichomonas gallinae; Coccidia (Eimeria spp.); Plasmodium relictum, Leucocytozoon danilewskyi (owls), Haemoproteus spp., Trypanosoma spp.; Histomonas; Cryptosporidium meleagridis, Cryptosporidium baileyi, Giardia, Eimeria; Toxoplasma.

Typical parasites infecting sheep and goats include Eimeria spp., Cryptosporidium sp., Giardia sp.; Toxoplasma gondii ; Babesia spp. (RBC), Trypanosoma spp. (plasma), Theileria spp. (RBC); and Sarcocystis spp.

Typical parasitic infections in poultry include coccidiosis caused by Eimeria acervulina, E. necatrix, E. tenella, Isospora spp. and Eimeria truncata; histomoniasis, caused by Histomonas meleagridis and Histomonas gallinarum; trichomoniasis caused by Trichomonas gallinae; and hexamitiasis caused by Hexamita meleagridis. Poultry can also be infected Emeria maxima, Emeria meleagridis, Bimeria adenoeides, Eimeria meleagrimitis, Cryptosporidium, Eimeria brunetti, Emeria adenoeides, Leucocytozoon spp., Plasmodium spp., Hemoproteus meleagridis, Toxoplasma gondii and Sarcocystis.

Parasitic infections also pose serious problems in laboratory research settings involving animal colonies. Some examples of laboratory animals intended to be treated, or in which parasite infection is sought to be prevented, by the methods of the invention include mice, rats, rabbits, guinea pigs, nonhuman primates, as well as the aforementioned swine and sheep.

Typical parasites in mice include Leishmania spp., Plasmodium berghei, Plasmodium yoelii, Giardia muris, Hexamita muris; Toxoplasma gondii; Trypanosoma duttoni (plasma); Klossiella muris; Sarcocystis spp. Typical parasites in rats include Giardia muris, Hexamita muris; Toxoplasma gondii; Trypanosoma lewisi (plasma); Trichinella spiralis; Sarcocystis spp. Typical parasites in rabbits include Eimeria sp.; Toxoplasma gondii; Nosema cuniculi; Eimeria stiedae, Sarcocystis spp. Typical parasites of the hamster include Trichomonas spp.; Toxoplasma gondii; Trichinella spiralis; Sarcocystis spp. Typical parasites in the guinea pig include Balantidium caviae; Toxoplasma gondii; Klossiella caviae; Sarcocystis spp.

The methods of the invention can also be applied to the treatment and/or prevention of parasitic infection in dogs, cats, birds, fish and ferrets. Typical parasites of birds include Trichomonas gallinae; Eimeria spp., Isospora spp., Giardia; Cryptosporidium; Sarcocystis spp., Toxoplasma gondii, Haemoproteus/Parahaemoproteus, Plasmodium spp., Leucocytozoon-lAkiba, Atoxoplasma, Trypanosoma spp. Typical parasites infecting dogs include Trichinella spiralis; Isopora spp., Sarcocystis spp., Cryptosporidium spp., Hammondia spp., Giardia duodenalis (canis); Balantidium coli, Entamoeba histolytica; Hepatozoon canis; Toxoplasma gondii, Trypanosoma cruzi; Babesia canis; Leishmania amastigotes; Neospora caninum.

Typical parasites infecting feline species include Isospora spp., Toxoplasma gondii, Sarcocystis spp., Hammondia hammondi, Besnoitia spp., Giardia spp.; Entamoeba histolytica; Hepatozoon canis, Cytauxzoon sp., Cytauxzoon sp., Cytauxzoon sp. (red cells, RE cells).

Typical parasites infecting fish include Hexamita spp., Eimeria spp.; Cryptobia spp., Nosema spp., Myxosoma spp., Chilodonella spp., Trichodina spp. ; Plistophora spp., Myxosoma Henneguya; Costia spp., Ichthyophithirius spp., and Oodinium spp.

Typical parasites of wild mammals include Giardia spp. (carnivores, herbivores), Isospora spp. (carnivores), Eimeria spp. (carnivores, herbivores); Theileria spp. (herbivores), Babesia spp. (carnivores, herbivores), Trypanosoma spp. (carnivores, herbivores); Schistosoma spp. (herbivores); Fasciola hepatica (herbivores), Fascioloides magna (herbivores), Fasciola gigantica (herbivores), Trichinella spiralis (carnivores, herbivores).

Parasitic infections in zoos can also pose serious problems. Typical parasites of the bovidae family (blesbok, antelope, banteng, eland, gaur, impala, klipspringer, kudu, gazelle) include Eimeria spp. Typical parasites in the pinnipedae family (seal, sea lion) include Eimeria phocae. Typical parasites in the camelidae family (camels, llamas) include Eimeria spp. Typical parasites of the giraffidae family (giraffes) include Eimeria spp. Typical parasites in the elephantidae family (African and Asian) include Fasciola spp. Typical parasites of lower primates (chimpanzees, orangutans, apes, baboons, macaques, monkeys) include Giardia sp.; Balantidium coli, Entamoeba histolytica, Sarcocystis spp., Toxoplasma gondii; Plasmodim spp. (RBC), Babesia spp. (RBC), Trypanosoma spp. (plasma), Leishmania spp. (macrophages).

Diseases caused by parasites can be acute, as in the case of malaria (Plasmodium falciparum, P. vivax, P. ovale, P. malariae) or AIDS-related opportunistic pathogenic infection (Toxoplasma and Cryptosporidium), or chronic, as with heart disease in South America (Trypanosoma cruzi), fluke-like disease (schistosomiasis) and blindness (Onchocerca volvulus) in humans. Parasite-related diseases also include: in cattle, ostertagiasis caused by Ostertagia infection and manifest as diarrhea, anorexia or loss of appetite and weight loss; in sheep, haemonchosis caused by H. contortus infection and manifest as unexpected death, weakness, anemia, hypoproteinemia, subcutaneous edema, weight loss, or poor or no weight gain.

According to some aspects of the invention, the subject is free of parasitic infection and disease related symptoms. In some instances, subjects have b malaise, lethargy, fatigue, headache, fever, chills, weakness, fast heartbeat, heart pain. blurry or unclear vision, anemia, loss of appetite, weight loss or failure of weight gain, lower abdominal or back pain ranging from diffuse to severe, diarrhea, numb hands, sexual dysfunction (in male subjects), menstrual irregularity, jaundiced skin colour and itchy orifices including ears, nose and anus. Severe malaria can manifest itself in unarousable coma (cerebral malaria), renal failure, severe anemia, pulmonary edema, hypoglycemia, hypotension or shock, bleeding or disseminated intravascular coagulation, convulsions, acidemia or acidosis, hemoglobinuria, jaundice and hyperpyrexia. Symptoms particularly associated with gastrointestinal parasitic infections also include loss of blood resulting in pale mucous membranes, diarrhea with loss of water and electrolyte disturbances, poor weight gains or even weight loss in severe infections, protein losses, hypoproteinemia and associated edema, anorexia and reduced food intake, anemia, reduced digestion and absorption.

Diagnosis of a parasite infection in non-human animals can involve the initial observance of symptoms associated with particular infections. For example, haemonchosis in sheep should be suspected if the following conditions are observed: unexpected deaths, weakness, anemia, hypoproteinemia, subcutaneous edema, poor weight gains or weight loss.

These conditions will be apparent and well known to a veterinarian.

The diagnosis of a parasitic infection in an individual can be used to determine the need for prophylactic treatment in other subjects previously in contact or likely to be in contact with the afflicted individual using the methods of the invention as well as for treatment of the infected individual. A number of laboratory tests for the diagnosis of parasitic infections, well known in the art, are described.

Methods for diagnosing parasitic infections are generally similar for human and nonhuman parasitic infections. Procedures for diagnosing parasitism vary depending on the type of parasite to be detected. These procedures are well known to any clinician or veterinarian and can be easily performed in almost any clinical or veterinary practice. Macroscopic and microscopic examination of a bodily sample is usually initially performed to detect the presence of ova and adult parasites. Tissue parasites can sometimes be detected through the examination of biopsies and aspirates. A bodily sample can be a liquid such as urine, saliva, cerebrospinal fluid, blood, serum, bronchoalveolar lavage, sputum, bile or the like; a solid or semi-solid such as tissues, feces, or the like; or, alternatively, a solid tissue such as those commonly used in histological diagnosis.

Tests for parasites in agricultural livestock include direct smear of bodily liquid such as blood or bodily waste such as feces; fecal flotation fluids, centrifugation technique with flotation fluid (magnesium sulfate), modified Wisconsin Procedure for egg counts by a flotation method (for cattle, horses, dogs, cats and swine), modified Knott's Method of concentrating microfilaria, skin scraping and squash preparation for the diagnosis of trichinosis. Generally liquid samples should be stained in order to better visualize any parasite bodies. Giemsa or Wright's stain are appropriate for analysis of a number of parasites including Plasmodium spp., Leishmania spp., African trypanosomes, Trypanosoma cruzi, Toxoplasma gondii and Naegleria fowleri in the blood, urine or spinal fluid.

A diagnosis of coccidiosis in poultry can be established by preparing a wet mount of a mucosal scraping from the intestines of an infected bird and examining it by light microscopy.

The coccidial oocytes and schizonts can readily be identified at 100× magnification. A fecal flotation is also very effective in demonstrating coccidial oocysts. Histomoniasis can be diagnosed based on characteristic gross lesions and/or histologic lesions and H. gallinarum can be isolated from tissues of freshly killed affected birds in special broth media.

In one aspect, the methods of the invention involve administering to a subject, prior to parasite exposure, a dI/dU containing oligonucleotide in an amount effective to prevent a parasitic infection in the subject. Prior administration of a dI/dU oligonucleotide greatly benefits the subject by inducing a response within the subject consisting at least of an activated innate immune system response prior to, during or following the exposure to a parasite. By “prior administration” it is meant that administration occurs before exposure to the parasite. In some embodiments, the compounds of the invention may be administered with a greater than 60 day period of time between the administration and the parasite exposure. In other embodiments, the dI/dU oligonucleotides may be administered at least 50, or 40, or 30, or 14, or 7 days prior to parasite exposure. In yet other embodiments, the dI/dU oligonucleotides may be administered within a 7, 6, 5, 4, 3 or 2 day period prior to infection.

In still other embodiments, the dI/dU oligonucleotide of the invention may be administered at least 24 hours prior to suspected parasitic exposure. And in still further embodiments, the dI/dU oligonucleotides may be administered within 24,12 or 4 hours of parasite infection.

Timing will depend upon the particular parasite infection to be treated and/or prevented as well as the mode of delivery (i. e., whether acute or chronic release required). If chronic delivery or treatment is required, then, in some embodiments, dI/dU oligonucleotides may be administered with a greater than 7 day period between the dI/dU oligonucleotide administration and the parasite exposure. In such cases, higher doses may be used but are not always required. In preferred embodiments, the dI/dU oligonucleotides are administered within 2 days of parasite exposure. The period of protection will depend upon the dose of dI/dU oligonucleotide administered, thus high doses can provide longer lasting protection. The length of protection will also depend upon the mode of administration and the particular infection being prevented. Administration may also be repeated, such that a more prolonged anti-parasitic effect can be obtained following multiple treatments with dI/dU oligonucleotides or delivery of dI/dU oligonucleotides in controlled release vesicles (e. g., micro encapsulated) or formulated in such a way to retard in vivo degradation (e. g., liposomes).

In another aspect, the invention relates to the treatment of subjects infected with a parasite. In preferred embodiments the subject has been exposed and is currently suffering from an infection by the following parasites: Plasmodium spp., Babesia spp., Trypanosoma cruzi, Toxoplasma gondii and Trichinella spiralis. In these embodiments, the dI/dU oligonucleotides are effective in treating the infection even if administered after exposure to the parasite. The compounds of the invention may be administered immediately after the parasite exposure or after a period of time. For example, the dI/dU oligonucleotides may be administered once the parasitic infection has been diagnosed which may range from a few days to several weeks after parasite exposure or contact. In some embodiments, the dI/dU oligonucleotides may be administered within 24 hours or 48 hours after parasite infection (i. e., parasite exposure). If diagnosis or treatment is delayed, it is also envisioned that the oligonucleotides may be administered within 7 days of infection. There may still be other situations in which even longer (i. e., greater than 7 days, 14 days or 30 days) period of time may elapse between parasite exposure and oligonucleotide administration.

Coadministered compounds may be those known to be active against a particular parasitic disease. Examples of parasiticides useful for human administration include but are not limited to albendazole, amphotericin B, benznidazole, bithionol. chloroquine HCI, chloroquine phosphate, clindamycin, dehydroemetine, diethylcarbamazine, diloxanide furoate, eflornithine, furazolidaone, glucocorticoids, halofantrine, iodoquinol, ivermectin, mebendazole, mefloquine, meglumine antimoniate, melarsoprol, metrifonate, metronidazole, niclosamide, nifurtimox, oxamniquine, paromomycin, pentamidine isethionate, piperazine, praziquantel, primaquine phosphate, proguanil, pyrantel pamoate, pyrimethanminesulfonamides, pyrimethanmine-sulfadoxine, quinacrine HCI, quinine sulfate, quinidine gluconate, spiramycin, stibogluconate sodium (sodium antimony gluconate), suramin, tetracycline, doxycycline, thiabendazole, tinidazole, trimethroprim-sulfamethoxazole, and tryparsamide some of which are used alone or in combination with others.

Parasiticides used in non-human subjects include piperazine, diethylcarbamazine, thiabendazole, fenbendazole, albendazole, oxfendazole, oxibendazole, febantel, levamisole, pyrantel tartrate, pyrantel pamoate, dichlorvos, ivermectin, doramectic, milbemycin oxime, iprinomectin, moxidectin, N-butyl chloride, toluene, hygromycin B thiacetarsemide sodium, melarsomine, praziquantel, epsiprantel, benzimidazoles such as fenbendazole, albendazole, oxfendazole, clorsulon, albendazole, amprolium; decoquinate, lasalocid, monensin sulfadimethoxine; sulfamethazine, sulfaquinoxaline, metronidazole.

Parasiticides used in horses include mebendazole, oxfendazole, febantel, pyrantel, dichlorvos, trichlorfon, ivermectin, piperazine; for S. westeri: ivermectin, benzimiddazoles such as thiabendazole, cambendazole, oxibendazole and fenbendazole. Useful parasiticides in dogs include milbemycin oxine, ivermectin, pyrantel pamoate and the combination of ivermectin and pyrantel. The treatment of parasites in swine can include the use of levamisole, piperazine, pyrantel, thiabendazole, dichlorvos and fenbendazole. In sheep and goats anthelmintic agents include levamisole or ivermectin. Caparsolate has shown some efficacy in the treatment of D. immitis (heartworm) in cats.

Agents used in the prevention and treatment of protozoal diseases in poultry, particularly trichomoniasis, can be administered in the feed or in the drinking water and include protozoacides such as aminonitrothiazole, dimetridazole (Emtryl), nithiazide (Hepzide) and Enheptin. However, some of these drugs are no longer available for use in agrigultural stocks in the USA. Back yard flocks or pigeons not used for food production may be effectively treated with dimetridazole by prescription of a veterinarian (1000 mg/L in drinking water for 5-7 days).

The present invention further relates to the use of the dI/dU oligonucleotides according to the present invention for the activation of human PBMC, human myeloid dendritic cells and human plasmacytoid cells. These cells may be specifically and strongly induced by the molecules according to the present invention. This induction is especially preferred when immune responses of a certain kind are necessary, e.g. if naive T-cells are necessary to be induced.

The dI/dU oligonucleotide of the present invention preferably has a sequence including at least the following formula: 5′ X1 C(dI/dU)X2 3′. In one preferred embodiment the invention provides a CpdI/dU oligonucleotide represented by at least the formula: 5′N, X1 C(dI/dU)X2N2 3′ wherein at least one nucleotide separates consecutive CpdI/dUs; X, is adenine, deoxyinosine/deoxyuridine, or thymine; X is cytosine, adenine, or thymine; N is any nucleotide and N, and N, are nucleic acid sequences composed of from about 0-25 N's each.

In another embodiment the invention provides an isolated CpdI/dU oligonucleotide represented by at least the formula: 5′N1X1X2C(dI/dU)X3X4N23′ wherein at least one nucleotide separates consecutive CpdI/dUs; X1 X2 is selected from the group consisting of GpT, GpG, GpA, ApA, ApT, ApG, CpT, CpA, CpdI/dU, TpA, TpT, and TpG; X3X4 is selected from the group consisting of TpT, CpT, ApT, TpG, ApG, CpdI/dU, TpC, ApC, CpC, TpA, ApA, and CpA; N is any nucleotide and N1 and N2 are nucleic acid sequences composed of from about 0-25 N's each. In a preferred embodiment N1 and N2 of the nucleic acid do not contain a CC(dI/dU)G or a C(dI/dU)C(dI/dU) quadmer or more than one CC(dI/dU) or C(dI/dU)G trimer especially if the oligonucleotide has a modified phosphate backbone.

Preferably, the ODN according to the present invention contains at least one structure represented by the following general formula:
5′-NMPn . . . NMP3NMP2NMP1NMP1′NMP2′NMP3′. . . NMPn′-3′ (II)
(wherein n is an integer from 3 to 50; NMPl, NMP2, NMP3, . . . , NMPn and NMP1′, NMP2′, NMP3′, . . . , NMPn′ are each a monodeoxyribonucleotide; NMP1, NMP2, NMP3, . . . and Xn may be the same or different nucleotides, wherein at least one of said monodeoxyribonucleotides is dI or dU; and bases in NMP1 and NMP1′, in NMP2 and NMP2′, in NMP3 and NMP3′, in . . . , and in NMPn and NMPn′ are, except dI or dU residues, complementary with each other as defined by Watson & Crick) or a salt thereof.

According to a further aspect, the present invention also relates to the use of the ODNs according to the present invention for the preparation of a medicine for activating a subject's antigen presenting cells.

Preferably the pharmaceutical composition according to the present invention further comprises a polycationic polymer, preferably a polycationic peptide, especially polyarginine, polylysine or an antimicrobial peptide.

The polycationic compound(s) to be used according to the present invention may be any polycationic compound which shows the characteristic effect according to the WO 97/30721. Preferred polycationic compounds are selected from basic polypeptides, organic polycations, basic polyaminoacids or mixtures thereof. These polyaminoacids should have a chain length of at least 4 amino acid residues. Especially preferred are substances containing peptidic bounds, like polylysine, polyarginine and polypeptides containing more than 20%, especially more than 50% of basic amino acids in a range of more than 8, especially more than 20, amino acid residues or mixtures thereof. Other preferred polycations and their pharmaceutical compositions are described in WO 97/30721 (e.g. polyethyleneimine) and WO 99/38528. Preferably these polypeptides contain between 20 and 500 amino acid residues, especially between 30 and 200 residues.

These polycationic compounds may be produced chemically or recombinantly or may be derived from natural sources.

Cationic (poly)peptides may also be polycationic anti-bacterial microbial peptides. These (poly)peptides may be of prokaryotic or animal or plant origin or may be produced chemically or recombinantly. Peptides may also belong to the class of defensins. Such host defense peptides or defensives are also a preferred form of the polycationic polymer according to the present invention. Generally, a compound allowing as an end product activation (or down-regulation) of the adaptive immune system, preferably mediated by APCs (including dendritic cells) is used as polycationic polymer.

Especially preferred for use as polycationic substance in the present invention are cathelicidin derived antimicrobial peptides or derivatives thereof (A 1416/2000, incorporated herein by reference), especially antimicrobial peptides derived from mammal cathelicidin, preferably from human, bovine or mouse, or neuroactive compounds, such as (human) growth hormone (as described e.g. in WO01/24822).

Polycationic compounds derived from natural sources include HIV-REV or HIV-TAT (derived cationic peptides, antennapedia peptides, chitosan or other derivatives of chitin) or other peptides derived from these peptides or proteins by biochemical or recombinant production. Other preferred polycationic compounds are cathelin or related or derived substances from cathelin, especially mouse, bovine or especially human cathelins and/or cathelicidins. Related or derived cathelin substances contain the whole or parts of the cathelin sequence with at least 15-20 amino acid residues. Derivations may include the substitution or modification of the natural amino acids by amino acids which are not among the 20 standard amino acids. Moreover, further cationic residues may be introduced into such cathelin molecules. These cathelin molecules are preferred to be combined with the antigen/vaccine composition according to the present invention. However, these cathelin molecules surprisingly have turned out to be also effective as an adjuvant for a antigen without the addition of further adjuvants. It is therefore possible to use such cathelin molecules as efficient adjuvants in the present medicines with or without further immunactivating substances.

Another preferred polycationic substance to be used according to the present invention is a synthetic peptide containing at least 2 KLK-motifs separated by a linker of 3 to 7 hydrophobic amino acids, especially L (A 1789/2-000, incorporated herein by reference).

In one particular embodiment, the preferred vehicle for the ODN according to the present invention is a biocompatible microparticle or implant that is suitable for implantation into a vertebrate recipient. In accordance with the instant invention, the dI/dU containing oligonucleotides described herein are encapsulated or dispersed within the biocompatible, preferably biodegradable polymeric matrix. The polymeric matrix preferably is in the form of a microparticle such as a microsphere (wherein the dI/dU oligonucleotide is dispersed throughout a solid polymeric matrix) or a microcapsule (wherein the dI/dU oligonucleotide is stored in the core of a polymeric shell). Other forms of the polymeric matrix for containing the dI/dU oligonucleotide include films, coatings, gels, implants, and stents. The size and composition of the polymeric matrix device can be selected to result in favorable release kinetics in the tissue into which the matrix device is implanted. Alternatively, the implant may be designed such that it releases sufficient levels of the dI/dU oligonucleotide to provide systemic exposure.

The size of the polymeric matrix devise can be further selected according to the method of delivery which is to be used, typically injection into a tissue or administration of a suspension by aerosol into the nasal and/or pulmonary areas. The polymeric matrix composition can be selected to have both favorable degradation rates and also to be formed of a material which is bioadhesive, to further increase the effectiveness of transfer when the devise is administered to a particular surface or tissue. The matrix composition also can be selected not to degrade, but rather, to release by diffusion over an extended period of time.

Both non-biodegradable and biodegradable polymeric matrices can be used to deliver the dI/dU oligonucleotide of the invention to the subject. Such polymers may be natural or synthetic polymers. Synthetic polymers are preferred. The polymer is selected based on the period of time over which release is desired, generally in the order of a few hours to a year or longer. The period of sustained release will depend upon the subject and the environment.

Typically, release over a period ranging from between a few hours and three to twelve months is most desirable. The polymer optionally is in the form of a hydrogel that can absorb up to about 90% of its weight in water and further, optionally is cross-linked with multi-valent ions or other polymers.

In general, the dI/dU oligonucleotides of the invention may be delivered using the bioerodible implant by way of diffusion, or more preferably, by degradation of the polymeric matrix. Exemplary synthetic polymers which can be used to form the biodegradable delivery system include: polyamides, polycarbonates, polyalkylenes, polyalkylene glycols, polyalkylene oxides, polyalkylene terepthalates, polyvinyl alcohols, polyvinyl ethers, polyvinyl esters, poly-vinyl halides, polyvinylpyrrolidone, polyglycolides, polysiloxanes, polyurethanes and co-polymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly (methyl methacrylate), poly (ethyl methacrylate), poly (butylmethacrylate), poly (isobutyl methacrylate), poly (hexylmethacrylate), poly (isodecyl methacrylate), poly (lauryl methacrylate), poly (phenyl methacrylate), poly (methyl acrylate), poly (isopropyl acrylate), poly (isobutyl acrylate), poly (octadecyl acrylate), polyethylene, polypropylene, poly (ethylene glycol), poly (ethylene oxide), poly (ethylene terephthalate), poly (vinyl alcohols), polyvinyl acetate, poly vinyl chloride, polystyrene and polyvinylpyrrolidone.

Examples of non-biodegradable polymers include ethylene vinyl acetate, poly (meth) acrylic acid, polyamides, copolymers and mixtures thereof.

Examples of biodegradable polymers include synthetic polymers such as polymers of lactic acid and glycolic acid, polyanhydrides, poly (ortho) esters, polyurethanes, poly (butic acid), poly (valeric acid), and poly (lactide-cocaprolactone), and natural polymers such as alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion.

Bioadhesive polymers useful in the invention include bioerodible hydrogels, polyhyaluronic acids, casein, gelatin, glutin, polyanhydrides, polyacrylic acid, alginate, chitosan, poly (methyl methacrylates), poly (ethyl methacrylates), poly (butylmethacrylate), poly (isobutyl methacrylate), poly (hexylmethacrylate), poly (isodecyl methacrylate), poly (lauryl methacrylate), poly (phenyl methacrylate), poly (methyl acrylate), poly (isopropyl acrylate), poly (isobutyl acrylate), and poly (octadecyl acrylate). Thus, the invention provides a composition of the above-described CpdI/dU oligonucleotide for use as a medicament, methods for preparing the medicament and methods for the sustained release of the medicament in vivo.

The materials for use in the invention, either in the administration of the compounds of the invention or in the measure of the bodily levels of these compounds or the factors they induce, are ideally suited for the preparation of a kit. Such a kit may comprise a carrier means being compartmentalized to receive in close confinement one or more container means such as vials, tubes, and the like, each of the container means comprising one of the separate elements to be used in the method. For example, one of the container means may comprise a dI/dU oligonucleotide of the invention. The kit may also have containers comprising other non-dI/dU oligonucleotide therapeutic agents useful in the invention as listed above.

Additionally the kit may include containers for buffer (s) useful in the assay. If the mode of administration is by injection, the kit may also contain an injection delivery device such as an assembled needle and syringe or an autoinjector delivery device, such as those currently in use by the military. Alternatively, the kit may be designed for subcutaneous injection and placement of a long-term sustained release capsule or implant, and would therefore contain an appropriate injection device such as for example a wide-bore needle for transfer of the capsule or implant to a subcutaneous region.

Other kits useful in the invention can comprise means for measuring the extent of the immune response occurring in an individual, thereby indicating whether the individual is sufficiently primed to prevent a parasitic infection. For example, the kit can include means to measure cytokine levels. These kits can be used by the individual or more preferably by a physician, nurse or veterinarian. The kits can be useful in determining whether a longterm release device is continuing to emit the compounds of the invention or in assessing whether a dose modification is necessary. If the kit is meant to measure cytokine or peptide levels in an individual, it will contain a readout system for measuring such a peptide. This readout system may comprise an antibody or other binding peptide which may be prepared on a solid surface such as polystyrene or may be applied to the surface at the time of individual testing. A bodily sample from an individual, preferably a liquid sample such as blood, can then be added either directly or in diluted form onto the surface coated with binding peptide. The binding of components within the sample to the binding peptides of the kit can be measured by the use of a secondary binding peptide conjugated to a label. To be useful, the label should be directly or indirectly detectable or visible. A label which can be visualized using a colorimetric assay is most useful in the invention particularly if no additional instrumentation is required for detection.

Details of the present invention are described by the following examples and the figures, but the invention is of course not limited thereto. It is specifically shown in the examples that the ODNs according to the present invention have comparable or often superior effects compared to e.g. CG motif containing ODNs.

Effects of CG containing ODNs are shown in the examples of EP 0 468 520 A2, WO 96/02555, WO98/18810, WO98/37919, WO98/40100, WO99/51259 and WO099/56755. These examples of the prior art together with the following examples are proving the equivalence or superiority of the present ODNs for the above mentioned uses compared to the CG containing ODNs.

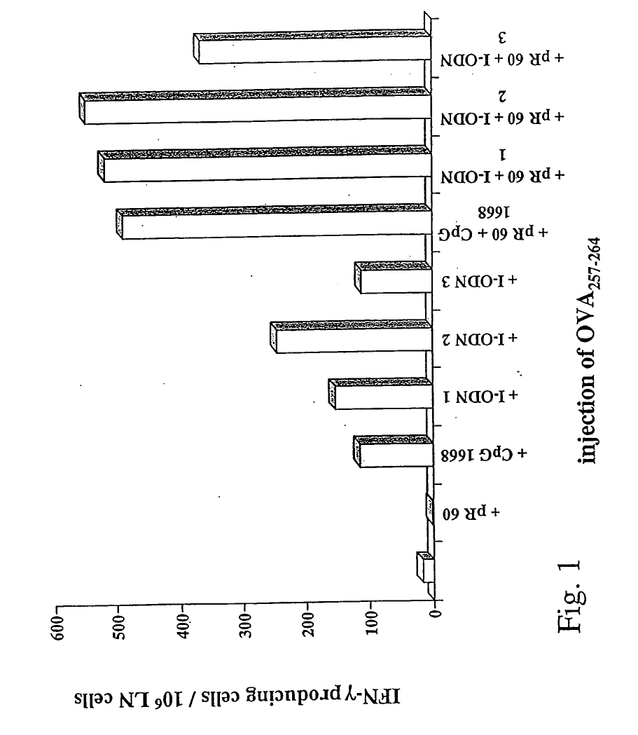

FIG. 1 shows the immune response against the ovalbumin-derived peptide OVA₂₅₇₋₂₆₄ after the injection of OVA₂₅₇₋₂₆₄, poly-L-arginine (pR 60) and deoxyinosine I-containing oligodeoxynucleotides (I-ODN) or CpG 1668. Mice were injected into the hind footpads with mixtures as indicated. Four days later draining lymph node cells were ex vivo stimulated with OVA₂₅₇₋₂₆₄. The number of IFN-g-producing cells was determined 24 hours later using an ELISPOT assay. Results are expressed as the number of spots/1×10⁶ lymph node cells.

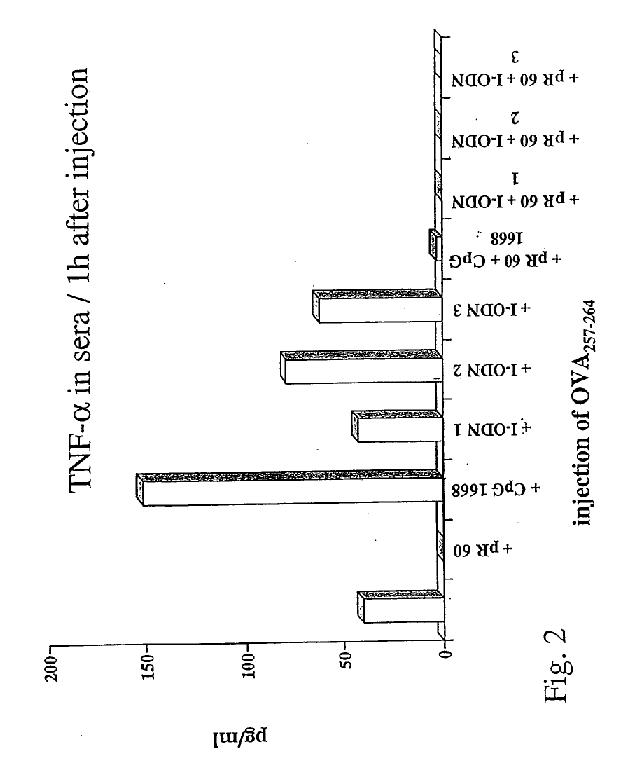

FIG. 2 shows the induction of systemic TNF-a production after the injection of OVA₂₅₇₋₂₆₄, poly-L-arginine (pR 60) and I-containing oligodeoxynucleotides (I-ODN) or CpG 1668. Mice were injected into the hind footpads with mixtures as indicated. One hour after injection blood was taken from the tail vein and serum was prepared. The concentration of TNF-a in the sera was determined using an ELISA.

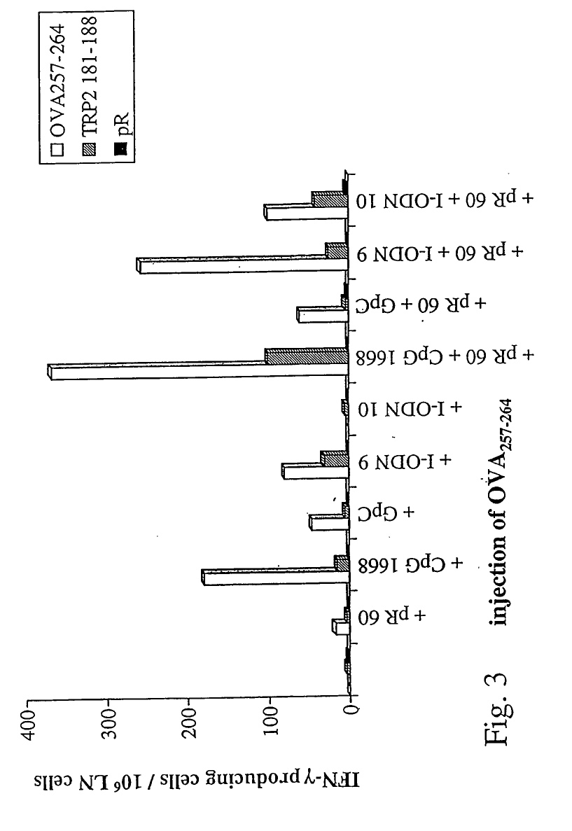

FIG. 3 shows the immune response against the Ovalbumin-derived peptide OVA₂₅₇₋₂₆₄ after the injection of OVA₂₅₇₋₂₆₄, poly-L-arginine (pR60) and deoxyinosine -containing oligodeoxynucleotides (I-ODN), CpG 1668 or GpC. Mice were injected into the hind footpads with mixtures as indicated. Four days later, draining lymph node cells were ex vivo stimulated with OVA₂₅₇₋₂₆₄, an irrelevant peptide mTRP2₁₈₁₋₁₈₈ (murine tyrosinase related protein-2, VYDFFVWL) or pR 60. The number of IFN-g producing cells was determined 24 hours later using an ELISPOT assay. Results are expressed as the number of spots/lxl0⁶ lymph node cells with standard deviation of triplicates.

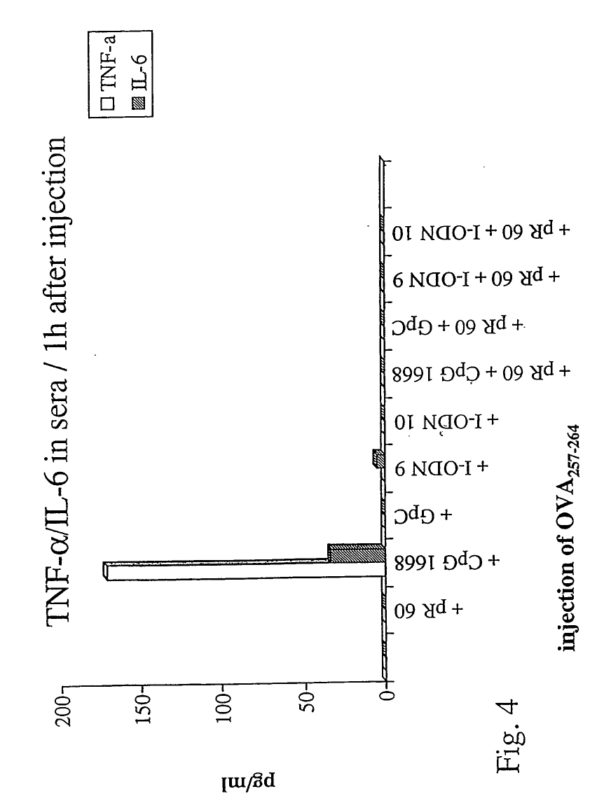

FIG. 4 shows the induction of systemic TNF-a production after the injection of OVA₂₅₇₋₂₆₄, poly-L-arginine (pR 60) and I-containing oligodeoxynucleotides (I-ODN), GpC or CpG 1668. Mice were injected into the hind footpads with mixtures as indicated. One hour after injection blood was taken from the tail vein and serum was prepared. The concentration of TNF-a and IL-6 in the sera was determined using cytokin-specific ELISAs.

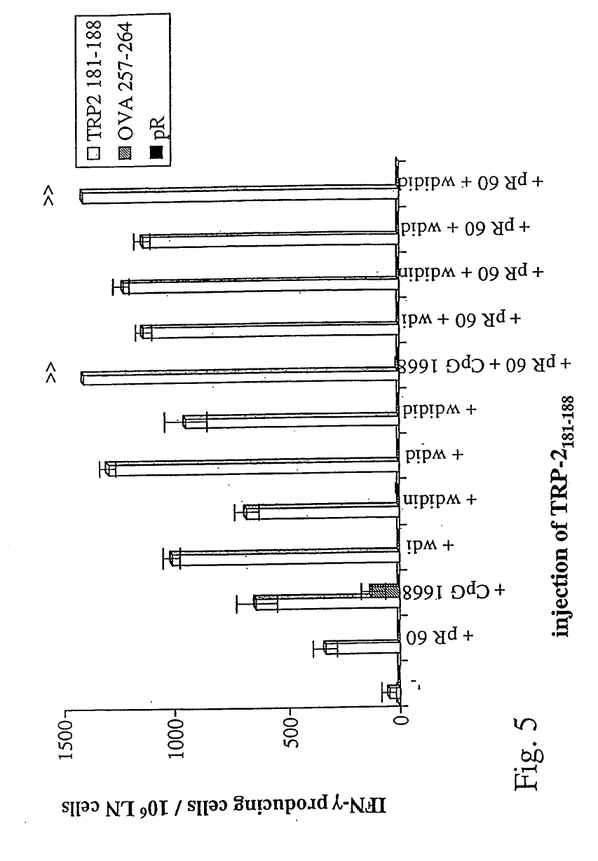

FIG. 5 shows the immune response against the Ovalbumin-derived peptide OVA₂₅₇₋₂₆₄ after the injection of TRP-2, poly-L-arginine, CpG 1668 or random 20-mer sequences containing deoxyinosine. Mice were injected into the hind footpads with mixtures as indicated. Four days later, draining lymph node cells were ex vivo stimulated with TRP-2, an irrelevant peptide OVA₂₅₇₋₂₆₄ or pR 60. The number of IFN-g producing cells was determined 24 hours later. using an ELISPOT assay. Results are expressed as the number of spots/1×10⁶ lymph node cells with standard deviation of triplicates.

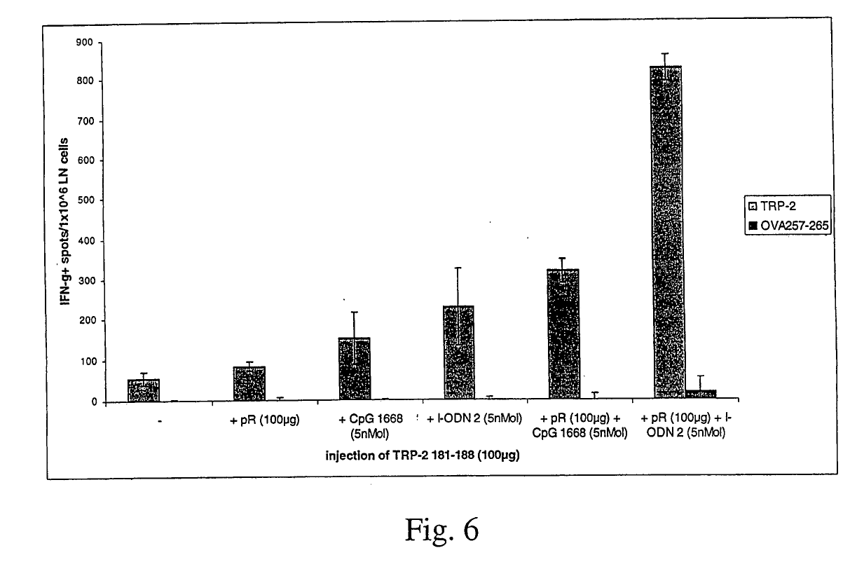

FIG. 6 shows the combined injection of I-ODN and poly-L-arginine (pR 60) together with a Melanoma-derived peptide.

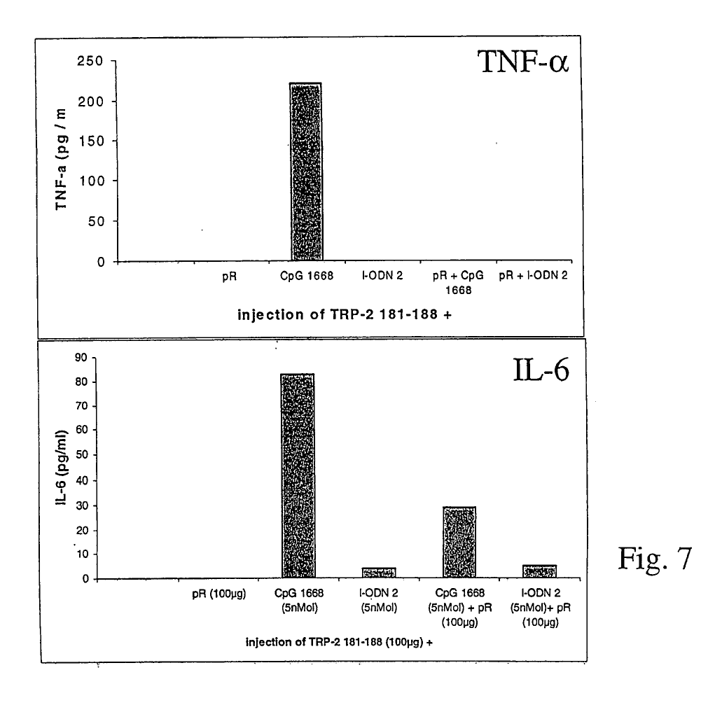

FIG. 7 shows that the combined injection of I-ODN and pR 60 together with a Melanoma-derived peptide reduces the induction of systemic TNF-α and IL-6.

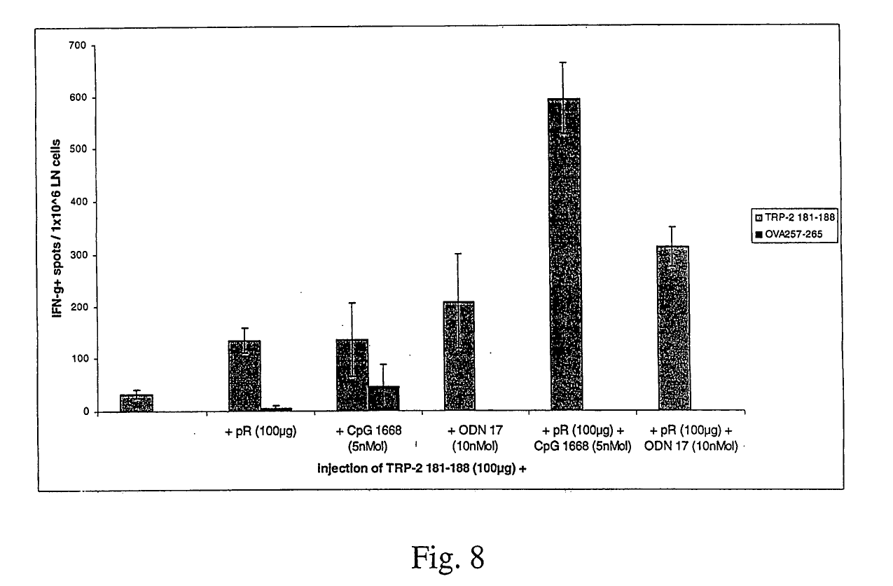

FIG. 8 shows the combined injection of a random 10-mer I-ODN and pR 60 together with a Melanoma-derived peptide.

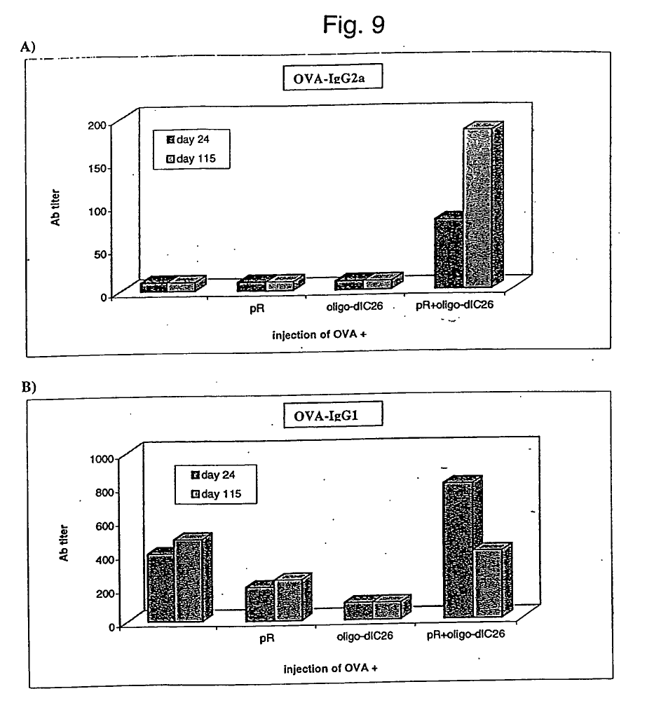

FIG. 9 shows that the combined application of ovalbumin (OVA) with oligo-dIC₂₆₋ₘₑᵣ and pR enhances production of OVA-specific IgG antibodies. Mice were injected subcutaneously into the footpad with mixtures as indicated. At day 24 and 115 after injection, sera were collected and screened by ELISA for OVA-specific IgG2a (A) and IgG1 (B) antibodies. The results are shown as the antibody titer.

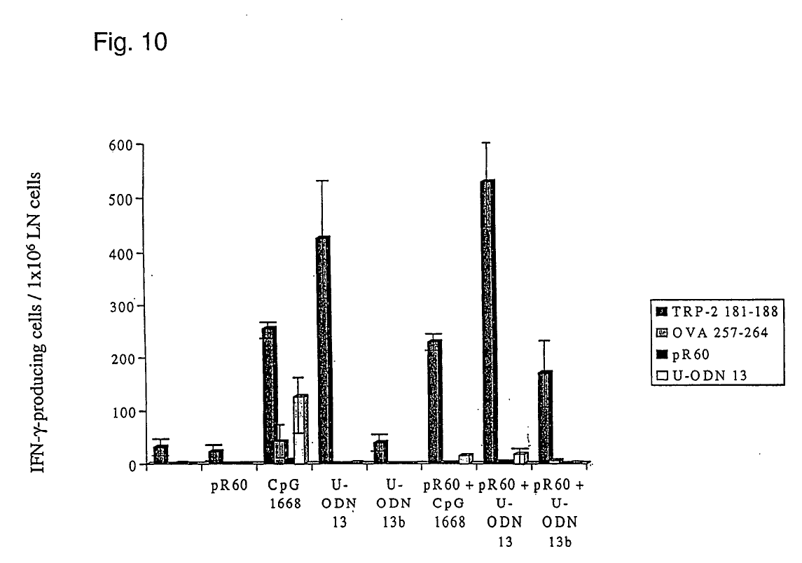

FIG. 10 shows that thiophosphate substituted deoxy-Uridin monophosphate modified oligodeoxynucleotides (U-ODN 13) induces in the presence or absence of poly-L-arginine a strong immune response against the melanoma-derived peptide TRP-2₁₈₁₋₁₈₈, which is higher than the immune response induced by CPG-ODN 1668 or CpG-ODN1668/poly-L-arginine. Furthermore, FIG. 1 shows that when U-ODNs, which are not substituted with thiophosphates (U-ODN 13b), were used only after co-injection of poly-L-arginine a strong peptide-specific immune response is induced. Mice were injected into the hind footpads with TRP-2₁₈₁₋₁₈₈, TRP-2₁₈₁₋₁₈₈ with either poly-L-arginine (pR60) or the U-containing oligodeoxynucleotide U-ODN 13/13b or with the combination of both, pR60 and U-ODN 13/13b. Four days later draining lymph node cells were ex vivo stimulated with TRP-2₁₈₁₋₁₈₈, an irrelevant peptide OVA₂₅₇₋₂₆₄, U-ODN 13/13b or pR60. The number of IFN-γ-producing cells was determined 24 hours later using an ELISPOT assay. Results are expressed as the number of IFN-γ-producing cells/1×10⁶ lymph node cells with standard deviation of triplicates.

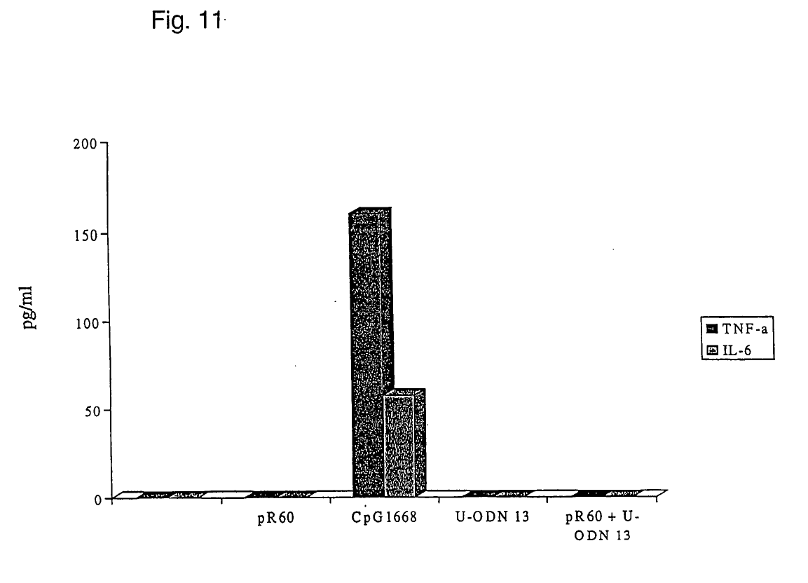

FIG. 11 shows that the deoxy-Uridin monophosphate modified oligodeoxynucleotide (U-ODN 13) does not induce the systemic production of TNF-α and IL-6. Mice were injected into the hind footpads with TRP-2₁₈₁₋₁₈₈, TRP-2₁₈₁₋₁₈₈ and poly-L-arginine or CpG 1668 or U-ODN 13, or TRP-2₁₈₁₋₁₈₈ and the combination of poly-L-arginine and U-ODN 13. One hour after injection blood was taken from the tail vein and serum was prepared. The amount of TNF-α and IL-6 in the sera was determined using ELISAs.

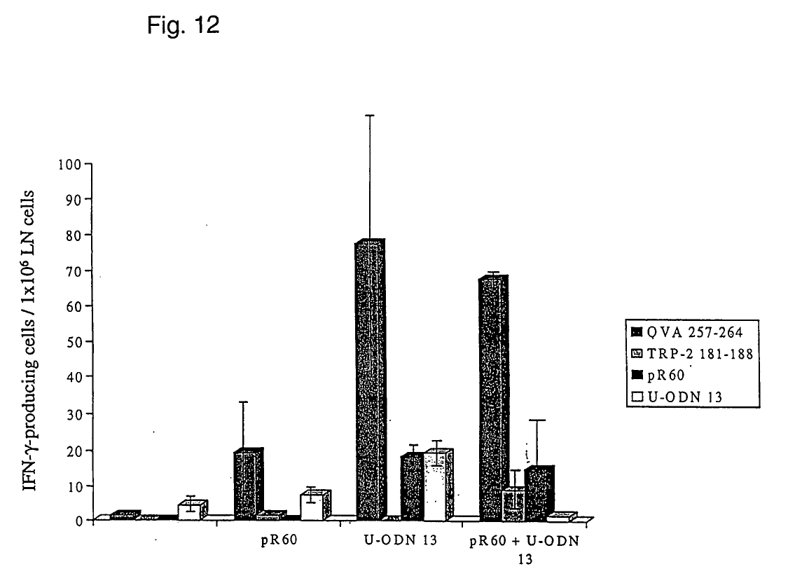

FIG. 12 shows that deoxy-Uridin monophosphate modified oligodeoxynucleotides (U-ODN 13) induces an immune response against the ovalbumin-derived peptide OVA₂₅₇₋₂₆₄ (SIINFEKL). Mice were injected into the hind footpads with OVA₂₅₇₋₂₆₄ alone, OVA₂₅₇₋₂₆₄ and poly-L-arginine (pR60) or the U-containing oligodeoxynucleotides U-ODN 13, or with OVA₂₅₇₋₂₆₄ and the combination of both, pR60 and U-ODN 13. Four days later, draining lymph node cells were ex vivo stimulated with OVA₂₅₇₋₂₆₄, an irrelevant peptide mTRP2₁₈₁₋₁₈₈ (murine tyrosinase related protein-2, VYDFFVWL), U-ODN 13 and pR 60. The number of IFN-γ producing cells was determined 24 hours later using an ELISPOT assay. Results are expressed as the number of IFN-γ-producing cells/1×10⁶ lymph node cells with standard deviation of duplicates.

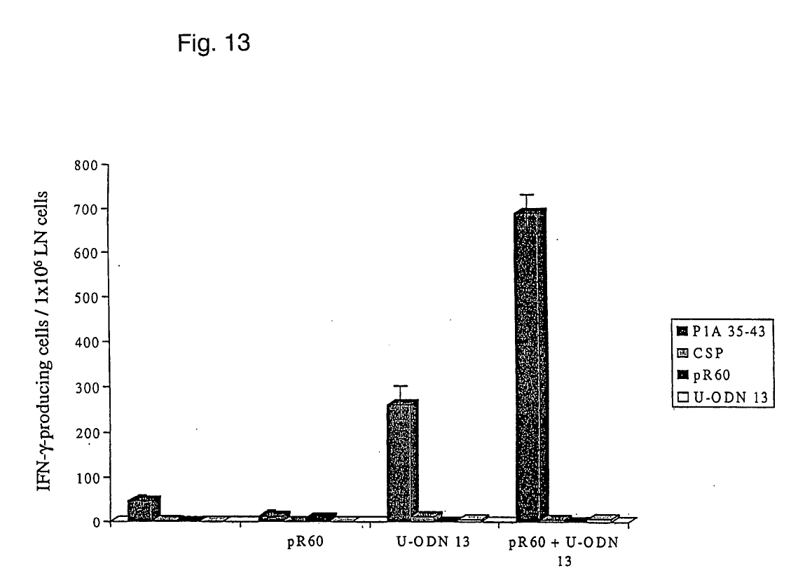

FIG. 13 shows that deoxy-Uridin monophosphate modified oligodeoxynucleotides (U-ODN 13) induces a strong immune response against the mouse mastocytoma-derived peptide P1A₃₅₋₄₃ (LPYLGWLVF), which can be further enhanced by co-injection of poly-L-arginine. Mice were injected into the hind footpads with P1A₃₅₋₄₃ alone, P1A₃₅₋₄₃ and poly-L-arginine or U-ODN 13, or with P1A₃₅₋₄₃ and the combination of both, pR60 and U-ODN 13. Four days later, draining lymph node cells were ex vivo stimulated with P1A₃₅₋₄₃, an irrelevant peptide CSP (SYVPSAEQI), U-ODN 13 and pR 60. The number of IFN-y producing cells was determined 24 hours later using an ELISPOT assay. Results are expressed as the number of IFN-γ-producing cells/1×10⁶ lymph node cells with standard deviation of triplicates.

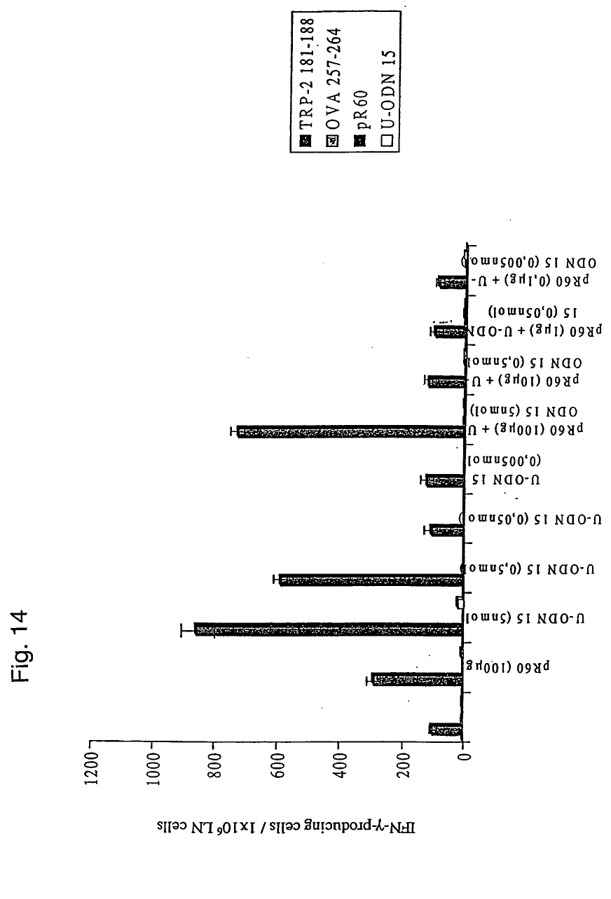

FIG. 14 shows that a cocktail of deoxy-Uridin monophosphate modified oligodeoxynucleotides (U-ODN 15) induces in the presence or absence of poly-L-arginine a strong immune response against the melanoma-derived peptide TRP-2₁₈₁₋₁₈₈. Mice were injected into the hind footpads with TRP-2_{181-188,} TRP-2₁₈₁₋₁₈₈ with either poly-L-arginine (pR60) or the U-containing oligodeoxynucleotide coktail UODN 15 or with the combination of both, pR60 and U-ODN 15. Four days later draining lymph node cells were ex vivo stimulated with TRP-2₁₈₁₋₁₈₈, an irrelevant peptide OVA₂₅₇₋₂₆₄, U-ODN 15 or pR60. The number of IFN-γ-producing cells was determined 24 hours later using an ELISPOT assay. Results are expressed as the number of IFN-γ-producing cells/1×10⁶ lymph node cells with standard deviation of triplicates.

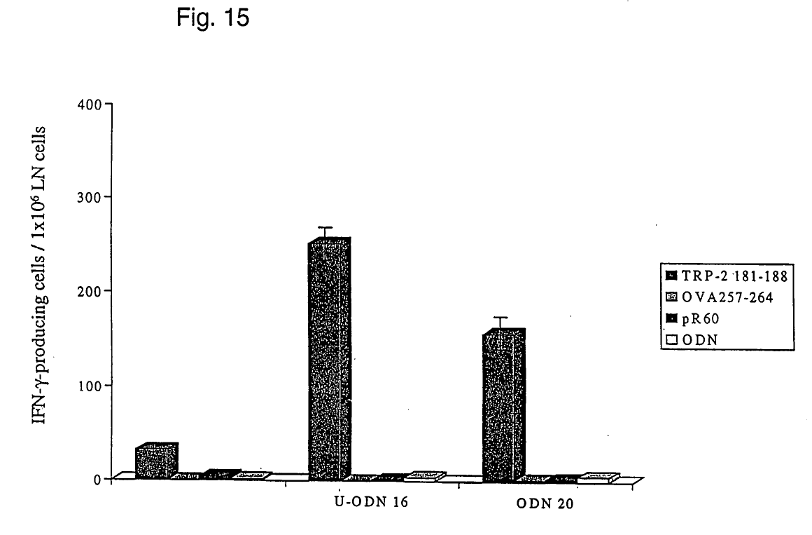

FIG. 15 shows that a cocktail of deoxy-Uridin monophosphate modified oligodeoxynucleotides (U-ODN 16) induces a strong immune response against the melanoma-derived peptide TRP-2₁₈₁₋₁₈₈, which is higher compared to the immune response after injection of TRP-2₁₈₁₋₁₈₈ alone or in combination with ODN 20, an oligonucleotide cocktail without deoxy-Uridin monophosphate. Mice were injected into the hind footpads with TRP-2₁₈₁₋₁₈₈, TRP-2₁₈₁₋₁₈₈ with either the U-containing oligodeoxynucleotide cocktail U-ODN 16 or the oligonucleotide cocktail ODN 20. Four days later draining lymph node cells were ex vivo stimulated with TRP-2₁₈₁₋₁₈₈, an irrelevant peptide OVA₂₅₇₋₂₆₄ U-ODN 16 or ODN 20. The number of IFN-γ-producing cells was determined 24 hours later using an ELISPOT assay. Results are expressed as the number of IFN-γ-producing cells/1×10⁶ lymph node cells with standard deviation of triplicates.

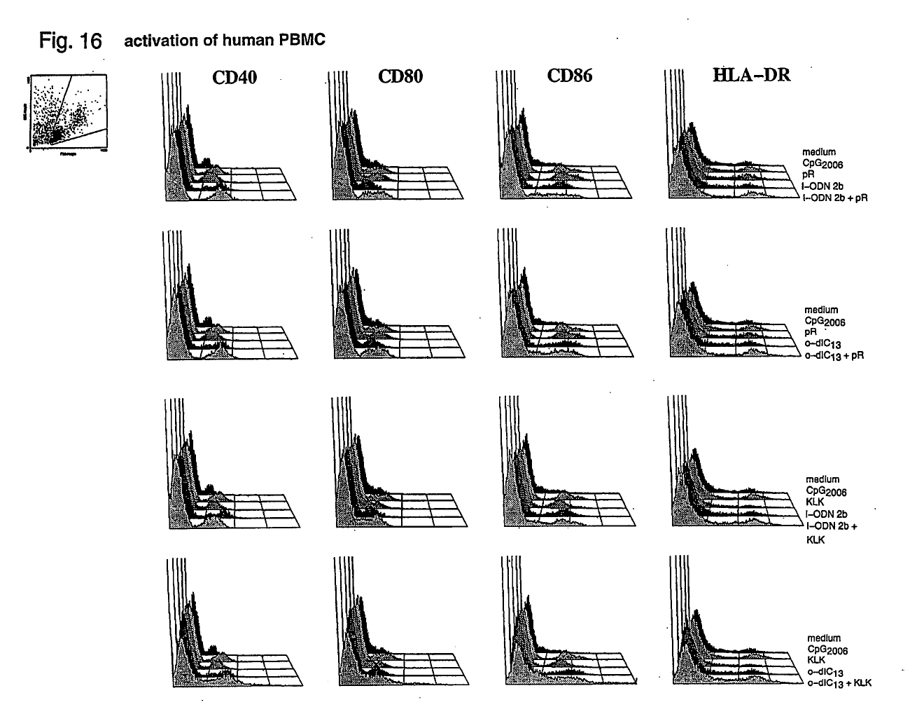

FIG. 16 shows the activation of human PBMC.

### EXAMPLES

In all experiments thiophosphate-substituted ODNs (with thiophosphate residues substituting for phosphate, hereafter called “thiophosphate substituted oligodeoxynucleotides”) were used since such ODNs display higher nuclease resistance (Ballas et al., 1996; Krieg et al., 1995; Parronchi et al., 1999).

### Example 1

The combined injection of different I-ODNs and poly-L-arginine (pR 60) synergistically enhances the immune response against an Ovalbumin-derived peptide.

| | |
|---|---|
| Mice | C57BI/6 (Harlan/Olac) |
| Peptide | OVA₂₅₇₋₂₆₄-Peptide (SIINFEKL), a MHC |
| | class I (H-2Kb)-restricted |
| | epitope of chicken ovalbumin |
| | (Rotzschke et al., 1991), was |
| | synthesized using standard solid |
| | phase F-moc chemistry synthesis, |
| | HPLC purified and analysed by |
| | mass spectroscopy for purity. |
| | Dose: 300 mg/mouse |
| Poly-L-arginine60 (pR60) | Poly-L-arginine with an average |
| | degree of polymerization of 60 |
| | arginine residues; SIGMA chemi- |
| | cals |
| | Dose: 100 mg/mouse |
| CpG-ODN 1668 | thiophosphate substituted ODNs |
| | containing a CpG motif: |
| | tcc atg acg ttc ctg atg ct, were |
| | synthesized by NAPS GmbH, Göttin- |
| | gen. |
| | Dose: 5 nmol/mouse |
| I-ODN 1 | thiophosphate substituted ODNs |
| | containing deoxyinosine: |
| | tcc ati aci ttc ctg atg ct, were |
| | synthesized by NAPS GmbH, Gottin- |
| | gen. |
| | Dose: 5 nmol/mouse |
| I-ODN 2 | thiophosphate substituted ODNs |
| | containing deoxyinosine: |
| | tcc atg aci ttc ctg atg ct, were |
| | synthesized by NAPS GmbH, Göttin- |
| | gen. |
| | Dose: 5 nmol/mouse |
| I-ODN 3 | thiophosphate substituted ODNs |
| | containing deoxyinosine: |
| | tcc ati aci ttc cti ati ct, were |
| | synthesized by NAPS GmbH, Göttin- |
| | gen. |
| | Dose: 5 nmol/mouse |

Experimental groups (5 mice per group)
1. OVA₂₅₇₋₂₆₄
2. OVA₂₅₇₋₂₆₄+pR 60
3. OVA₂₅₇₋₂₆₄+CpG 1668
4. OVA₂₅₇₋₂₆₄+I-ODN 1
5. OVA₂₅₇₋₂₆₄+I-ODN 2
6. OVA₂₅₇₋₂₆₄+I-ODN 3
7. OVA₂₅₇₋₂₆₄+CpG 1668+pR 60
8. OVA₂₅₇₋₂₆₄+I-ODN 1+pR 60
9. OVA₂₅₇₋₂₆₄+I-ODN 2+PR 60
10. OVA₂₅₇₋₂₆₄+I-ODN 3+PR 60

On day 0 mice were injected into each hind footpad with a total volume of 100 ml (50 ml per footpad) containing the above mentioned compounds. Animals were sacrificed 4 days after injection and popliteal lymph nodes were harvested. Lymph nodes were passed through a 70 mm cell strainer and washed twice with DMEM medium (GIBCO BRL) containing 5% fetal calf serum (FCS, SIGMA chemicals). Cells were adjusted to 3×10⁶cells/ml in DMEM/5%/FCS. An IFN-g ELISPOT assay was carried out in triplicates as described (Miyahira et al., 1995). This method is a widely used procedure allowing the quantification of antigen-specific T cells. Lymphocytes were stimulated ex vivo with medium background-control, OVA₂₅₇₋₂₆₄-peptide or Concanavalin A (Con A). Spots representing single IFN-g producing T cells were counted and the number of background spots was substracted from all samples. The high number of spots detected after the stimulation with Con A (data not shown) indicate a good condition of the used lymphocytes. For each experimental group of mice the number of spots/1×10⁶ cells are illustrated in FIG. 1.

One hour after injection blood was taken from the tail vein and serum was prepared to determine the induction of systemic TNF-a using an ELISA (FIG. 2).

### Example 2

The exchange of Guanosine by desoxy-Inosine converts the non-immunogeneic GpC-sequence to a highly immunogeneic one, especially when combined with poly-L-arginine (pR60).

| | |
|---|---|
| Mice | C57Bl/6 (Harlan/Olac) |
| Peptide | OVA₂₅₇₋₂₆₄-Peptide (SIINFEKL), a MHC class I |
| | (H-2Kb)-restricted epitope of chicken |
| | ovalbumin (Rotzschke et al., 1991), was |
| | synthesized using standard solid phase |
| | F-moc synthesis, HPLC purified and analysed |
| | by mass spectroscopy for purity. |
| | Dose: 300 μg/mouse |
| Poly-L-arginine 60 | (pR60) Poly-L-arginine with an average |
| | degree of polymerization of 60 arginine |
| | residues; SIGMA chemicals |
| | Dose: 100 μg/mouse |
| CpG-ODN 1668 | thiophosphate substituted ODNs containing a |
| | CpG motif: tcc atg acg ttc ctg atg ct, were |
| | synthesized by NAPS GmbH, Göttingen. |
| | Dose: 5 nmol/mouse |
| GpC-ODN | thiophosphate substituted ODNs containing an |
| | non-immunogeneic GpC motif: tcc atg agc ttc |
| | ctg atg ct were synthesized by NAPS GmbH, |
| | Göttingen. |
| | Dose: 5 nmol/mouse |
| I-ODN 9 | thiophosphate substituted ODNs containing |
| | deoxyinosine: tcc atg aic ttc ctg atg ct were |
| | synthesized by NAPS GmbH, Göttingen. |
| | Dose: 5 nmol/mouse |
| I-ODN 10 | thiophosphate substituted ODNs containing |
| | deoxyinosine: tcc ati aic ttc cti ati ct were |
| | synthesized by NAPS GmbH, Göttingen. |
| | Dose: 5 nmol/mouse |

Experimental groups (5 mice per group)
OVA₂₅₇₋₂₆₄
OVA₂₅₇₋₂₆₄+pR 60
OVA₂₅₇₋₂₆₄+CpG 1668
OVA₂₅₇₋₂₆₄+GpC
OVA₂₅₇₋₂₆₄+I-ODN 9
OVA₂₅₇₋₂₆₄+I-ODN 10
OVA₂₅₇₋₂₆₄+CpG 1668+pR 60
OVA₂₅₇₋₂₆₄+GpC+pR 60
OVA₂₅₇₋₂₆₄+I-ODN 9+pR 60
OVA₂₅₇₋₂₆₄+I-ODN 10+pR 60

On day 0 mice were injected into each hind footpad with a total volume of 100 μl (50 μl per footpad) containing the above mentioned compounds. Animals were sacrificed 4 days after injection and popliteal lymph nodes were harvested. Lymph nodes were passed through a 70 μm cell strainer and washed twice with DMEM medium (GIBCO BRL) containing 5% fetal calf serum (FCS, SIGMA chemicals). Cells were adjusted to 3×10⁶cells/ml in DMEM/5%FCS. An IFN-g ELISPOT assay was carried out in triplicates as described (Miyahira et al., 1995). This method is a widely used procedure allowing the quantification of antigen-specific T cells. Lymphocytes were stimulated ex vivo in triplicates with medium (background), OVA₂₅₇₋₂₆₄-peptide, an irrelevant peptide mTRP-2₁₈₁₋₁₈₈ (murine tyrosinase related protein-2, VYDFFVWL), pR 60 and Concanavalin A (Con A). Spots representing single IFN-g producing T cells were counted and the number of background spots was substracted from all samples. The high number of spots detected after the stimulation with Con A (data not shown) indicate a good condition of the used lymphocytes. For each experimental group of mice the number of spots/1×10⁶ cells are illustrated in FIG. 3, the standard deviation of ex vivo-stimulated triplicates are given. One hour after injection blood was taken from the tail vein and serum was prepared to determine the induction of systemic TNF-a and IL-6 using cytokine-specific ELISAs (FIG. 4).

### Example 3

The combined injection of random 20-mer sequences containing de- oxyinosine and a Melanoma-derived peptide induces a strong immune response against the peptide which can be further enhanced by the co-application of poly-L-arginine (pR 60).

| | |
|---|---|
| Mice | C57Bl/6 (Harlan/Olac) |
| Peptide | TRP-2-peptide (VYDFFVWL), a MHC class I |
| | (H-2K^{b})-restricted epitope of mouse |
| | tyrosinase related protein-2 (Bllom et al., |
| | 1997) was synthesized by standard solid phase |
| | F-moc synthesis, HPLC purified and analyzed |
| | by mass spectroscopy for purity. |
| | Dose: 300 μg/mouse |
| Poly-L-arginine 60 | (pR60) Poly-L-arginine with an average |
| | degree of polymerization of 60 arginine |
| | residues; SIGMA chemicals |
| | Dose: 100 μg/mouse |
| CpG-ODN 1668 | thiophosphate substituted ODNs containing a |
| | CpG motif: tcc atg acg ttc ctg atg ct, were |
| | synthesized by NAPS GmbH, Göttingen. |
| | Dose: 5 nmol/mouse |
| wdi | thiophosphate substituted ODNs: nhh hhh wdi |
| | nhh hhh hhh wn were synthesized by NAPS GmbH, |
| | Göttingen. |
| | Dose: 5 nmol/mouse |
| wdidin | thiophosphate substituted ODNs: nhh hhh wdi |
| | nhh hhh hhh wn were synthesized by NAPS GmbH, |
| | Göttingen. |
| | Dose: 5 nmol/mouse |
| wdid | thiophosphate substituted ODNs: nhh hhh wdi |
| | dhh hhh hhh wn were synthesized by NAPS GmbH, |
| | Göttingen. |
| | Dose: 5 nmol/mouse |
| wdidid | thiophosphate substituted ODNs: nhh wdi did |
| | hhh hdi ddi dh were synthesized by NAPS GmbH, |
| | Göttingen. |
| | Dose: 5 nmol/mouse |

Experimental groups (5 mice per group)
1. TRP-2
2. TRP-2+pR 60
3. TRP-2+CpG 1668
4. TRP-2+wdi
5. TRP-2+wdidin
6. TRP-2+wdid
7. TRP-2+wdidid
8. TRP-2+CpG 1668+pR 60
9. TRP-2+wdi+pR 60
10. TRP-2+wdidin+pR 60
11. TRP-2+wdid+PR 60
12. TRP-2+wdidid+pR 60

On day 0 mice were injected into each hind footpad with a total volume of 100 μl (50 μl per footpad) containing the above mentioned compounds. Animals were sacrificed 4 days after injection and popliteal lymph nodes were harvested. Lymph nodes were passed through a 70μm cell strainer and washed twice with DMEM medium (GIBCO BRL) containing 5% fetal calf serum (FCS, SIGMA chemicals). Cells were adjusted to 3xlO⁶cells/ml in DMEM/5%FCS. An IFN-g ELISPOT assay was carried out in triplicates as described (Miyahira et al., 1995). This method is a widely used procedure allowing the quantification of antigen-specific T cells. Lymphocytes were stimulated ex vivo in triplicates with medium (background), TRP-2-peptide, an irrelevant OVA₂₅₇₋₂₆₄-peptide, pR 60 and Concanavalin A (Con A). Spots representing single IFN-g producing T cells were counted and the number of background spots was substracted from all samples. The high number of spots detected after the stimulation with Con A (data not shown) indicate a good condition of the used lymphocytes. For each experimental group of mice the number of spots/1×10 cells are illustrated in FIG. 5, the standard deviation of ex vivo-stimulated triplicates are given.

### Example 4

The combined injection of I-ODN and poly-L-arginine (pR 60) synergistically enhances the immune response against a Melanoma-derived peptide.

Experimental groups (5 mice per group)

1. TRP-2₁₈₁₋₁₈₈
2. TRP-2₁₈₁₋₁₈₈+pR 60
3. TRP-2₁₈₁₋₁₈₈+CpG 1668
4. TRP-2₁₈₁₋₁₈₈+I-ODN 2
5. TRP-2₁₈₁₋₁₈₈+CpG 1668+pR 60
6. TRP-2₁₈₁₋₁₈₈+I-ODN 2+pR 60

On day 0 mice were injected into each hind footpad with a total volume of 100 μl (50 μl per footpad) containing the above mentioned compounds. Animals were sacrificed 4 days after injection and popliteal lymph nodes were harvested. Lymph nodes were passed through a 70 μm cell strainer and washed twice with DMEM medium (GIBCO BRL) containing 5% fetal calf serum (FCS, SIGMA chemicals). Cells were adjusted to 3×10⁶cells/ml in DMEM/5%/FCS. An IFN-γ ELISPOT assay was carried out in triplicates as described (Miyahira et al., 1995). This method is a widely used procedure allowing the quantification of antigen-specific T cells. Lymphocytes were stimulated ex vivo in triplicates with medium background-control, TRP-2₁₈₁₋₁₈₈-peptide, an irrelevant OVA₂₅₇₋₂₆₄-peptide and Concanavalin A (Con A). Spots representing single IFN-γ producing T cells were counted and the number of background spots was substracted from all samples. The high number of spots detected after the stimulation with Con A (data not shown) indicate a good condition of the used lymphocytes. For each experimental group of mice the number of spots/1×10⁶ cells are illustrated in FIG. 6, the standard deviation of ex vivo-stimulated triplicates are given.

One hour after injection blood was taken from the tail vein and serum was prepared to determine the induction of systemic TNF-α and IL-6 using specific ELISAs (FIG. 7).

### Example 5

The combined injection of random 10-mer I-ODN and poly-L-arginine (pR 60) synergistically enhances the immune response against a Melanoma-derived peptide.

Exoerimental groups (5 mice per group)

1. TRP-2₁₈₁₋₁₈₈
2. TRP-2₁₈₁₋₁₈₈+pR 60
3. TRP-2₁₈₁₋₁₈₈+CpG 1668
4. TRP-2₁₈₁₋₁₈₈+ODN 17
5. TRP-2₁₈₁₋₁₈₈+CpG 1668+pR 60
6. TRP-2₁₈₁₋₁₈₈+ODN 17+pR 60

On day 0 mice were injected into each hind footpad with a total volume of 100 μl (50 μl per footpad) containing the above mentioned compounds. Animals were sacrificed 4 days after injection and popliteal lymph nodes were harvested. Lymph nodes were passed through a 70 μm cell strainer and washed twice with DMEM medium (GIBCO BRL) containing 5% fetal calf serum (FCS, SIGMA chemicals). Cells were adjusted to 3×10⁶cells/ml in DMEM/5%/FCS. An IFN-γ ELISPOT assay was carried out in triplicates as described Miyahira et al., 1995). This method is a widely used procedure allowing the quantification of antigen-specific T cells. Lymphocytes were stimulated ex vivo in triplicates with medium back-ground-control, TRP-2₁₈₁₋₁₈₈-peptide, an irrelevant OVA₂₅₇₋₂₆₄-peptide and Concanavalin A (Con A). Spots representing single IFN-y producing T cells were counted and the number of background spots was substracted from all samples. The high number of spots detected after the stimulation with Con A (data not shown) indicate a good condition of the used lymphocytes. For each experimental group of mice the number of spots/1×10⁶ cells are illustrated in FIG. 8, the standard deviation of ex vivo-stimulated triplicates are given.

| | |
|---|---|
| Mice | C57Bl/6 (Harlan/Olac) |
| Peptide | TRP-2-peptide (VYDFFVWL), a MHC |
| | class I (H-2K^{b})-restricted epi- |
| | tope of mouse tyrosinase related |
| | protein-2 (Bllom et al., 1997) |
| | was synthesized by standard solid |
| | phase F-moc synthesis, HPLC puri- |
| | fied and analyzed by mass spec- |
| | troscopy for purity. |
| | Dose: 100 μg/mouse |
| Poly-L-arginine60 (pR60) | Poly-L-arginine with an average |
| | degree of polymerization of 60 |
| | arginine residues; SIGMA chemi- |
| | cals |
| | Dose: 100 μg/mouse |
| CpG-ODN 1668 | thiophosphate substituted ODNs |
| | containing a CpG motif: |
| | tcc atg acg ttc ctg atg ct, were |
| | synthesized by NAPS GmbH, Göttin- |
| | gen. |
| | Dose: 5 nmol/mouse |
| ODN 17 | thiophosphate substituted ODNs |
| | containing deoxyinosine: |
| | hhh wdi dhh h, were synthesized |
| | by NAPS GmbH, Göttingen. |
| | (h = CAT, w = AT, d = GAT) |
| | Dose: 10 nmol/mouse |
| Mice | C57Bl/6 (Harlan/Olac) |
| Peptide | TRP-2-peptide (VYDFFVWL), a MHC |
| | class I (H-2K^{b})-restricted epi- |
| | tope of mouse tyrosinase related |
| | protein-2 (Bllom et al., 1997) |
| | was synthesized by standard solid |
| | phase F-moc synthesis, HPLC puri- |
| | fied and analyzed by mass spec- |
| | troscopy for purity. |
| | Dose: 100 μg/mouse |
| Poly-L-arginine60 (pR60) | Poly-L-arginine with an average |
| | degree of polymerization of 60 |
| | arginine residues; SIGMA chemi- |
| | cals |
| | Dose: 100 μg/mouse |
| CpG-ODN 1668 | thiophosphate substituted ODNs |
| | containing a CpG motif: |
| | tcc atg acg ttc ctg atg ct, were |
| | synthesized by NAPS GmbH, Göttin- |
| | gen. |
| | Dose: 5 nmol/mouse |
| I-ODN 2 | thiophosphate substituted ODNs |
| | containing deoxyinosine: |
| | tcc atg aci ttc ctg atg ct, were |
| | synthesized by NAPS GmbH, Göttin- |
| | gen. |
| | Dose: 5 nmol/mouse |

### Example 6

The combined application of oligo-deoxyIC₂₆₋ₘₑᵣ and poly-L-arginine (pR) enhances the ovalbumin (OVA)-specific humoral response.

| | |
|---|---|
| Mice | C57Bl/6 (Harlan/Olac) |
| Ovalbumin (OVA) | Ovalbumin from chicken egg, grade V, |
| | SIGMA Chemicals, A-5503, Lot 54H7070 |
| | Dose: 50 μg/mouse |
| Poly-L-arginine (pR) | Poly-L-arginine with an average de- |
| | gree of polymerization of 60 ar- |
| | ginine residues; SIGMA Chemicals, |
| | P-4663, Lot 68H5903 |
| | Dose: 100 μg/mouse |
| Oligo-deoxy IC, 26-mer | oligo-dIC₂₆₋ₘₑᵣ was synthesized by |
| (oligo-dIC₂₆₋ₘₑᵣ) | standard phosphoamidide chemistry on |
| | a 4 μmol scale and purified by HPLC |
| | (NAPS Göttingen, Germany) |
| | Dose: 5 nmol/mouse |

Experimental groups (4 mice per group)
1. OVA+oligo-dIC₂₆₋ₘₑᵣ+pR
2. OVA+oligo-dIC₂₆₋ₘₑᵣ
3. OVA+pR
4. OVA

On day 0, mice were injected into each hind footpad with a total volume of 100 μl (50 μl per footpad) containing the above listed compounds. On day 24 after injection, serum was collected and screened by ELISA for the presence of OVA-specific antibodies. These results show that the injection of OVA in combination with oligo-dIC and pR enhanced the production of OVA-specific IgG antibodies when compared with injection of OVA with each of the substances alone (FIG. 13A, B). Interestingly, titers of both IgG2a and IgG1 were increased upon one single injection of OVA with oligo-dIC/pR, implying that both Thl and Th2 cells were involved. However, after 115 days only the increased IgG2a levels were still detectable in sera of mice injected with OVA and oligo-dIC/pR.

These data demonstrate that the combined injection of OVA with oligo-dIC and pR enhances the OVA-specific humoral response. This response is characterized by the production of both Th1- and Th2-induced antibody isotypes in the early phase, but later, mainly by Th1-induced antibodies.

### Example 7

Generation of specific immune responses against a melanoma-derived peptide (TRP-2₁₈₁₋₁₈₈) with deoxy-Uridine monophosphate modified oligonucleotide U-ODN 13.

| | |
|---|---|
| Mice | C57BI/6 (Harlan/Olac) |
| Peptide | TRP-2-peptide (VYDFFVWL), a MHC |
| | class I (H-2Kb)-restricted epi- |
| | tope of mouse tyrosinase related |
| | protein-2 (B16 melanoma, Bloom, |
| | M. B. et al., J Exp. Med 1997, |
| | 185, 453-459), synthesized by |
| | standard solid phase F-moc syn- |
| | thesis, HPLC purified and ana- |
| | lysed by mass spectroscopy for |
| | purity |
| | Dose: 100 μg/mouse |
| Poly-L-arginine 60 (pR60) | Poly-L-arginine with an average |
| | degree of polymerization of 60 |
| | arginine residues; SIGMA chemi- |
| | cals |
| | Dose: 100 μg/mouse |
| CpG 1668 | thiophosphate substituted ODNs |
| | containing CpG-motif: |
| | tcc atg acg ttc ctg atg ct, were |
| | synthesized by NAPS GmbH, Göttin- |
| | gen. |
| | Dose: 5 nmol/mouse |
| U-ODN 13 | thiophosphate substituted ODNs |
| | containing deoxy-Uridine mono- |
| | phosphate: |
| | tcc atg acu ttc ctg atg ct, were |
| | synthesized by NAPS GmbH, Gottin- |
| | gen. |
| | Dose: 5 nmol/mouse |
| U-ODN 13b | ODNs containing deoxy-Uridine |
| | monophosphate (not substituted |
| | with thiophospate): |
| | tcc atg acu ttc ctg atg ct, were |
| | synthesized by NAPS GmbH, Göttin- |
| | gen. |
| | Dose: 5 nmol/mouse |

Experimental groups (4 mice per group)
1. TRP-2₁₈₁₋₁₈₈
2. TRP-2₁₈₁₋₁₈₈+pR 60
3. TRP-2₁₈₁₋₁₈₈+CpG-ODN
4. TRP-2₁₈₁₋₁₈₈+U-ODN 13
5. TRP-2₁₈₁₋₁₈₈+U-ODN 13b
6. TRP-2₁₈₁₋₁₈₈+CPG-ODN +pR 60
7. TRP-2₁₈₁₋₁₈₈+U-ODN 13+pR 60
8. TRP-2₁₈₁₋₁₈₈+U-ODN 13b+pR 60

On day 0 mice were injected into each hind footpad with a total volume of 100 μl (50 μl per footpad) containing the above-mentioned compounds. Animals were sacrificed 4 days after injection and popliteal lymph nodes were harvested. Lymph nodes were passed through a 70 μm cell strainer and washed twice with DMEM medium (GIBCO BRL) containing 5% fetal calf serum (FCS, SIGMA chemicals). Cells were adjusted to the appropriate cell number in DMEM/5%/FCS. An IFN-γ ELISPOT assay was carried out in triplicates as described (Miyahira et al., 1995). This method is a widely used procedure allowing the quantification of antigen-specific T cells. Lymphocytes were stimulated ex vivo in triplicates with medium (background-control), TRP-2₁₈₁₋₁₈₈-peptide, an irrelevant peptide OVA₂₅₇₋₂₆₄, pR 60, U-ODN13 and Concanavalin A (Con A). Spots representing single IFN-γ producing T cells were counted and the number of background spots was substracted from all samples. The high number of spots detected after the stimulation with Con A (data not shown) indicates a good condition of the used lymphocytes. For each experimental group of mice the number of IFN-γ-producing cells/1×10⁶ cells are illustrated in FIG. 10, the standard deviation of ex vivo-stimulated triplicates is given.

This experiment shows that the injection of TRP-2₁₈₁₋₁₈₈ (hydrophobic peptide) with thiophosphate substituted U-ODNs strongly enhances TRP-2₁₈₁₋₁₈₈-specific immune responses compared to the injection of TRP-2₁₈₁₋₁₈₈ alone. Interestingly, compared to the injection of TRP-2₁₈₁₋₁₈₈/CpG-ODN, higher number of TRP-2₁₈₁₋₁₈₈-specific T cells are induced by injection of TRP-2₁₈₁₋₁₈₈/U-ODN 13. The co-injection of poly-L-arginine does not influence this strong response. In contrast, when U-ODN 13b, which is not substituted with thiophosphates, was used, only upon co-injection of poly-L-arginine a high immune response was induced.

### Example 8

Application of deoxy-Uridine monophosphate modified oligodeoxynucleotides does not induce the production of pro-inflammatory cytokines

| | |
|---|---|
| Mice | C57BI/6 (Harlan/Olac) |
| Peptide | TRP-2-peptide (VYDFFVWL), a MHC |
| | class I (H-2Kb)-restricted epi- |
| | tope of mouse tyrosinase related |
| | protein-2 (B16 melanoma, Bloom, |
| | M. B. et al., J Exp. Med 1997, |
| | 185, 453-459), synthesized by |
| | standard solid phase F-moc syn- |
| | thesis, HPLC purified and ana- |
| | lysed by mass spectroscopy for |
| | purity |
| | Dose: 100 μg/mouse |
| Poly-L-arginine 60 (pR60) | Poly-L-arginine with an average |
| | degree of polymerization of 60 |
| | arginine residues; SIGMA chemi- |
| | cals |
| | Dose: 100 μg/mouse |
| CpG 1668 | thiophosphate substituted ODNs |
| | containing a CpG motif: |
| | tcc atg acg ttc ctg atg ct, were |
| | synthesized by NAPS GmbH, Göttin- |
| | gen. |
| | Dose: 5 nmol/mouse |
| U-ODN 13 | thiophosphate substituted ODNs |
| | containing deoxy-Uridine mono- |
| | phosphate: |
| | tcc atg acu ttc ctg atg ct, were |
| | synthesized by NAPS GmbH, Göttin- |
| | gen. |
| | Dose: 5 nmol/mouse |

Experimental groups (4 mice per group)
1. TRP-2₁₈₁₋₁₈₈
2. TRP-2₁₈₁₋₁₈₈+pR 60
3. TRP-2₁₈₁₋₁₈₈+CpG 1668
4. TRP-2₁₈₁₋₁₈₈+U-ODN 13
5. TRP-2₁₈₁₋₁₈₈+U-ODN 13+pR 60

On day 0 mice were injected into each hind footpad with a total volume of 100 μl (50 μl per footpad) containing the above-mentioned compounds. One hour after injection blood was taken via the tail vein and serum was prepared. The amount of TNF-α and IL-6 in the sera were determined by specific ELISAs.

FIG. 11 shows that, in contrast to the application of CPG-ODN 1668 the application of U-ODN 13 in combination with a peptide does not induce the systemic production of pro-inflammatory cytokines.

### Example 9

Generation of specific immune responses against an allergen derived peptide with deoxy-Uridine monophosphate modified oligonucleotide U-ODN 13.

| | |
|---|---|
| Mice | C57BI/6 (Harlan/Olac) |
| Peptide | OVA₂₅₇₋₂₆₄-Peptide (SIINFEKL), a MHC |
| | class I (H-2Kb)-restricted |
| | epitope of chicken ovalbumin |
| | (Rotzschke et al., 1991), was |
| | synthesized using standard solid |
| | phase F-moc chemistry synthesis, |
| | HPLC purified and analysed by |
| | mass spectroscopy for purity. |
| | Dose: 300 μg/mouse |
| Poly-L-arginine 60 (pR60) | Poly-L-arginine with an average |
| | degree of polymerization of 60 |
| | arginine residues; SIGMA chemi- |
| | cals |
| | Dose: 100 μg/mouse |
| U-ODN 13 | thiophosphate substituted ODNs |
| | containing deoxy-Uridine mono- |
| | phosphate: |
| | tcc atg acu ttc ctg atg ct, were |
| | synthesized by NAPS GmbH, Göttin- |
| | gen. |
| | Dose: 5 nmol/mouse |

Experimental groups (4 mice per group)
1. OVA2₂₅₇₋₂₆₄
2. OVA2₂₅₇₋₂₆₄+pR 60
3. OVA2₂₅₇₋₂₆₄+U-ODN 13
4. OVA2₂₅₇₋₂₆₄+U-ODN 13+pR 60

On day 0 mice were injected into each hind footpad with a total volume of 100 μl (50 μl per footpad) containing the above-mentioned compounds. Animals were sacrificed 4 days after injection and popliteal lymph nodes were harvested. Lymph nodes were passed through a 70 μm cell strainer and washed twice with DMEM medium (GIBCO BRL) containing 5% fetal calf serum (FCS, SIGMA chemicals). Cells were adjusted to the appropriate cell number in DMEM/5%/FCS. An IFN-γ ELISPOT assay was carried out in duplicates as described (Miyahira et al., 1995). This method is a widely used procedure allowing the quantification of antigen-specific T cells. Lymphocytes were stimulated ex vivo in duplicates with medium (background-control), OVA₂₅₇₋₂₆₄peptide, an irrelevant peptide TRP-2₁₁₈₋₁₈₈, pR 60, U-ODN13 and Concanavalin A (Con A). Spots representing single IFN-γ producing T cells were counted and the number of background spots was substracted from all samples. The high number of spots detected after the stimulation with Con A (data not shown) indicates a good condition of the used lymphocytes. For each experimental group of mice the number of IFN-γ-producing cells/1×10⁶ cells are illustrated in FIG. 12, the standard deviation of ex vivo-stimulated duplicates is given.

This experiment shows that deoxy-Uridine monophosphat modified ODNs also induces an immune response against a hydrophilic peptide (OVA₂₅₇₋₂₆₄). The co-injection of poly-L-arginine has no influence on this immune response.

### Example 10

Generation of specific immune responses against a mastocytoma-derived peptide with deoxy-Uridine monophosphate modified oligonucleotide U-ODN 13.

| | |
|---|---|
| Mice | C57BI/6 (Harlan/Olac) |
| Peptide | Mouse mastocytoma P815-derived |
| | Peptide P1A (LPYLGWLVF), re- |
| | stricted to MHC class I (H2-Ld) |
| | (Lethe et al., 1992). |
| | Dose: 100 μg/mouse |
| Poly-L-arginine 60 (pR60) | Poly-L-arginine with an average |
| | degree of polymerization of 60 |
| | arginine residues; SIGMA chemi- |
| | cals |
| | Dose: 100 μg/mouse |
| U-ODN 13 | thiophosphate substituted ODNs |
| | containing deoxy-Uridine mono- |
| | phosphate: |
| | tcc atg acu ttc ctg atg ct, were |
| | synthesized by NAPS GmbH, Göttin- |
| | gen. |
| | Dose: 5 nmol/mouse |

Experimental groups (4 mice per group)
1. P1A₃₅₋₄₃
2. P1A₃₅₋₄₃+pR 60
3. P1A₃₅₋₄₃+U-ODN 13
4. P1A₃₅₋₄₃+U-ODN 13+pR 60

On day 0 mice were injected into each hind footpad with a total volume of 100 μl (50 μl per footpad) containing the above-mentioned compounds. Animals were sacrificed 4 days after injection and popliteal lymph nodes were harvested. Lymph nodes were passed through a 70 μm cell strainer and washed twice with DMEM medium (GIBCO BRL) containing 5% fetal calf serum (FCS, SIGMA chemicals). Cells were adjusted to the appropriate cell number in DMEM/5%/FCS. An IFN-γ ELISPOT assay was carried out in triplicates as described (Miyahira et al., 1995). This method is a widely used procedure allowing the quantification of antigen-specific T cells. Lymphocytes were stimulated ex vivo in triplicates with medium (background-control), P1A35-43 peptide, an irrelevant peptide CSP (SYVPSAEQI) pR 60, U-ODN 13 and Concanavalin A (Con A). Spots representing single IFN-γ producing T cells were counted and the number of background spots was substracted from all samples. The high number of spots detected after the stimulation with Con A (data not shown) indicates a good condition of the used lymphocytes. For each experimental group of mice the number of spots/1×10⁶ cells are illustrated in FIG. 13, the standard deviation of ex vivo-stimulated triplicates is given.

This experiment shows that deoxy-Uridine monophosphate modified ODNs induces a strong immune response against the mastocytoma-derived peptide P1A₃₅₋₄₃. This response can be further enhanced by the co-application of poly-L-arginine.

### Example 11

Induction of specific immune responses against a melanoma-derived peptide (TRP-2₁₈₁₋₁₈₈) by a cocktail of deoxy-Uridine monophosphate modified oligonucleotides (U-ODN 15, 20mers).

| | |
|---|---|
| Mice | C57BI/6 (Harlan/Olac) |
| Peptide | TRP-2-peptide (VYDFFVWL), a MHC |
| | class I (H-2Kb)-restricted epi- |
| | tope of mouse tyrosinase related |
| | protein-2 (B16 melanoma, Bloom, |
| | M. B. et al., J Exp. Med 1997, |
| | 185, 453-459), synthesized by |
| | standard solid phase F-moc syn- |
| | thesis, HPLC purified and ana- |
| | lysed by mass spectroscopy for |
| | purity |
| | Dose: 100 μg/mouse |
| Poly-L-arginine 60 (pR60) | Poly-L-arginine with an average |
| | degree of polymerization of 60 |
| | arginine residues; SIGMA chemi- |
| | cals |
| | Dose: 100 μg-0.1 μg/mouse |
| U-ODN 15 | Cocktail of thiophosphate substi- |
| | tuted ODNs containing deoxy- |
| | Uridine monophosphate: |
| | nhh hhh wdu dhh hhh hhh wn, were |
| | synthesized by NAPS GmbH, Göttin- |
| | gen. (n = GCAT, h = CAT, w = AT, |
| | d = GAT) |
| | Dose: 5 nmol-0.005 nmol/mouse |

Experimental groups (4 mice per group)
1. TRP-2₁₈₁₋₁₈₈
2. TRP-2₁₈₁₋₁₈₈+pR60 (100 μg)
3. TRP-2₁₈₁₋₁₈₈+U-ODN 15 (5 nmol)
4. TRP-2₁₈₁₋₁₈₈+U-ODN 15 (0.5 nmol)
5. TRP-2₁₈₁₋₁₈₈+U-ODN 15 (0.05 nmol)
6. TRP-2₁₈₁₋₁₈₈+U-ODN 15 (0.005 nmol)
7. TRP-2₁₈₁₋₁₈₈+pR60 (100 μg)+U-ODN 15 (5 nmol)
8. TRP-2₁₈₁₋₁₈₈+pR60 (10 μg)+U-ODN 15 (0.,5 nmol)
9. TRP-2₁₈₁₋₁₈₈+pR60 (1 μg)+U-ODN 15 (0.05 nmol)
10.TRP-2₁₈₁₋₁₈₈+pR60 (0.1 μg)+U-ODN 15 (0.005 nmol)

On day 0 mice were injected into each hind footpad with a total volume of 100 μl (50 μl per footpad) containing the above-mentioned compounds. Animals were sacrificed 4 days after injection and popliteal lymph nodes were harvested. Lymph nodes were passed through a 70 μm cell strainer and washed twice with DMEM medium (GIBCO BRL) containing 5% fetal calf serum (FCS, SIGMA chemicals). Cells were adjusted to the appropriate cell number in DMEM/5%/FCS. An IFN-γ ELISPOT assay was carried out in triplicates as described (Miyahira et al., 1995). This method is a widely used procedure allowing the quantification of antigen-specific T cells. Lymphocytes were stimulated ex vivo in triplicates with medium (background-control), TRP-2₁₈₁₋₁₈₈-peptide, an irrelevant peptide OVA₂₅₇₋₂₆₄ pR 60, U-ODN15 and Concanavalin A (Con A). Spots representing single IFN-γ producing T cells were counted and the number of background spots was substracted from all samples. The high number of spots detected after the stimulation with Con A (data not shown) indicates a good condition of the used lymphocytes. For each experimental group of mice the number of IFN-γ-producing cells/1×10⁶ cells are illustrated in FIG. 14, the standard deviation of ex vivo-stimulated triplicates is given.

This experiment shows that the injection of TRP-2₁₈₁₋₁₈₈ (hydrophobic peptide) with a cocktail of deoxy-Uridine monophosphate modified ODNs (20mers, 5nmol) strongly enhances TRP-2₁₈₁₋₁₈₈-specific immune responses compared to the injection of TRP-2₁₈₁₋₁₈₈ alone. Even when 10 times less of the U-ODN 15 was used (0.5 nmol) a strong immune response could be induced. The co-injection of poly-L-arginine with peptide and U-ODN 15 (5nmol) does not influence this strong response.

### Example 12

Induction of specific immune responses against a melanoma-derived peptide (TRP-2₁₈₁₋₁₈₈) by a cocktail of deoxy-Uridine monophosphate modified oligonucleotides (U-ODN 16, 10mers).

| | |
|---|---|
| Mice | C57BI/6 (Harlan/Olac) |
| Peptide | TRP-2-peptide (VYDFFVWL), a MHC |
| | class I (H-2Kb)-restricted epi- |
| | tope of mouse tyrosinase related |
| | protein-2 (B16 melanoma, Bloom, |
| | M. B. et al., J Exp. Med 1997, |
| | 185, 453-459), synthesized by |
| | standard solid phase F-moc syn- |
| | thesis, HPLC purified and ana- |
| | lysed by mass spectroscopy for |
| | purity |
| | Dose: 100 μg/mouse |
| U-ODN 16 | Cocktail of thiophosphate substi- |
| | tuted ODNs containing deoxy- |
| | Uridine monophosphate: |
| | hhh wdu dhh h, were synthesized |
| | by NAPS GmbH, Göttingen. (n = |
| | GCAT, h = CAT, w = AT, d = GAT) |
| | Dose: 10 nmol/mouse |
| ODN 20 | Cocktail of thiophosphate substi- |
| | tuted ODNs: |
| | hhh wdd dhh h, were synthesized |
| | by NAPS GmbH, Göttingen. (n = |
| | GCAT, h = CAT, w = AT, d = GAT) |
| | Dose: 10 nmol/mouse |

Experimental groups (4 mice per group)
1. TRP-2₁₈₁₋₁₈₈
2. TRP-2₁₈₁₋₁₈₈+U-ODN 16 (10 nmol)
3. TRP-2₁₈₁₋₁₈₈+ODN 20 (10 nmol)

On day 0 mice were injected into each hind footpad with a total volume of 100 μl (50 μl per footpad) containing the above-mentioned compounds. Animals were sacrificed 4 days after injection and popliteal lymph nodes were harvested. Lymph nodes were passed through a 70 μm cell strainer and washed twice with DMEM medium (GIBCO BRL) containing 5% fetal calf serum (FCS, SIGMA chemicals). Cells were adjusted to the appropriate cell number in DMEM/5%/FCS. An IFN-γ ELISPOT assay was carried out in triplicates as described (Miyahira et al., 1995). This method is a widely used procedure allowing the quantification of antigen-specific T cells. Lymphocytes were stimulated ex vivo in triplicates with medium (background-control), TRP-2₁₈₁₋₁₈₈-peptide, an irrelevant peptide OVA_{256-264,} U-ODN 16, ODN 20 and Concanavalin A (Con A). Spots representing single IFN-γ producing T cells were counted and the number of background spots was substracted from all samples. The high number of spots detected after the stimulation with Con A (data not shown) indicates a good condition of the used lymphocytes. For each experimental group of mice the number of IFN-γ-producing cells/1×10⁶ cells are illustrated in FIG. 15, the standard deviation of ex vivo-stimulated triplicates is given.

This experiment shows that the injection of TRP-2₁₈₁₋₁₈₈ (hydrophobic peptide) with a cocktail of deoxy-Uridine monophosphate modified ODNs (lOmers) strongly enhances TRP-2₁₈₁₋₁₈₈-specific immune responses compared to the injection of TRP-2₁₈₁₋₁₈₈ alone or in combination with ODN 20.

### Example 13

Activation of human PBMC

| | |
|---|---|
| Cells | human PBMC isolated from buffy coats |
| CpG-ODN 2006 | thiophosphate substituted ODNs containing CpG |
| | motifs: 5′-tcg tcg ttt tgt cgt ttt gtc gtt-3′ |
| | were synthesized by Purimex Nucleic Acids |
| | Technology, Göttingen |
| | Concentration: 1 μM |
| I-ODN 2b | ODNs containing deoxyinosine: 5′ tcc atg aci |
| | ttc ctg atg ct 3′ were synthesized by |
| | Purimex Nucleic Acids Technology, |
| | Göttingen |
| | Concentration: 1 μM |
| o-d(IC)₁₃ | oligo-d(IC)₁₃ (5′ ICI CIC ICI CIC ICI CIC ICI |
| | CIC IC 3′, DNA) was synthesized by Purimex |
| | Nucleic Acids Technology, Göttingen |
| | Concentration: 1 μM |
| KLK | KLKLLLLLKLK-COOH was synthesized by MPS |
| | (Multiple Peptide System, USA) |
| | Concentration: 16.8 μg/ml |
| Poly-L-arginine 60 (pR60) | poly-L-arginine with an average degree |
| | of polymerization of 60 arginine residues |
| | (by viscosity); Sigma |
| | Concentration: 10 μg/ml |

Human PBMC were isolated from a buffy coat via Ficoll (PAA, Austria) and stimulated as followed (2×10⁶/ml/well, 24-well-plate):

| | | |
|---|---|---|
| 1. | medium | |
| 2. | CpG-ODN 2006 | 1 μM |
| 3. | I-ODN 2b | 1 μM |
| 4. | o-d(IC)₁₃ | 1 μM |
| 5. | pR 60 | 10 μg/ml |
| 6. | KLK | 16.8 μg/ml |
| 7. | I-ODN 2b + pR 60 | 1 μM + 10 μg/ml |
| 8. | o-d(IC)₁₃ + pR 60 | 1 μM + 10 μg/ml |
| 9. | I-ODN 2b + KLK | 1 μM + 16.8 μg/ml |
| 10. | o-d(IC)₁₃ + KLK | 1 μM + 16.8 μg/ml |

After 18 h of incubation, the cells were analyzed by FACS for the expression of HLA-DR and the co-stimulatory molecules CD40, CD80, CD86. FIG. 13 shows histogram overlays of single stained PBMCs (gated on living cells in FSC:SSC dot plot). Each single graphic contains results obtained upon incubation with medium (negative control) and CpG-ODN 2006 for comparison purposes. Poly-L-arginine and KLK upregulate the expression of CD40, CD80 and CD86, poly-L-arginine has no effect on HLA-DR expression, whereas KLK decrease its expression. I-ODN 2b and o-d(IC)₁₃ strongly increase the expression of CD40 and CD86, but have no effect on the expression of CD86 and HLA-DR. However, all combinations (I-ODN 2b/pR, I-ODN 2b/KLK, o-d(IC)₁₃/pR and o-d(IC)₁₃/KLK) strongly increase the expression of all analyzed surface molecules (HLA and co-stimulatory molecules) indicating the activation of antigen presenting cells among PBMCs.

### Example 14

Activation of human myeloid dendritic cells

| | |
|---|---|
| Cells | human myeloid dendritic cells generated from |
| | leucopheresates |
| CpG-ODN | thiophosphate substituted ODNs containing CpG |
| 2006 | motifs: 5′-tcg tcg ttt tgt cgt ttt gtc gtt-3′ |
| | were synthesized by Purimex Nucleic Acids Technology, |
| | Göttingen |
| | Concentration: 1 μM |
| I-ODN 2b | ODNs containing deoxyinosine: 5′ tcc atg aci ttc |
| | ctg atg ct 3′ were synthesized by Purimex Nucleic |
| | Acids Technology, Göttingen |
| | Concentration: 1 μM |
| o-d(IC)₁₃ | oligo-d(IC)₁₃ (5′ ICI CIC ICI CIC ICI CIC ICI |
| | CIC IC 3′, DNA) was synthesized by Purimex |
| | Nucleic Acids Technology, Göttingen |
| | Concentration: 1 μM |
| KLK | KLKLLLLLKLK-COOH was synthesized by MPS (Multiple |
| | Peptide System, USA) |
| | Concentration: 16.8 μg/ml |
| poly (I:C) | Polyinosinic-polycytidylic acid is a synthetic |
| | double stranded RNA molecule which |
| | was purchased from Amersham |
| | Concentration: 10 μg/ml |

On day 0 frozen human leucopheresates from 2 different donors (HHE and PHÖ) were thawed, the cells were transferred into HBSS-buffer (Bio Whittaker Europe) and centrifuged (411× g, 4° C., 7 min). The resulting cell pellet of each donor was resuspended in RPMI 1640 (Bio Whittaker Europe) supplemented with 1,5% autologous plasma and the cell-suspensions were seeded (2×10⁷/2 ml/well) in 6-well plates (COSTAR). After an incubation time of 50-60 min at 37° C./5%CO₂, non adherent cells were rinsed off, adherent cells were washed with lXPBS (PAA) and further incubated at 37° C./5%CO₂ with 3 ml/well X-VIVO (Bio Whittaker Europe) supplemented with 1,5% autologous plasma, 800 U/ml GM-CSF (Novartis, LEUKOMAX) and 100 U/ml IL-4 (Strathmann Biotech GmbH). On day 2, 1 ml supernatant was exchanged by 1 ml X-VIVO+1,5% autologous plasma+100 U/ml IL-4+1600 U/ml GM-CSF. On day 5, non-adherent cells of each donor were harvested, counted and about 1,2-1,5×10⁶ cells/3 ml X-VIVO/1,5% autologous plasma were seeded per well in 6-well plates (COSTAR). The obtained myeloid dendritic cells were stimulated as followed:

| | | |
|---|---|---|
| 1. | Medium | |
| 2. | poly (I:C) | 10 μg/ml |
| 3. | CpG-ODN 2006 | 1 μM |
| 4. | I-ODN 2b | 1 μM |
| 5. | o-d(IC)₁₃ | 1 μM |
| 6. | KLK | 16.8 μg/ml |
| 7. | KLK + CpG 2006 | 16.8 μg/ml + 1 μM |
| 8. | KLK + I-ODN 2b | 16.8 μg/ml + 1 μM |
| 9. | KLK + o-d(IC)₁₃ | 16.8 μg/ml + 1 μM |

After 24 hours of incubation (37° C./5%CO₂), cells were harvested and double stainings against HLA-DR versus the co-stimulatory molecules CD80, CD86, CD40, and the maturation marker CD83, as well as CD1a versus CD80 were performed and analyzed by FACS. Table 1 shows the percentage of double positive cells (gated on living cells in the FSC:SSC dotplot) stained for the different cell-surface molecules as indicated.

TABLE 1
Activation of human myeloid dendritic cells

| | HLA-DR/CD80 | | HLA-DR/CD86 | | HLA-DR/CD83 | | HLA-DR/CD40 | | CD1a/CD80 | |
|---|---|---|---|---|---|---|---|---|---|---|
| | (%) | | (%) | | (%) | | (%) | | (%) | |
| donor: | HHE | PHÖ | HHE | PHÖ | HHE | PHÖ | HHE | PHÖ | HHE | PHÖ |
| Medium | 1.12** | 1.42 | 71.91 | 54.66 | 0.77 | 0.84 | — | 2.58 | 0.90 | 1.34 |
| pIC* | 20.37 | 12.71 | 69.03 | 52.57 | 5.33 | 3.45 | — | 15.84 | 1.20 | 1.47 |
| CpG-ODN 2006 | 1.20 | 1.85 | 71.03 | 47.04 | 0.70 | 1.23 | — | 6.06 | 0.60 | 1.59 |
| I-ODN 2b | 1.25 | 1.30 | 65.29 | 49.94 | 0.68 | 0.66 | — | 4.89 | 0.80 | 1.03 |
| o-d(IC)₁₃ | 1.27 | 1.25 | 66.44 | 49.99 | 0.50 | 0.62 | — | 7.09 | 0.80 | 1.23 |
| KLK | 5.13 | 4.91 | 80.50 | 60.24 | 3.87 | 4.71 | — | 11.07 | 9.70 | 6.94 |
| KLK + I-ODN 2b | 20.33 | 14.22 | 75.20 | 66.70 | 20.79 | 13.88 | — | 27.66 | 30.70 | 24.32 |
| KLK + o-d(IC)₁₃ | 16.77 | 13.52 | 75.46 | 65.85 | 14.61 | 13.02 | — | 24.43 | 25.00 | 21.30 |

*pIC [10 μg/ml], CpG-ODN 2006 [1 μM], I-ODN 2b [1 μM], o-d(IC)₁₃ [1 μM], KLK [16.8 μg/ml]
**percentage of double positive cells; total living cells = 100%

Compared to the medium stimulation, which represents the negative control in this experiment, poly (IC) as positive control increases the number of HLA-DR/CD80, HLA-DR/CD83, HLA-DR/CD40 and CDla/CD80 positive cells. The incubation of myeloid dendritic cells with I-ODN 2b or o-d(IC)₁₃ resulted in no remarkable in-crease in the expression of the analyzed cell-surface molecules, whereas upon stimulation with KLK an activation at low level is observable. However, the stimulation of human myeloid dendritic cells with the combinations of KLK/I-ODN2b or KLK/o-d(IC)₁₃ strongly increases the number of HLA-DR/CD80, HLA-DR/CD86, HLA-DR/CD83, HLA-DR/CD40 and CDla/CD80 positive cells. The high expression of the analyzed molecules indicates a status of maturation and activation of these antigen-presenting cells, which implies their potential to stimulate efficiently T cells.

Other preferred sequences according to the present invention are:

Sequences useful for stimulating natural killer cell (NK) lytic acitivity in a subject such as a human. Specific, but non-limiting examples of such sequences include:

TC(dI/dU)TC(dI/dU)TTGTC(dI/dU)TTGTC(dI/dU)TT,
TC(dI/dU)TC(dI/dU)TTTTGTC(dI/dU)TTTTGTC(dI/dU)TT,
TC(dI/dU)TC(dI/dU)TTGTC(dI/dU)TTTTGTC(dI/dU)TT,
GC(dI/dU)TGC(dI/dU)TTGTC(dI/dU)TTGTC(dI/dU)TT,
TGTC(dI/dU)TTTGTC(dI/dU)TTTGTC(dI/dU)TT,
TGTC(dI/dU)TTGTC(dI/dU)TTGTC(dI/dU)TT and
TC(dI/dU)TC(dI/dU)TC(dI/dU)TC(dI/dU)TT.

Sequences useful for stimulating B cell proliferation in a subject such as a human. Specific, but non-limiting examples of such sequences include:

TCCTGTC(dI/dU)TTCCTTGTC(dI/dU)TT,
TCCTGTC(dI/dU)TTTTTTGTC(dI/dU)TT,
TC(dI/dU)TC(dI/dU)CTGTCTGCCCTTCTT,
TC(dI/dU)TC(dI/dU)CTGTTGTC(dI/dU)TTTCTT,
TC(dI/dU)TC(dI/dU)TTTTGTC(dI/dU)TTTTGTC(dI/dU)TT
TC(dI/dU)TC(dI/dU)TTGTC(dI/dU)TTTTGTC(dI/dU)TT and
TGTC(dI/dU)TTGTC(dI/dU)TTGTC(dI/dU)TT.

Sequences useful as an adjuvant for use during antibody production in a mammal. Specific, but non-limiting examples of such sequences include:

TCCATGAC(dI/dU)TTCCTGAC(dI/dU)TT,
GTC(dI/dU)(T/C)T and TGTC(dI/dU)(T/C)T.

Sequences for treating or preventing the symptoms of an asthmatic disorder by redirecting a subject's immune response from Th2 to Th1. An exemplary sequence includes TCCATGAC (dI/dU) TTCCTGAC (dI/dU) TT.

ODN induction of NK Lvtic Activity (LU)

ACCATGGAC(dI/dU)ATCTGTTTCCCCTC
TCTCCCAGC(dI/dU)TGC(dI/dU)CCAT
TACC(dI/dU)C(dI/dU)TGC(dI/dU)ACCCTCT
ACCATGGAC(dI/dU)AACTGTTTCCCCTC
ACCATGGAC(dI/dU)AGCTGTTTCCCCTC
ACCATGGAC(dI/dU)ACCTGTTTCCCCTC
ACCATGGAC(dI/dU)TACTGTTTCCCCTC
ACCATGGAC(dI/dU)GTCTGTTTCCCCTC
ACCATGGAC(dI/dU)TTCTGTTTCCCCTC
GCATGAC(dI/dU)TTGAGCT
CAC(dI/dU)TTGAGGGGCAT
CTGCTGAGACTGGAG
TCAGC(dI/dU)TGC(dI/dU)CC
ATGAC(dI/dU)TTCCTGAC(dI/dU)TT
TCTCCCAGC(dI/dU)GGC(dI/dU)CAT
TCTCCCAGC(dI/dU)C(dI/dU)C(dI/dU)CCAT
TCCATGTC(dI/dU)TTCCTGTC(dI/dU)TT
TCCATAGC(dI/dU)TTCCTAGC(dI/dU)TT
TC(dI/dU)TC(dI/dU)CTGTCTCC(dI/dU)CTTCTT
TCCTGAC(dI/dU)TTCCTGAC(dI/dU)TT

Induction of NK LU bv Phosphorothioate CpdI/dU ODN with Good Motifs

TCCATGTC(dI/dU)TTCCTGTC(dI/dU)TT
TCCTGTC(dI/dU)TTCCTGTC(dI/dU)TT
TCCATGTC(dI/dU)TTTTTGTC(dI/dU)TT
TCCTGTC(dI/dU)TTCCTTGTC(dI/dU)TT
TCCTTGTC(dI/dU)TTCCTGTC(dI/dU)TT
TCCTGTC(dI/dU)TTTTTTGTC(dI/dU)TT
TC(dI/dU)TC(dI/dU)CTGTCTCC(dI/dU)CTTCTT
TC(dI/dU)TC(dI/dU)CTGTCTGCCCTTCTT
TC(dI/dU)TC(dI/dU)CTGTTGTC(dI/dU)TTTCTT
TCCATGTZGTTCCTGTZGTT
TCCAGGACTTCTCTCAGGTT
TCCATGC(dI/dU)TGC(dI/dU)TGC(dI/dU)TTTT
TCCATGC(dI/dU)TTGC(dI/dU)TTGC(dI/dU)TT
TCCAC(dI/dU)AC(dI/dU)TTTTC(dI/dU)AC(dI/dU)TT
TC(dI/dU)TC(dI/dU)TTGTC(dI/dU)TTGTC(dI/dU)TT
TC(dI/dU)TC(dI/dU)TTTTGTC(dI/dU)TTTTGTC(dI/dU)TT
NTC(dI/dU)TC(dI/dU)TTGTC(dI/dU)TTTTGTC(dI/dU)TT
GC(dI/dU)TGC(dI/dU)TTGTC(dI/dU)TTGTC(dI/dU)TT
GC(dI/dU)GC(dI/dU)GGC(dI/dU)GC(dI/dU)C(dI/dU)C(dI/dU)CCC
TGTC(dI/dU)TTTGTC(dI/dU)TTTGTC(dI/dU)TT
TGTC(dI/dU)TTGTC(dI/dU)TTGTC(dI/dU)TTGTC(dI/dU)TT
TGTC(dI/dU)TTGTC(dI/dU)TTGTC(dI/dU)TT
TC(dI/dU)TC(dI/dU)TC(dI/dU)TC(dI/dU)TT
TGTC(dI/dU)TTGTC(dI/dU)TT

Induction of human B cell proliferation bv Phosphorothioate CpDI/dU ODN

| | |
|---|---|
| 1840 | TCCATGTC(dI/dU)TTCCTGTC(dI/dU)TT |
| 1841 | TCCATAGC(dI/dU)TTCCTAGC(dI/dU)TT |
| 1960 | TCCTCTC(dI/dU)TTCCTGTC(dI/dU)TT |
| 1961 | TCCATGTC(dI/dU)TTTTTGTC(dI/dU)TT |
| 1962 | TCCTGTC(dI/dU)TTCCTTGTC(dI/dU)TT |
| 1963 | TCCTTGTC(dI/dU)TTCCTGTC(dI/dU)TT |
| 1965 | TCCTGTC(dI/dU)TTTTTTGTC(dI/dU)TT |
| 1967 | TC(dI/dU)TC(dI/dU)CTGTCTGCCCTTCTT |
| 1968 | TC(dI/dU)TC(dI/dU)CTGTTGTC(dI/dU)TTTCTT |
| 1982 | TCCAGGACTTCTCTCAGGTT |
| 2002 | TCCAAC(dI/dU)TTITC(3AC(dI/dU)TT |
| 2005 | TC(dI/dU)TC(dI/dU)TTGTC(dI/dU)TTGTC(dI/dU)TT |
| 2006 | T˜TC(dI/dU)TTTTGTC(dI/dU)TTTTGTC(dI/dU)TT |
| 2007 | TC(dI/dU)TC(dI/dU)TTGTC(dI/dU)TTTTGTC(dI/dU)TT |
| 2008 | GC(dI/dU)TGC(dI/dU)TTGTC(dI/dU)TTGTTT |
| 2010 | GC(dI/dU)GC(dI/dU)GGC(dI/dU)GC(dI/dU)C(dI/dU)C(dI/dU)CCC |
| 2012 | TGTC(dI/dU)TTTGTC(dI/dU)TTTGTC(dI/dU)TT |
| 2013 | TGTC(dI/dU)TTGT˜TTGT˜TTGT˜TT |
| 2014 | TGTC(dI/dU)TTGTC(dI/dU)TTGTC(dI/dU)TT |
| 2015 | TC(dI/dU)TC(dI/dU)TC(dI/dU)TC(dI/dU) |
| 2016 | TGTC(dI/dU)TTGTC(dI/dU)TT |

Induction of human IL-12 secretion bv Phosphorothioate CpdI/dU ODN

| | |
|---|---|
| 1962 | TCCTGTC(dI/dU)TTCCTTGTC(dI/dU)TT |
| 1965 | TCCTGTC(dI/dU)TTTTTTGTC(dI/dU)TT |
| 1967 | TC(dI/dU)TC(dI/dU)CTGTCTGCCCTTCTT |
| 1968 | TC(dI/dU)TC(dI/dU)CTGTTGTC(dI/dU)TTTCTT |
| 2005 | TC(dI/dU)TC(dI/dU)TTGTC(dI/dU)TTGTC(dI/dU)TT |
| 2006 | TC(dI/dU)TC(dI/dU)TTTTGTC(dI/dU)TTTTGTC(dI/dU)TT |
| 2014 | TGTC(dI/dU)TTGTC(dI/dU)TTGTC(dI/dU)TT |
| 2015 | TC(dI/dU)TC(dI/dU)TC(dI/dU)TC(dI/dU)TT |
| 2016 | TGTC(dI/dU)TTGTC(dI/dU)TT |

Different CpdI/dU motifs stimulate optimal murine B cell and NK activation

| | |
|---|---|
| 1668 | TCCATGAC(dI/dU)TTCCTGATGCT |
| 1758 | TCTCCCAGC(dI/dU)TGC(dI/dU)CCAT |

CpDI/dU ODN for stimulating natural killer-cell (NK) lytic activity in a subject such as a human. Specific, but nonlimiting examples of such sequences include:

TC(dI/dU)TC(dI/dU)TTGTC(dI/dU)TTGTC(dI/dU)TT,
TC(dI/dU)TC(dI/dU)TTTTGTC(dI/dU)TTTTGTC(dI/dU)TT,
TC(dI/dU)TC(dI/dU)TTGTC(dI/dU)TTTTGTC(dI/dU)TT,
GC(dI/dU)TGC(dI/dU)TTGTC(dI/dU)TTGTC(dI/dU)TT,
TGTC(dI/dU)TTTGTC(dI/dU)TTTGTC(dI/dU)TT,
TGTC(dI/dU)TTGTC(dI/dU)TTGTC(dI/dU)TT, and
TC(dI/dU)TC(dI/dU)TC(dI/dU)TC(dI/dU)TT.

Sequences useful for stimulating B cell proliferation. Specific, but nonlimiting examples of such sequences include:

TCCTGTC(dI/dU)TTCCTTGTC(dI/dU)TT,
TCCTGTC(dl/dU)TTTTTTGTC(dI/dU)TT,
TC(dI/dU)TC(dI/dU)CTGTCTGCCCTTCTT,
TC(dI/dU)TC(dI/dU)CTGTTGTC(dI/dU)TTTCTT,
TC(dI/dU)TC(dI/dU)TTTTGTC(dI/dU)TTTTGTC(dI/dU)TT,
TC(dI/dU)TC(dI/dU)TTGTC(dI/dU)TTTTGTC(dI/dU)TT and
TGTC(dI/dU)TTGTC(dI/dU)TTGTC(dI/dU)TT.

Exemplary sequences include

| | |
|---|---|
| TCCATGTC(dI/dU)CTCCTGATGCT, | (SEQ ID NO:102) |
| TCCATGTC(dI/dU)TTCCTGATGCT, | |
| TC(dI/dU)TC(dI/dU)TTTTGTC(dI/dU)TTTTGTC(dI/dU)TT, | |
| TC(dI/dU)TC(dI/dU)TTGTC(dI/dU)TTGTC(dI/dU)TT; | |
| TC(dI/dU)TC(dI/dU)TTTTGTC(dI/dU)TTTTGTC(dI/dU)TT, | |
| TC(dI/dU)TC(dI/dU)TTGTC(dI/dU)TTTTGTC(dI/dU)TT, | |
| GC(dI/dU)TGC(dI/dU)TTGTC(dI/dU)TTGTC(dI/dU)TT, | |
| TGTC(dI/dU)TTTGTC(dI/dU)TTTGTC(dI/dU)TT, | |
| TGTC(dI/dU)TTGTC(dI/dU)TTGTC(dI/dU)TT, | |
| TC(dI/dU)TC(dI/dU)TC(dI/dU)TC(dI/dU)TT, | |
| TCCTGTC(dI/dU)TTCCTTGTC(dI/dU)TT, | |
| TCCTGTC(dI/dU)TTTTTTGTC(dI/dU)TT, | |
| TC(dI/dU)TC(dI/dU)CTGTCTGCCCTTCTT, | |
| TC(dI/dU)TC(dI/dU)CTGTTGTC(dI/dU)TTTCTT, | |
| TC(dI/dU)TC(dI/dU)TTTTGTC(dI/dU)TTTTGTC(dI/dU)TT, | |
| TC(dI/dU)TC(dI/dU)TTGTC(dI/dU)TTTTGTC(dI/dU)TT | |
| TGTC(dI/dU)TTGTC(dI/dU)TTGTC(dI/dU)TT, | |
| TCCATGAC(dI/dU)TTCCTGAC(dI/dU)TT, | |
| GTC(dI/dU)(T/C)T and | |
| TGTC(dI/dU)(T/C)T. | |

TABLE 1
sequences

GCTAGAC(dI/dU)TTAGC(dI/dU)T
GCTAGATGTTAGC(dI/dU)T
GCTAGAC(dI/dU)TTAGZGT GCATGAC(dI/dU)TTGAGCT
ATGGAAGGTCCAGC(dI/dU)TTCTC
ATC(dI/dU)ACTCTC(dI/dU)AGC(dI/dU)TTCTC
ATZGACTCTC(dI/dU)AGC(dI/dU)TTCTC
ATC(dI/dU)ACTCTC(dI/dU)AGC(dI/dU)TTZTC
ATC(dI/dU)ACTCTC(dI/dU)AAC(dI/dU)TTCTC
GAGAAC(dI/dU)CTGGACCTTCCAT
GAGAAC(dI/dU)CTC(dI/dU)ACCTTCCAT
GAGAAC(dI/dU)CTC(dI/dU)ACCTTC(dI/dU)AT
GAGCAAGCTGGACCTTCCAT
GAGAAC(dI/dU)CTGGACZTTCCAT
GAGAAC(dI/dU)ATGGACCTTCCAT
GAGAAC(dI/dU)CTCCAGCACTGAT
CCATGTC(dI/dU)GTCCTGATGCT
TCCATGTC(dI/dU)GTZCTGATGCT
TCCATGAC(dI/dU)TTCCTGATGCT
TCCATGTC(dI/dU)GTCCTGAC(dI/dU)CA
TCAAC(dI/dU)TT
TCAGC(dI/dU)CT
TCTTC(dI/dU)AT
TCTTC(dI/dU)AA
CAAC(dI/dU)TT
CCAAC(dI/dU)TT
CAAC(dI/dU)TTCT
TCAAC(dI/dU)TC
ATGGACTCTCCAGC(dI/dU)TTCTC
ATAGGAGGTCCAAC(dI/dU)TTCTC
ATC(dI/dU)ACTCTC(dI/dU)AGC(dI/dU)TTCTC
ATGGAGGCTCCATC(dI/dU)TTCTC
ATC(dI/dU)ACTCTC(dI/dU)AGZGTTCTC
GCATGAC(dI/dU)TTGAGCT
TCCATGTC(dI/dU)GTCCTGATGCT
TCCATGGC(dI/dU)GTCCTGATGCT
TCCATGAC(dI/dU)GTCCTGATGCT
TCCATGTC(dI/dU)ATCCTGATGCT
TCCATGTC(dI/dU)CTCCTGATGCT
TCCATGTC(dI/dU)TTCCTGATGCT
TCCATAAC(dI/dU)TTCCTGATGCT
TCCATGAC(dI/dU)TCCCTGATGCT
TCCATCAC(dI/dU)TGCCTGATGCT
GGGGTCAAC(dI/dU)TTGAC(dI/dU)GGG
GGGGTCAGTC(dI/dU)TGAC(dI/dU)GGG
GCTAGAC(dI/dU)TTAGTGT
TCCATGTC(dI/dU)TTCCTGATGCT
ACCATGGAC(dI/dU)ATCTGTTTCCCCTC
TCTCCCAGC(dI/dU)TGC(dI/dU)CCAT
TACC(dI/dU)C(dI/dU)TGC(dI/dU)ACCCTCT
ACCATGGAC(dI/dU)AACTGTTTCCCCTC
ACCATGGAC(dI/dU)AGCTGTTTCCCCTC
ACCATGGAC(dI/dU)ACCTGTTTCCCCTC
ACCATGGAC(dI/dU)TACTGTTTCCCCTC
ACCATGGAC(dI/dU)GTCTGTTTCCCCTC
ACCATGGAC(dI/dU)TTCTGTTTCCCCTC
CAC(dI/dU)TTGAGGGGCAT
TCAGC(dI/dU)TGC(dI/dU)CC
ATGAC(dI/dU)TTCCTGAC(dI/dU)TT
TCTCCCAGC(dI/dU)GGC(dI/dU)CAT
TCTCCCAGC(dI/dU)C(dI/dU)C(dI/dU)CCAT
TCCATGTC(dI/dU)TTCCTGTC(dI/dU)TT
TCCATAGC(dI/dU)TTCCTAGC(dI/dU)TT
TC(dI/dU)TC(dI/dU)CTGTCTCC(dI/dU)CTTCTT
TCCTGAC(dI/dU)TTCCTGAC(dI/dU)TT
TCCTGTC(dI/dU)TTCCTGTC(dI/dU)TT
TCCATGTC(dI/dU)TTTTTGTC(dI/dU)TT
TCCTGTC(dI/dU)TTCCTTGTC(dI/dU)TT
TCCTTGTC(dI/dU)TTCCTGTC(dI/dU)TT
TCCTGTC(dI/dU)TTTTTTGTC(dI/dU)TT
TC(dI/dU)TC(dI/dU)CTGTCTGCCCTTCTT
TC(dI/dU)TC(dI/dU)CTGTTGTC(dI/dU)TTTCTT
TCCATGC(dI/dU)TGC(dI/dU)TGC(dI/dU)TTTT
TCCATGC(dI/dU)TTGC(dI/dU)TTGC(dI/dU)TT
TCCAC(dI/dU)AC(dI/dU)TTTTC(dI/dU)AC(dI/dU)TT
TC(dI/dU)TC(dI/dU)TTGTC(dI/dU)TTGTC(dI/dU)TT
TC(dI/dU)TC(dI/dU)TTTTGTC(dI/dU)TTTTGTC(dI/dU)TT
TC(dI/dU)TC(dI/dU)TTGTC(dI/dU)TTTTGTC(dI/dU)TT
GC(dI/dU)TGC(dI/dU)TTGTC(dI/dU)TTGTC(dI/dU)TT
TGTC(dI/dU)TTTGTC(dI/dU)TTTGTC(dI/dU)TT
TGTC(dI/dU)TTGTC(dI/dU)TTGTC(dI/dU)TTGTC(dI/dU)TT
TGTC(dI/dU)TTGTC(dI/dU)TTGTC(dI/dU)TT
TC(dI/dU)TC(dI/dU)TC(dI/dU)TC(dI/dU)TT
TGTC(dI/dU)TTGTC(dI/dU)TT
TCCATAGC(dI/dU)TTCCTAGC(dI/dU)TT
TCCATGAC(dI/dU)TTCCTGAC(dI/dU)TT
GTC(dI/dU)TT
TGTC(dI/dU)TT
TCTCCCAGC(dI/dU)TGC(dI/dU)CCAT
GTC(dI/dU)CT
TGTC(dI/dU)CT

1-48. (canceled)

49. A method of stimulating an immune system comprising:
obtaining a pharmaceutical composition comprising an oligodeoxynucleic acid molecule (ODN) having a structure of formula (I): 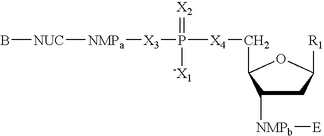
wherein:
R1 is selected from hypoxanthine and uracile,
any X is O or S,
any NMP is a 2′ deoxynucleoside monophosphate or monothiophosphate, further defined as deoxyadenosine-, deoxyguanosine-, deoxyinosine-, deoxycytosine-, deoxyuridine-, deoxythymidine-, 2-methyldeoxyinosine-, 5-methyl-deoxycytosine-, deoxypseudouridine-, deoxyribosepurine-, 2-amino-deoxyribosepurine-, 6-S-deoxyguanine-, 2-dimethyl-deoxyguanosine- or N-isopentenyl-deoxyadenosinemonophosphate or -monothiophosphate;
NUC is a 2′ deoxynucleoside further defined as deoxyadenosine-, deoxyguanosine-, deoxyinosine-, deoxycytosine-, deoxyinosine-, deoxythyrnidine-, 2-methyl-deoxyuridine-, 5-methyl-deoxycytosine-, deoxypseudouridine-, deoxyribosepurine-, 2-amino-deoxyribosepurine- , 6-S-deoxyguanine-, 2-dimethyl-deoxyguanosine- or N-isopentenyldeoxyadenosine;
a and b are integers from 0 to 100 with the proviso that a+b is between 4 and 150;
B and E are common groups for 5′ or 3′ ends of nucleic acid molecules; and administering the pharmaceutical composition to a subject.

50. The method of claim 49, further defined as a method of immunostimulating the subject without vaccinating the subject.

51. The method of claim 49, wherein any NMP is further defined as deoxyadenosine-, deoxyguanosine-, deoxyinosine-, deoxycytosine-, deoxyuridine-, deoxythymidine-, 2-methyl-deoxyuridine-, 5-methyl-deoxycytosine-monophosphate or -monothiophosphate.

52. The method of claim 49, wherein a+b is between 10 and 60.

53. The method of claim 52, wherein a+b is between 15 and 40.

54. The method of claim 49, wherein at least one of X₁ and X₂ is S and at least one of X₃ and X₄ is O.

55. The method of claim 54, wherein any NMP is a nucleosidemonothiophosphate.

56. The method of claim 49, wherein B and E are independently —H, —CH₃, —COH, —COCH₃, —OH, —CHO, —PO₄, PS₂O₂, —PSO₃, —PS₄, —PS₄, —SO₃, —PO₄—(CH₂)₁₋₆—NH , or —PO₄—(CH₂)₁₋₆—NH-Label.

57. The method of claim 49, wherein said ODN contains at least one 2′deoxycytosine-monophosphate or -monothiophosphate 3′-adjacent to a 2′-deoxyuridine-monophosphate or -monothiophosphate.

58. The method of claim 49, wherein said ODN comprises the sequence wdu, wherein:
u is deoxyuridine-monophosphate or -monothiophosphate;
w is a 2′-deoxynucleoside monophosphate or monothiophosphate, further defined as deoxyadenosine- or deoxythymidine-monophosphate or -monothiophosphate; and
d is a 2′-deoxynucleoside monophosphate or monothiophosphate, further defined as deoxyadenosine-, deoxyguanosine- or deoxythymidine-monophosphate or -monothiophosphate.

59. The method of claim 49, wherein said ODN comprises the sequence wdi, wherein:
i is deoxyinosine-monophosphate or -monothiophosphate;
w is a 2′-deoxynucleoside monophosphate or monothiophosphate, further defined as deoxyadenosine- or deoxythymidine-monophosphate or -monothiophosphate; and
d is a 2′-deoxynucleoside monophosphate or monothiophosphate, further defined as deoxyadenosine-, deoxyguanosine- or deoxythymidine-monophosphate or -monothiophosphate.

60. The method of claim 49, wherein said ODN contains at least one 2′deoxycytosine-monophosphate or -monothiophosphate 3′-adjacent to a 2′-deoxyinosine-monophosphate or -monothiophosphate.

61. The method of claim 49, wherein the ODN contains at least one structure represented by the following general formula:
5′-NMPn . . . NMP3NMP2NMP1NMP1′NMP2′NMP3′. . . NMPn′-3′ (II)
wherein n is an integer from 3 to 50; NMP1, NMP2, NMP3, . . . , NMPn and NMP1′, NMP2′, NMP3′, . . . , NMPn′ are each a monodeoxyribonucleotide; NMP1, NMP2, NMP3, . . . and Xn may be the same or different nucleotides, wherein at least one of said monodeoxyribonucleotides is dl or dU; and bases in NMP1 and NMP1′, in NMP2 and NMP2′, in NMP3 and NMP3′, in . . . , and in NMPn and NMPn′ are, except dI or dU residues, complementary with each other as defined by Watson & Crick, or a salt thereof,

62. The method of claim 49, further defined as a method of activating a subject's B cells comprising contacting the B cells with an effective amount of the oligonucleotide.

63. The method of claim 49, further defined as a method of activating a subject's natural killer cells.

64. The method of claim 49, further defined as a method of treating, preventing or ameliorating an immune system deficiency.

65. The method of claim 49, further defined as a method for ex vivo production of activated lymphocytes.

66. The method of claim 49, further defined as a method of treating a disease associated with an immune system activation.

67. The method of claim 49, further defined as a method of treating systemic lupus erythematosus.

68. The method of claim 49, further defined as a method of treating sepsis.

69. The method of claim 49, further defined as a method of treating or preventing a viral infection.

70. The method of claim 49, further defined as a method of treating a subject having or at risk of having an acute decrement in air flow.

71. The method of claim 49, further defined as a method of inducing an immune response.

72. The method of claim 49, further defined as a method of treating a subject having or at risk of having a viral-mediated disorder.

73. The method of claim 49, further defined as a method of treating a subject having or at risk of having a chronic viral infection.

74. The method of claim 49, wherein said pharmaceutical composition further comprises a nucleic acid encoding an antigenic protein.

75. The method of claim 49, further defined as a method of inducing an immune response.

76. The method of claim 49, further defined as a method of treating a subject having an infectious disorder that is chronic or likely to become chronic.

77. The method of claim 49, further defined as a method of stimulating an immune response in the subject, comprising administering to the subject exposed to an antigen an effective amount for inducing a synergistic antigen specific immune response of an immunopotentiating cytokine and said ODN.

78. The method of claim 77, wherein said ODN comprises at least the following formula:
5′X1, C(dI/dU)X2 3′
wherein the oligonucleotide includes at least 8 nucleotides and wherein X1 and X2 are nucleotides.

79. The method of claim 49, further defined as a method of synergistically activating a dendritic cell, wherein said pharmaceutical composition further comprises a cytokine further defined as GM-CSF, IL-4, TNFa, Flt3 ligand, or IL-3.

80. The method of claim 49, further defined as a method of treating a subject having a neoplastic disorder.

81. The method of claim 80, wherein said pharmaceutical composition further comprises an immunopotentiating cytokine.

82. The method of claim 49, further defined as a method for contraception.

83. The method of claim 82, wherein said pharmaceutical composition further comprises an antigen.

84. The method of claim 83, wherein the antigen is further defined as a gonadal cell antigen or an antigen from a cytokine or hormone required for the maintenance of a gonadal cell.

85. The method of claim 49, further defined as a method of preventing a parasitic infection.

86. The method of claim 49, further defined as a method of treating a subject infected with an eukaryotic parasite.

87. The method of claim 49, further defined as a method of activating the subject's antigen presenting cells.

88. The method of claim 49, wherein said pharmaceutical composition comprises at least one ODN, at least one therapeutic agent, and a therapeutically acceptable carrier.

89. The method of claim 49, wherein said pharmaceutical composition is further defined as a sustained release device.

90. The method of claim 49, wherein said pharmaceutical composition further comprises a polycationic polymer.

91. The method of claim 90, wherein the polycationic polymer is a polycationic peptide.

92. The method of claim 91, wherein the polycationic peptide is a polyarginine, polylysine, an antimicrobial peptide, or a growth hormone.

93. The method of claim 92, wherein the polycationic peptide is a cathelicidin- derived antimicrobial peptide.

94. The method of claim 92, wherein the polycationic peptide is a human growth hormone.

95. The method of claim 49, wherein said pharmaceutical composition further comprises cytokines, anti-inflammatory substances, antimicrobial substances, or combinations thereof.

96. The method of claim 49, wherein said pharmaceutical composition further comprises auxiliary substances, especially a pharmaceutically acceptable carrier, buffer substances, stabilizers or combinations thereof.

97. The method of claim 49, wherein said pharmaceutical composition contains 1 ng to 1 g ODN.

98. The method of claim 97, wherein the pharmaceutical composition contains 100 ng to 10 mg ODN.

99. The method of claim 97, wherein the pharmaceutical composition contains 10 μg to 1 mg ODN.

100. The method of claim 49, further defined as a method of specifically inducing human PBMCs, human myeloid dendritic cells or human plasmacytoid cells.

101. Akitcomprising:
at least one ODN having a structure of formula (I): 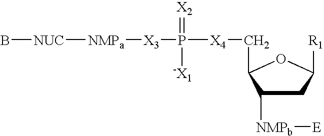
wherein:
R1 is selected from hypoxanthine and uracile,
any X is O or S,
any NMP is a 2′ deoxynucleoside monophosphate or monothiophosphate, further defined as deoxyadenosine-, deoxyguanosine-, deoxyinosine-, deoxycytosine-, deoxyuridine-, deoxythymidine-, 2-methyl-deoxyinosine-, 5-methyl-deoxycytosine-, deoxypseudouridine-, deoxyribosepurine-, 2-amino-deoxyribosepurine-, 6-S-deoxyguanine-, 2-dimethyl-deoxyguanosine- or N-isopentenyl-deoxyadenosine-monophosphate or -monothiophosphate;
NUC is a 2′ deoxynucleoside further defined as deoxyadenosine-, deoxyguanosine-, deoxyinosine-, deoxycytosine-, deoxyinosine-, deoxythymidine-, 2-methyl-deoxyuridine-, 5-methyl-deoxycytosine-, deoxypseudouridine-, deoxyribosepurine-, 2-amino-deoxyribosepurine-, 6-S-deoxyguanine-, 2-dimethyl-deoxyguanosine- or N-isopentenyl-deoxyadenosine;
a and b are integers from 0 to 100 with the proviso that a+b is between 4 and 150;
B and E are common groups for 5′ or 3′ ends of nucleic acid molecules; and at least one therapeutic agent;
wherein said ODN is provided in at least one container and the therapeutic agent is in a separate container.

102. A sustained release device comprising, in a polymer capable of release for at least 7 days:
an ODN having a structure of formula (I): 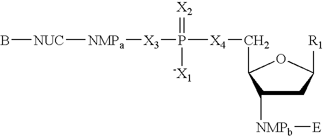
wherein:
R1 is selected from hypoxanthine and uracile,
any X is O or S,
any NMP is a 2′ deoxynucleoside monophosphate or monothiophosphate, further defined as deoxyadenosine-, deoxyguanosine-, deoxyinosine-, deoxycytosine-, deoxyuridine-, deoxythymidine-, 2-methyl-deoxyinosine-, 5-methyl-deoxycytosine-, deoxypseudouridine-, deoxyribosepurine-, 2-amino-deoxyribosepurine-, 6-S-deoxyguanine-, 2-dimethyl-deoxyguanosine- or N-isopentenyl-deoxyadenosine-monophosphate or -monothiophosphate;
NUC is a 2′ deoxynucleoside further defined as deoxyadenosine-, deoxyguanosine-, deoxyinosine-, deoxycytosine-, deoxyinosine-, deoxythymidine-, 2-methyl-deoxyuridine-, 5-methyl-deoxycytosine-, deoxypseudouridine-, deoxyribosepurine-, 2-amino-deoxyribosepurine-, 6-S-deoxyguanine-, 2-dimethyl-deoxyguanosine- or N-isopentenyl-deoxyadenosine;
a and b are integers from 0 to 100 with the proviso that a+b is between 4 and 150;
B and E are common groups for 5′ or 3′ ends of nucleic acid molecules.

103. A pharmaceutical composition comprising an ODN having a structure of formula (I): 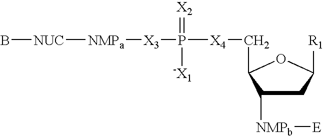
wherein:
R1 is selected from hypoxanthine and uracile,
any X is O or S,
any NMP is a 2′ deoxynucleoside monophosphate or monothiophosphate, further defined as deoxyadenosine-, deoxyguanosine-, deoxyinosine-, deoxycytosine-, deoxyuridine-, deoxythymidine-, 2-methyl-deoxyinosine-, 5-methyl-deoxycytosine-, deoxypseudouridine-, deoxyribosepurine-, 2-amino-deoxyribosepurine-, 6-S-deoxyguanine-, 2-dimethyl-deoxyguanosine- or N-isopentenyl-deoxyadenosine-monophosphate or -monothiophosphate;
NUC is a 2′ deoxynucleoside further defined as deoxyadenosine-, deoxyguanosine-, deoxyinosine-, deoxycytosine-, deoxyinosine-, deoxythymidine-, 2-methyl-deoxyuridine-, 5-methyl-deoxycytosine-, deoxypseudouridine-, deoxyribosepurine-, 2-amino-deoxyribosepurine-, 6-S-deoxyguanine-, 2-dimethyl-deoxyguanosine- or N-isopentenyl-deoxyadenosine;
a and b are integers from 0 to 100 with the proviso that a+b is between 4 and 150; and
B and E are common groups for 5′ or 3′ ends of nucleic acid molecules.


SEQUENCE LISTING (113 sequences)

SEQ ID NO: 1 - DNA, 23 bp, Artificial Sequence
  Description of Artificial Sequence  Synthetic Primer
tccatgacnttcctgctgatgct

SEQ ID NO: 2 - DNA, 20 bp, Artificial Sequence
  Description of Artificial Sequence  Synthetic Primer
nhhhhhwdndhhhhhhhhwn

SEQ ID NO: 3 - DNA, 10 bp, Artificial Sequence
  Description of Artificial Sequence  Synthetic Primer
hhhwdndhhh

SEQ ID NO: 4 - DNA, 10 bp, Artificial Sequence
  Description of Artificial Sequence  Synthetic Primer
hhhwdndhhh

SEQ ID NO: 5 - DNA, 20 bp, Artificial Sequence
  Description of Artificial Sequence  Synthetic Primer
nhhhhhwdnnhhhhhhhhwn

SEQ ID NO: 6 - DNA, 20 bp, Artificial Sequence
  Description of Artificial Sequence  Synthetic Primer
nhhwdndnnhhhhdnndnnh

SEQ ID NO: 7 - DNA, 20 bp, Artificial Sequence
  Description of Artificial Sequence  Synthetic Primer
nhhhhhwdndhhhhhhhhwn

SEQ ID NO: 8 - DNA, 20 bp, Artificial Sequence
  Description of Artificial Sequence  Synthetic Primer
nhhwdndndhhhhdnddndh

SEQ ID NO: 9 - protein, 8 aa, Mus musculus
VYDFFVWL

SEQ ID NO: 10 - protein, 8 aa, Gallus gallus
SIINFEKL

SEQ ID NO: 11 - protein, 9 aa, Mus musculus
LPYLGWLVF

SEQ ID NO: 12 - DNA, 20 bp, Artificial Sequence
  Description of Artificial Sequence  Synthetic Primer
tccatgacgttcctgatgct

SEQ ID NO: 13 - DNA, 20 bp, Artificial Sequence
  Description of Artificial Sequence  Synthetic Primer
tccatnacnttcctgatgct

SEQ ID NO: 14 - DNA, 20 bp, Artificial Sequence
  Description of Artificial Sequence  Synthetic Primer
tccatgacnttcctgatgct

SEQ ID NO: 15 - DNA, 20 bp, Artificial Sequence
  Description of Artificial Sequence  Synthetic Primer
tccatnacnttcctnatnct

SEQ ID NO: 16 - DNA, 15 bp, Artificial Sequence
  Description of Artificial Sequence  Synthetic Primer
tccatgagcttcctg

SEQ ID NO: 17 - DNA, 20 bp, Artificial Sequence
  Description of Artificial Sequence  Synthetic Primer
tccatgancttcctgatgct

SEQ ID NO: 18 - DNA, 20 bp, Artificial Sequence
  Description of Artificial Sequence  Synthetic Primer
tccatnancttcctnatnct

SEQ ID NO: 19 - DNA, 10 bp, Artificial Sequence
  Description of Artificial Sequence  Synthetic Primer
hhhwdndhhh

SEQ ID NO: 20 - protein, 9 aa, Artificial Sequence
  Description of Artificial Sequence  Synthetic Peptide
SYVPSAEQI

SEQ ID NO: 21 - DNA, 10 bp, Artificial Sequence
  Description of Artificial Sequence  Synthetic Primer
hhhwdddhhh

SEQ ID NO: 22 - DNA, 24 bp, Artificial Sequence
  Description of Artificial Sequence  Synthetic Primer
tcgtcgttttgtcgttttgtcgtt

SEQ ID NO: 23 - DNA, 24 bp, Artificial Sequence
  Description of Artificial Sequence  Synthetic Primer
ncncncncncncncncncncncnc

SEQ ID NO: 24 - protein, 11 aa, Artificial Sequence
  Description of Artificial Sequence  Synthetic Peptide
KLKLLLLLKLK

SEQ ID NO: 25 - DNA, 20 bp, Artificial Sequence
  Description of Artificial Sequence  Synthetic Primer
tcntcnttgtcnttgtcntt

SEQ ID NO: 26 - DNA, 24 bp, Artificial Sequence
  Description of Artificial Sequence  Synthetic Primer
tcntcnttttgtcnttttgtcntt

SEQ ID NO: 27 - DNA, 22 bp, Artificial Sequence
  Description of Artificial Sequence  Synthetic Primer
tcntcnttgtcnttttgtcntt

SEQ ID NO: 28 - DNA, 21 bp, Artificial Sequence
  Description of Artificial Sequence  Synthetic Primer
gcntgcnttgtcnttgtcntt

SEQ ID NO: 29 - DNA, 21 bp, Artificial Sequence
  Description of Artificial Sequence  Synthetic Primer
tgtcntttgtcntttgtcntt

SEQ ID NO: 30 - DNA, 19 bp, Artificial Sequence
  Description of Artificial Sequence  Synthetic Primer
tgtcnttgtcnttgtcntt

SEQ ID NO: 31 - DNA, 14 bp, Artificial Sequence
  Description of Artificial Sequence  Synthetic Primer
tcntcntcntcntt

SEQ ID NO: 32 - DNA, 20 bp, Artificial Sequence
  Description of Artificial Sequence  Synthetic Primer
tcctgtcnttccttgtcntt

SEQ ID NO: 33 - DNA, 20 bp, Artificial Sequence
  Description of Artificial Sequence  Synthetic Primer
tcctgtcnttttttgtcntt

SEQ ID NO: 34 - DNA, 21 bp, Artificial Sequence
  Description of Artificial Sequence  Synthetic Primer
tcntcnctgtctgcccttctt

SEQ ID NO: 35 - DNA, 21 bp, Artificial Sequence
  Description of Artificial Sequence  Synthetic Primer
tcntcnctgttgtcntttctt

SEQ ID NO: 36 - DNA, 19 bp, Artificial Sequence
  Description of Artificial Sequence  Synthetic Primer
tgtcnttgtcnttgtcntt

SEQ ID NO: 37 - DNA, 20 bp, Artificial Sequence
  Description of Artificial Sequence  Synthetic Primer
tccatgacnttcctgacntt

SEQ ID NO: 38 - DNA, 20 bp, Artificial Sequence
  Description of Artificial Sequence  Synthetic Primer
tccatgacnttcctgacntt

SEQ ID NO: 39 - DNA, 24 bp, Artificial Sequence
  Description of Artificial Sequence  Synthetic Primer
accatggacnatctgtttcccctc

SEQ ID NO: 40 - DNA, 18 bp, Artificial Sequence
  Description of Artificial Sequence  Synthetic Primer
tctcccagcntgcnccat

SEQ ID NO: 41 - DNA, 18 bp, Artificial Sequence
  Description of Artificial Sequence  Synthetic Primer
taccncntgcnaccctct

SEQ ID NO: 42 - DNA, 24 bp, Artificial Sequence
  Description of Artificial Sequence  Synthetic Primer
accatggacnaactgtttcccctc

SEQ ID NO: 43 - DNA, 24 bp, Artificial Sequence
  Description of Artificial Sequence  Synthetic Primer
accatggacnagctgtttcccctc

SEQ ID NO: 44 - DNA, 24 bp, Artificial Sequence
  Description of Artificial Sequence  Synthetic Primer
accatggacnacctgtttcccctc

SEQ ID NO: 45 - DNA, 24 bp, Artificial Sequence
  Description of Artificial Sequence  Synthetic Primer
accatggacntactgtttcccctc

SEQ ID NO: 46 - DNA, 24 bp, Artificial Sequence
  Description of Artificial Sequence  Synthetic Primer
accatggacngtctgtttcccctc

SEQ ID NO: 47 - DNA, 24 bp, Artificial Sequence
  Description of Artificial Sequence  Synthetic Primer
accatggacnttctgtttcccctc

SEQ ID NO: 48 - DNA, 15 bp, Artificial Sequence
  Description of Artificial Sequence  Synthetic Primer
gcatgacnttgagct

SEQ ID NO: 49 - DNA, 15 bp, Artificial Sequence
  Description of Artificial Sequence  Synthetic Primer
cacnttgaggggcat

SEQ ID NO: 50 - DNA, 15 bp, Artificial Sequence
  Description of Artificial Sequence  Synthetic Primer
ctgctgagactggag

SEQ ID NO: 51 - DNA, 12 bp, Artificial Sequence
  Description of Artificial Sequence  Synthetic Primer
tcagcntgcncc

SEQ ID NO: 52 - DNA, 17 bp, Artificial Sequence
  Description of Artificial Sequence  Synthetic Primer
atgacnttcctgacntt

SEQ ID NO: 53 - DNA, 17 bp, Artificial Sequence
  Description of Artificial Sequence  Synthetic Primer
tctcccagcnggcncat

SEQ ID NO: 54 - DNA, 18 bp, Artificial Sequence
  Description of Artificial Sequence  Synthetic Primer
tctcccagcncncnccat

SEQ ID NO: 55 - DNA, 20 bp, Artificial Sequence
  Description of Artificial Sequence  Synthetic Primer
tccatgtcnttcctgtcntt

SEQ ID NO: 56 - DNA, 20 bp, Artificial Sequence
  Description of Artificial Sequence  Synthetic Primer
tccatagcnttcctagcntt

SEQ ID NO: 57 - DNA, 21 bp, Artificial Sequence
  Description of Artificial Sequence  Synthetic Primer
tcntcnctgtctccncttctt

SEQ ID NO: 58 - DNA, 19 bp, Artificial Sequence
  Description of Artificial Sequence  Synthetic Primer
tcctgacnttcctgacntt

SEQ ID NO: 59 - DNA, 20 bp, Artificial Sequence
  Description of Artificial Sequence  Synthetic Primer
tccatgtngttcctgtngtt

SEQ ID NO: 60 - DNA, 20 bp, Artificial Sequence
  Description of Artificial Sequence  Synthetic Primer
tccaggacttctctcaggtt

SEQ ID NO: 61 - DNA, 20 bp, Artificial Sequence
  Description of Artificial Sequence  Synthetic Primer
tccatgcntgcntgcntttt

SEQ ID NO: 62 - DNA, 20 bp, Artificial Sequence
  Description of Artificial Sequence  Synthetic Primer
tccatgcnttgcnttgcntt

SEQ ID NO: 63 - DNA, 20 bp, Artificial Sequence
  Description of Artificial Sequence  Synthetic Primer
tccacnacnttttcnacntt

SEQ ID NO: 64 - DNA, 23 bp, Artificial Sequence
  Description of Artificial Sequence  Synthetic Primer
ntcntcnttgtcnttttgtcntt

SEQ ID NO: 65 - DNA, 20 bp, Artificial Sequence
  Description of Artificial Sequence  Synthetic Primer
gcngcnggcngcncncnccc

SEQ ID NO: 66 - DNA, 25 bp, Artificial Sequence
  Description of Artificial Sequence  Synthetic Primer
tgtcnttgtcnttgtcnttgtcntt

SEQ ID NO: 67 - DNA, 13 bp, Artificial Sequence
  Description of Artificial Sequence  Synthetic Primer
tgtcnttgtcntt

SEQ ID NO: 68 - DNA, 15 bp, Artificial Sequence
  Description of Artificial Sequence  Synthetic Primer
tccaacnttntcntt

SEQ ID NO: 69 - DNA, 19 bp, Artificial Sequence
  Description of Artificial Sequence  Synthetic Primer
gcntgcnttgtcnttgttt

SEQ ID NO: 70 - DNA, 19 bp, Artificial Sequence
  Description of Artificial Sequence  Synthetic Primer
tgtcnttgtttgtttgttt

SEQ ID NO: 71 - DNA, 20 bp, Artificial Sequence
  Description of Artificial Sequence  Synthetic Primer
tccatgacnttcctgatgct

SEQ ID NO: 72 - DNA, 20 bp, Artificial Sequence
  Description of Artificial Sequence  Synthetic Primer
tccatgtcnctcctgatgct

SEQ ID NO: 73 - DNA, 20 bp, Artificial Sequence
  Description of Artificial Sequence  Synthetic Primer
tccatgtcnttcctgatgct

SEQ ID NO: 74 - DNA, 15 bp, Artificial Sequence
  Description of Artificial Sequence  Synthetic Primer
gctagacnttagcnt

SEQ ID NO: 75 - DNA, 15 bp, Artificial Sequence
  Description of Artificial Sequence  Synthetic Primer
gctagatgttagcnt

SEQ ID NO: 76 - DNA, 15 bp, Artificial Sequence
  Description of Artificial Sequence  Synthetic Primer
gctagacnttagngt

SEQ ID NO: 77 - DNA, 20 bp, Artificial Sequence
  Description of Artificial Sequence  Synthetic Primer
atggaaggtccagcnttctc

SEQ ID NO: 78 - DNA, 20 bp, Artificial Sequence
  Description of Artificial Sequence  Synthetic Primer
atcnactctcnagcnttctc

SEQ ID NO: 79 - DNA, 20 bp, Artificial Sequence
  Description of Artificial Sequence  Synthetic Primer
atngactctcnagcnttctc

SEQ ID NO: 80 - DNA, 20 bp, Artificial Sequence
  Description of Artificial Sequence  Synthetic Primer
atcnactctcnagcnttntc

SEQ ID NO: 81 - DNA, 20 bp, Artificial Sequence
  Description of Artificial Sequence  Synthetic Primer
atcnactctcnaacnttctc

SEQ ID NO: 82 - DNA, 20 bp, Artificial Sequence
  Description of Artificial Sequence  Synthetic Primer
gagaacnctggaccttccat

SEQ ID NO: 83 - DNA, 20 bp, Artificial Sequence
  Description of Artificial Sequence  Synthetic Primer
gagaacnctcnaccttccat

SEQ ID NO: 84 - DNA, 20 bp, Artificial Sequence
  Description of Artificial Sequence  Synthetic Primer
gagaacnctcnaccttcnat

SEQ ID NO: 85 - DNA, 20 bp, Artificial Sequence
  Description of Artificial Sequence  Synthetic Primer
gagcaagctggaccttccat

SEQ ID NO: 86 - DNA, 20 bp, Artificial Sequence
  Description of Artificial Sequence  Synthetic Primer
gagaacnctggacnttccat

SEQ ID NO: 87 - DNA, 20 bp, Artificial Sequence
  Description of Artificial Sequence  Synthetic Primer
gagaacnatggaccttccat

SEQ ID NO: 88 - DNA, 20 bp, Artificial Sequence
  Description of Artificial Sequence  Synthetic Primer
gagaacnctccagcactgat

SEQ ID NO: 89 - DNA, 19 bp, Artificial Sequence
  Description of Artificial Sequence  Synthetic Primer
ccatgtcngtcctgatgct

SEQ ID NO: 90 - DNA, 20 bp, Artificial Sequence
  Description of Artificial Sequence  Synthetic Primer
tccatgtcngtnctgatgct

SEQ ID NO: 91 - DNA, 20 bp, Artificial Sequence
  Description of Artificial Sequence  Synthetic Primer
tccatgtcngtcctgacnca

SEQ ID NO: 92 - DNA, 20 bp, Artificial Sequence
  Description of Artificial Sequence  Synthetic Primer
atggactctccagcnttctc

SEQ ID NO: 93 - DNA, 20 bp, Artificial Sequence
  Description of Artificial Sequence  Synthetic Primer
ataggaggtccaacnttctc

SEQ ID NO: 94 - DNA, 20 bp, Artificial Sequence
  Description of Artificial Sequence  Synthetic Primer
atggaggctccatcnttctc

SEQ ID NO: 95 - DNA, 20 bp, Artificial Sequence
  Description of Artificial Sequence  Synthetic Primer
atcnactctcnagngttctc

SEQ ID NO: 96 - DNA, 20 bp, Artificial Sequence
  Description of Artificial Sequence  Synthetic Primer
tccatgtcngtcctgatgct

SEQ ID NO: 97 - DNA, 20 bp, Artificial Sequence
  Description of Artificial Sequence  Synthetic Primer
tccatggcngtcctgatgct

SEQ ID NO: 98 - DNA, 20 bp, Artificial Sequence
  Description of Artificial Sequence  Synthetic Primer
tccatgacngtcctgatgct

SEQ ID NO: 99 - DNA, 20 bp, Artificial Sequence
  Description of Artificial Sequence  Synthetic Primer
tccatgtcnatcctgatgct

SEQ ID NO: 100 - DNA, 20 bp, Artificial Sequence
  Description of Artificial Sequence  Synthetic Primer
tccataacnttcctgatgct

SEQ ID NO: 101 - DNA, 20 bp, Artificial Sequence
  Description of Artificial Sequence  Synthetic Primer
tccatgacntccctgatgct

SEQ ID NO: 102 - DNA, 20 bp, Artificial Sequence
  Description of Artificial Sequence  Synthetic Primer
tccatcacntgcctgatgct

SEQ ID NO: 103 - DNA, 19 bp, Artificial Sequence
  Description of Artificial Sequence  Synthetic Primer
ggggtcaacnttgacnggg

SEQ ID NO: 104 - DNA, 19 bp, Artificial Sequence
  Description of Artificial Sequence  Synthetic Primer
ggggtcagtcntgacnggg

SEQ ID NO: 105 - DNA, 15 bp, Artificial Sequence
  Description of Artificial Sequence  Synthetic Primer
gctagacnttagtgt

SEQ ID NO: 106 - DNA, 20 bp, Artificial Sequence
  Description of Artificial Sequence  Synthetic Primer
tccatgtcntttttgtcntt

SEQ ID NO: 107 - DNA, 20 bp, Artificial Sequence
  Description of Artificial Sequence  Synthetic Primer
tccttgtcnttcctgtcntt

SEQ ID NO: 108 - DNA, 20 bp, Artificial Sequence
  Description of Artificial Sequence  Synthetic Primer
tccatgtcntttttgtcntt

SEQ ID NO: 109 - DNA, 20 bp, Artificial Sequence
  Description of Artificial Sequence  Synthetic Primer
tccttgtcnttcctgtcntt

SEQ ID NO: 110 - DNA, 19 bp, Artificial Sequence
  Description of Artificial Sequence  Synthetic Primer
tccaacntttcacacactt

SEQ ID NO: 111 - DNA, 19 bp, Artificial Sequence
  Description of Artificial Sequence  Synthetic Primer
tcctgtcnttcctgtcntt

SEQ ID NO: 112 - DNA, 20 bp, Artificial Sequence
  Description of Artificial Sequence  Synthetic Primer
tccatgtcntttttgtcntt

SEQ ID NO: 113 - DNA, 20 bp, Artificial Sequence
  Description of Artificial Sequence  Synthetic Primer
tccttgtcnttcctgtcntt